(19) United States
(12) Patent Application Publication
(10) Pub. No.: US 2025/0027073 A1
(43) Pub. Date: Jan. 23, 2025

(54) METHODS AND COMPOSITIONS FOR DISCOVERY OF RECEPTOR-LIGAND SPECIFICITY BY ENGINEERED CELL ENTRY

(71) Applicant: The Board of Trustees of the Leland Stanford Junior University, Stanford, CA (US)

(72) Inventors: Howard Y. CHANG, Stanford, CA (US); Ansuman SATPATHY, Stanford, CA (US); Bingfei YU, Stanford, CA (US); Quanming SHI, Stanford, CA (US)

(21) Appl. No.: 18/716,869

(22) PCT Filed: Dec. 5, 2022
(86) PCT No.: PCT/US2022/080915

§ 371 Date: Jun. 5, 2024

Related U.S. Application Data
(60) Provisional application No. 63/382,860, filed on Nov. 8, 2022.
(60) Provisional application No. 63/286,507, filed on Dec. 6, 2021.

(51) Int. Cl. C12N 15/10

(52) U.S. Cl. CPC C12N 15/1037; C12N 15/1065; C12N 15/1082; C12N 15/1086

(57) ABSTRACT

The present disclosure relates to systems, methods and compositions for decoding ligand-receptor interactions, for delivering nucleic acids and proteins into target cells and for performing single cell multiomics. Disclosed are engineered lentiviruses displaying ligands that deliver cargo into target cells upon cognate receptor-ligand interaction. Also disclosed are compositions and methods including pMHC or antigen epitopes displaying lentiviruses for identifying pMHC/T-cell receptors and antigen/B-cell receptor interactions.

### CROSS-REFERENCE TO RELATED APPLICATIONS

This application claims the benefit of priority to U.S. Provisional patent application Ser. No. 63/286,507, filed on Dec. 6, 2021, and U.S. Provisional patent application Ser. No. 63/382,860, filed on Nov. 8, 2022. The disclosures of the above-referenced applications are herein expressly incorporated by reference it their entireties, including any drawings.

### INCORPORATION OF THE SEQUENCE LISTING

The material in the accompanying Sequence Listing is hereby incorporated by reference into this application. The accompanying Sequence Listing text file, named 078430-538001WO_Sequence Listing_ST26.xml, was created on Dec. 1, 2022, and is 17 KB.

### FIELD

The technology relates generally to the field of cell biology. More particularly, the technology relates to methods and compositions for the discovery and identification of ligand-receptor specificity, and for gene and protein delivery. The technology also relates to decoding interactions between T-cell receptors and MHC peptides, antibodies and antigens, or B-cell receptors and B cell antigens, including intracellular/secreted epitopes/cell-surface antigen epitopes, as well as other ligand-receptors.

### BACKGROUND

Cells communicate with each other through ligand-receptor interactions. Rich intercellular communications shape the molecular programs of mammalian cells to instruct specific behaviors and cell fate decisions. For example, TCR on surface of T cells can recognize and interact with the major histocompatibility complex (MHC)-antigen complexes from the surface of antigen-presenting cells (APC). TCR and antibody genes undergo somatic recombination to reach a large and diverse repertoire (˜10¹⁶ TCR alpha and beta sequences in humans), which are clonally inherited by daughter cells. T and B cell receptor interactions are highly specific and drive antigen-specific T and B cell expansion and differentiation. Resolving TCR-antigen interactions, especially linking antigen specificity to TCR sequences and T cell states are essential to understanding how antigen recognition drives T cell fate decisions. Diverse approaches have been developed to decipher the antigen specificity of TCRs including: (1) Cell reporter assay to screen T cell-specific MHC-antigens using artificial APCs such as T-scan, SABR, T cell trogocytosis, and cytokine capturing assay (Joglekar et al., 2019; Kula et al., 2019; Lee and Meyerson, 2021; Li et al., 2019); (2) Yeast display platform to screen MHC-antigens for recombinant TCRs (Birnbaum et al., 2012); (3) T cell based assay such as cytokine production (ELISpot) upon antigen peptide stimulation (McCutcheon et al., 1997); (4) DNA barcoded MHC-peptide multimer to capture antigen specificity and TCR sequence by single cell sequencing (TetTCR-seq; Zhang et al., 2018). Despite their unique advantages for each technique, it is still challenging to rapidly screen immunogenic MHC-antigens for primary T cells and simultaneously capture the antigen landscape, paired TCR repertoire, and gene expression of T cell phenotypes in a high-throughput manner. Many existing methods require re-expression of receptors or ligands on heterologous cells, and thus cannot be applied directly to human clinical samples. Similar challenges apply to study B cell receptor-antigen interactions, with the added challenge of addressing known intracellular antigen epitopes recognized by antibodies.

Despite extensive progress in characterizing the cell state of antigen-specific T and B cells, it remains challenging to target these antigen-specific cells and selectively rewire their cell state and behavior without perturbing other bystander T or B cells. A recent method using pMHC presenting nanoparticles has enabled mRNA delivery in antigen-specific T cells, opening many possibilities to transiently modulate specific T cells (Su et al., 2022). Another recent study using pMHC pseudotyped viruses allows genetic modification of antigen-specific T cells (Guo and Elledge, 2022). However, there is still a lack of technology to selectively manipulate antigen-specific B cells beyond antigen-specific T cells.

Accordingly, there is a need for methods and compositions for a reliable and rapid systematic identification of ligand-receptor pairing and for decoding receptor specificity. More particularly, there is a need for a method that can scalably (1) display many different types of ligands, (2) match the ligand to receptors on cells, (3) record the information, (4) manipulate the cells that express receptors that match the ligand. There is also a need for methods and compositions for exploring ligand-receptor pairing at a single-cell resolution, and for cell-specific delivery of gene or protein payloads.

### SUMMARY

In one aspect, the present disclosure provides a engineered lentivirus comprising a heterologous ligand displayed on a surface of the lentivirus, a fusogen comprising a modified viral envelope protein, wherein the fusogen is capable of fusing the lentivirus to a host cell, wherein the host cell comprises an endogenous receptor for the ligand; a reporter protein operably linked, e.g., fused to a lentiviral structural protein; and a barcoded RNA.

Non-limiting exemplary embodiments of the engineered lentiviruses of the disclosure can include one or more of the following features. In some embodiments, the fusogen comprises a modified VSV-G viral envelope protein. In some embodiments, wherein the modified VSV-G viral envelope protein comprises one or more amino acid substitutions at any one of the positions H8, K47, Y209, and R354 of the VSV-G polypeptide. In some embodiments, the modified VSV-G viral envelope protein comprises a K47Q substitution and a R354A substitution.

In some embodiments, the ligand is or comprises a protein or an epitope.

In some embodiments, the ligand is or comprises a MHC peptide, an antibody, an antigen, a secreted protein, a cell-surface protein, or other form of antigen that may be expressed by a cell. In some embodiments, the antigen is or comprises an intracellular antigen.

In some embodiments, the ligand is operably linked, e.g., fused to an optimized transmembrane domain. In some embodiments, the optimized transmembrane domain is transmembrane domain derived from HLA-DRA, HLA-DRB, HLA-A2, ICAM1, CD43, CD162, CD62L, CD49d, or LFA-1.

In some embodiments, the ligand is operably linked, e.g., fused to an optimized transmembrane domain and a signal peptide.

In some embodiments, the engineered lentivirus comprises a defective integrase protein.

In some embodiments, the reporter protein is GFP or mNeon.

In some embodiments, the structural protein is a nucleocapsid protein.

In some embodiments, the structural protein is a Gag protein.

In some embodiments, the barcoded RNA is encapsulated in viral particles.

In some embodiments, the RNA encodes the ligand, e.g., protein ligand.

In some embodiments, the RNA encodes a gene of interest to be delivered into target cells, e.g., host cells.

In some embodiments, the RNA is read out by a next-generation sequencing technology.

In some embodiments, the RNA comprises a capture sequence, e.g., a sequence that can be used to capture or hybridize to an analyte (e.g., DNA, RNA, protein) from or within a sample, e.g., in a 10× Genomics single-cell sequencing workflow.

In one aspect, the present disclosure further provides a method for identifying a ligand-receptor pair, the method comprising providing at least one engineered lentivirus comprising a heterologous ligand displayed on a surface of the lentivirus, a fusogen comprising a modified viral envelope protein, wherein the fusogen is capable of fusing the lentivirus to a host cell, wherein the host cell comprises an endogenous receptor, e.g., immune receptor, for the ligand; a reporter protein fused to a lentiviral structural protein; and a barcoded RNA, combining the lentivirus with a population of cells, and sorting the population of cells based on the presence of the reporter gene, thereby identifying a ligand-receptor pair.

Non-limiting exemplary embodiments of the methods for identifying a ligand-receptor pair of the disclosure can include one or more of the following features. In some embodiments, the method comprises providing a pool of engineered lentiviruses, the pool displaying different ligands.

In some embodiments, the method comprises combining the lentivirus with cells and incubating the virus/cell mixture at about 4° C.

In some embodiments, the method comprises combining the lentivirus with cells, and incubating the virus/cell mixture at room temperature.

In some embodiments, the method comprises combining the lentivirus with cells and incubating the virus/cell mixture at about 37° C.

In some embodiments, the method comprises incubating the virus/cell mixture for about 30 minutes, or about 1 hour or about 2 hours or any period from about 0.5 hours to about 2.5 hours.

In some embodiments, the method further comprises the step of single cell sequencing of the viral RNA to identify the ligand sequence.

In some embodiments, the method further comprises the step of single cell sequencing of the cell's transcriptome to identify receptor sequence.

In one aspect, the present disclosure further provides a method of delivering a molecule of interest, e.g., a nucleic acid or a protein of interest, to a user-defined target cell, comprising providing an engineered lentivirus comprising a heterologous ligand displayed on a surface of the lentivirus, a fusogen comprising a modified viral envelope protein, wherein the fusogen is capable of fusing the lentivirus to a host cell, wherein the host cell comprises an endogenous receptor, e.g., immune receptor, for the ligand; a reporter protein fused to a lentiviral structural protein; and a barcoded RNA, contacting the lentivirus with a cell mixture comprising the target cell, and delivering the nucleic acid or protein only to the target cell, wherein the target cell expresses a receptor, e.g., immune receptor, specific to the ligand on the lentivirus surface.

Non-limiting exemplary embodiments of the methods for delivering a molecule of interest of the disclosure can include one or more of the following features. In some embodiments, the ligand is modified in order to deliver cargo to the user-defined target cell.

In some embodiments, the nucleic acid of interest is packaged inside the engineered lentiviral particle.

In some embodiments, the protein of interest is operably linked to, e.g., fused with a gag protein of the lentivirus.

In some embodiments, the protein of interest replaces the reporter. In some embodiments, the target cell is in vivo. In some embodiments, the target cell is ex vivo. In some embodiments, the target cell is in vitro. In some embodiments, the target cell is a mammalian cell. In some embodiments, the mammalian cell is a human cell. In some embodiments, the target cell is an immune cell. In some embodiments, the immune cell is a T cell or a B cell. In some embodiments, the immune cell is a T cell and the receptor is a T-cell receptor. In some embodiments, the immune cell is a B cell and the receptor is a B-cell receptor. In some embodiments, the human cell is a primary human blood cell (PBMC).

In another aspect, the present disclosure further provides a method for identifying an immunogenic antigen, the method comprising providing an engineered lentivirus comprising a heterologous receptor protein displayed on the surface of the lentivirus, a fusogen comprising a modified VSV-G viral envelope protein wherein the fusogen is capable of fusing the lentivirus to a host cell, wherein the host cell comprises a native antigen for the receptor, a reporter transgene fused to a lentivirus structural protein, and a barcoded RNA, wherein the RNA encodes antigen information, combining the lentivirus with the population of cells and sorting the population of cells based on the presence of the reporter.

Non-limiting exemplary embodiments of the methods for identifying an immunogenic antigen of the disclosure can include one or more of the following features. In some embodiments, the method further comprises sequencing the viral RNA to identify the antigen sequence.

In some embodiments, the method further comprises a step for sequencing of the cell's receptor RNA.

In another aspect, the present disclosure further provides a method of identifying a T-cell receptor and paired MHC-peptide, the method comprising providing an engineered lentivirus comprising a pMHC displayed on virus surface, a fusogen comprising a modified VSV-G viral envelope protein wherein the fusogen is capable of fusing the lentivirus to a host cell, wherein the host cell comprises a T-cell receptor for the PMHC, a reporter transgene fused to a lentivirus structural protein, and a barcoded RNA, combining the lentivirus with the population of cells and sorting the population of cells based on the presence of the reporter thereby identifying the T-cell receptor.

Non-limiting exemplary embodiments of the methods for identifying a T-cell receptor and paired MHC-peptide of the disclosure can include one or more of the following features. In some embodiments, the population of cells comprises human primary T-cells.

In some embodiments, the PMHC is encoded by a RNA comprising a signal peptide, the PMHC, a G4S linker, b2m gene, a G4S linker and a MH allele in tandem.

In some embodiments, the method further comprises a step for single cell sequencing of the viral RNA to identify the MHC peptide sequence.

In some embodiments, the method further comprises a step for sequencing of the cell's receptor sequence to identify the MHC peptide and T-cell receptor sequence.

In yet another aspect, the present disclosure also provides a method of identifying a B-cell receptor or antibody, the method comprising providing an engineered lentivirus comprising an epitope displayed on lentivirus surface wherein the epitope is operably linked to, e.g., fused with an ICAM1 transmembrane domain, a fusogen comprising a modified VSV-G viral envelope protein wherein the fusogen is capable of fusing the lentivirus to a host cell, wherein the host cell comprises a B-cell receptor for the intracellular epitope, a reporter transgene fused to a lentivirus structural protein, and a barcoded RNA, combining the lentivirus with the population of B cells and sorting the population of cells based on the presence of the reporter thereby identifying the B-cell receptor or antibody.

Non-limiting exemplary embodiments of the methods for identifying a B-cell receptor or antibody of the disclosure can include one or more of the following features. In some embodiments, the antigen is a cell surface membrane protein, an intracellular protein, a secreted protein, or glycosylated protein.

In some embodiments, the method further comprises the step of single cell sequencing of the viral RNA to identify the antigen and matching B-cell receptor sequences.

The present disclosure further provides at least one embodiment of a method of identifying an antigen for a B-cell receptor, the method comprising providing an engineered lentivirus comprising a heterologous ligand displayed on a surface of the lentivirus, a fusogen comprising a modified viral envelope protein, wherein the fusogen is capable of fusing the lentivirus to a host cell, wherein the host cell comprises an endogenous receptor for the ligand; a reporter protein fused to a lentiviral structural protein; and a barcoded RNA, combining the lentivirus with the population of B cells and sorting the population of cells based on the presence of the reporter thereby identifying the B-cell antigen.

The present disclosure also provides at least one embodiment of a method of single cell multiomics, comprising providing an engineered lentivirus comprising a heterologous ligand displayed on the surface of the lentivirus, a fusogen comprising a modified VSV-G viral envelope protein wherein the fusogen is capable of fusing the lentivirus to a host cell, wherein the host cell comprises a an endogenous receptor for the ligand, a reporter transgene fused to a lentivirus structural protein, and a RNA, wherein the RNA comprises an antigen sequence and a capture tag for single cell sequencing and retrieving transcriptome and phenotype information simultaneously at the single cell level.

In some embodiments, the single cells sequencing is a droplet based platform.

In some embodiments, the cell's phenotype comprise surface markers by CITE-seq.

In some embodiments, the information comprises ligand and receptor sequences.

In some embodiments, the single cells multiomics use whole cell as input.

In some embodiments, the single cells multiomics use whole cell as input and comprises a step of reverse transcription.

In yet another aspect, the present disclosure provide a method for selectively depleting or enriching a target cell population in a cell mixture, the method including: providing (a) an engineered lentivirus as described herein, and (b) a cell mixture comprising (i) a target cell population expressing a receptor specific for the ligand displayed on the surface of the engineered lentivirus, and (ii) a non-target cell population that does not express the receptor specific for the ligand displayed on the surface of the engineered lentivirus; contacting the engineered lentivirus with the cell population, and delivering the nucleic acid or protein only to the target cell; adding a reagent that specifically inhibits growth of the target cell population or inhibits growth of the non-target cell population, thereby selectively depleting or enriching the target cell population. In some embodiments, the receptor is an immune receptor. In some embodiments, the immune receptor is a B-cell receptor. In some embodiments, immune receptor is a T-cell receptor.

Non-limiting exemplary embodiments of the methods for selectively depleting or enriching a target cell population of the disclosure can include one or more of the following features. In some embodiments, the target cell expresses a herpes simplex virus thymidine kinase (HSV-TK) transgene, and the added reagent comprises or is ganciclovir (GCV). In some embodiments, the target cell expresses shRNA to decrease expression of cell death receptor FAS to prevent cell death of the target population. In some embodiments, the target cell population comprises immune cells. In some embodiments, the immune cells comprise a T cell. In some embodiments, the immune cells comprise a B cell.

In some embodiments, the immune cells are autoreactive immune cells. In some embodiments, the immune cells are specific for an antigen associated with a health condition. In some embodiments, the health condition is a proliferative disorder, inflammatory disorder, autoimmune disorder, or a microbial infection. In some embodiments, the proliferative disorder is a cancer. In some embodiments, the microbial infection is a bacterial infection, viral infection, or microfungal infection.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### BRIEF DESCRIPTION OF THE DRAWINGS

FIGS. 1A-1G illustrate a viral display platform to display ligand proteins and fusogen on viral surface, deliver fluorescent proteins, and record ligand-receptor interaction by cell entry. FIG. 1A shows a schematic view of an exemplary all-in-one platform. The lentiviruses are engineered in diverse components including: (1) user-defined ligand proteins displayed on viral surface; (2) modified fusogen with intact fusion ability and defective binding to natural receptors; (3) cargo proteins fused with viral structure protein; and (4) barcoded viral RNA for tracing and gene delivery.

FIG. 1B shows a schematic view of an experimental set up and flow cytometry analysis of GFP expression after 3 days of viral infection. Raji and Jurkat cells are infected by three groups of lentiviruses encoding GFP in the viral RNA: (1) viruses with wild-type VSV-G (left); (2) viruses with receptor-binding mutated VSV-G (middle); (3) viruses with VSV-G mutant and anti-CD19 single-chain antibody variable fragment (scFv).

FIG. 1C shows a schematic view (top) of an experimental set up. GFP protein are fused with matrix protein (MA-GFP) or Nucleocapsid protein (NC-GFP), or viral protein R (VPR-GFP). scFv-CD19 displayed viruses carrying GFP protein fused with different viral proteins were incubated with Raji (CD19+) or Jurkat (CD19−) cells for 2 hours and then subjected to flow cytometry. Bar plot (bottom) showing the percentage of GFP+ cells upon incubation of viruses with different GFP fusion viral proteins.

FIG. 1D shows exemplary flow cytometry plots of GFP signal after transient viral incubation as in FIG. 1C.

FIG. 1E shows schematic view of experimental set up and flow cytometry analysis of GFP signal in primary human B cells with or without viral incubation. Naïve and activated human primary B cells were incubated with NC-GFP fused and scFv-CD19 displayed viruses for 2 hours and then subject to flow cytometry analysis. B cells were gated on live CD20+ cells.

FIG. 1F is a histogram analysis of surface CD19 expression of groups from FIG. 1E.

FIG. 1G depicts bar plots showing scFv-CD19 virus binding and CD19 surface expression in naïve and activated human B cells. P-values in FIGS. 1C and 1F are calculated by unpaired t-test. ****P<0.0001***P<0.001.

FIGS. 1H-1N illustrate how to decipher a specific interaction of ligand-receptor for co-stimulatory molecules by ENTER.

FIG. 1H depicts a Flow cytometry analysis of the viral binding and fusion of scFv-CD19 displayed viruses on Raji B cells at different temperature before and after proteinase K treatment.

FIG. 1I is a Bar plot showing the percentage of GFP+ cells from FIG. 1H.

FIG. 1J shows a schematic view of an experimental set up. CD40 expressing Raji B cells are incubated with GFP viruses displaying either wild-type CD40 ligand (CD40L) or CD40L mutant (K142E, R202E) with decreased binding to its cognate receptor CD40.

FIG. 1K shows flow cytometry analysis of GFP signal in Raji B cells upon incubation with either wild-type CD40L or mutant CD40L displayed GFP viruses.

FIG. 1L is a Bar plot showing the percentage of GFP+ cells from FIG. 1K. P value was calculated by unpaired t-test. ***P<0.001.

FIG. 1M shows a schematic view of immunocapture assay. In this assay, magnetic beads were conjugated with anti-CD40L, anti-VSV-G, and IgG antibodies and then incubated with CD40L displayed viruses. Viruses that were immunocaptured were subjected to viral RNA isolation and qRT-PCR of CD40L.

FIG. 1N depicts a Bar plot showing the qRT-PCR result of enrichment of ENTER viruses by different antibody conjugated beads as in FIG. 1M. All p values were calculated by unpaired t-test. n.s. not significant p>0.05; *p<0.05; ***p<0.001.

FIGS. 2A-2F show how ENTER decodes interaction between MHC-peptides (pMHCs) with TCR.

FIG. 2A shows a schematic view of pMHC displayed viruses and flow cytometry analysis of GFP signal in specific TCR expressing Jurkat T cells upon incubation of pMHC displayed viruses. GFP fused viruses displaying either a 9-mer peptide (NY-ESO-1₁₅₇₋₁₆₅) presented by HLA-A0201(A2) allele or an 11-mer CMV peptide (pp65₃₆₃₋₃₇₃) presented by HLA-A0101(A1) allele are incubated with T cells expressing specific TCRs (e.g., NY-ESO-1₁₅₇₋₁₆₅-TCR or CMV-pp65₃₆₃₋₃₇₃-TCR) that recognize the cognate antigens. SP: signal peptide; Peptide: antigen peptide; B2M: Beta-2-Microglobulin.

FIG. 2B shows flow cytometry analysis of GFP signal in Jurkat T cells expressing specific TCRs (e.g., NY-ESO-1₁₅₇₋₁₆₅-TCR, CMV pp65₄₉₅₋₅₀₃₃-TCR, or Fluₘ1₍₅₈₋₆₆₎-TCR) upon incubation of viruses displaying various HLA-A2 presented peptides. HPV16 E7₈₂₋₉₁ peptide displayed viruses serve as a negative control.

FIG. 2C shows flow cytometry plots of NY-ESO-1 TCR-T cells upon incubation of 2×10⁸ ENTER viral particles displaying ny-eso-1₁₅₇₋₁₆₆ antigen (left) or with different amount of ny-eso-1₁₅₇₋₁₆₆ pMHC tetramers.

FIG. 2D shows a comparison of binding efficiency (GFP+%) of viruses displaying antigen variants with different TCR affinity. 1G4 wt-TCR T cells were incubated with ENTER displaying wild-type of mutant ny-eso-1₁₅₇₋₁₆₆ antigen variants for 2 hours. CMV-pp65₄₉₅₋₅₀₃-TCR T cells were used as negative control. The viral titers were normalized using the p24 protein level.

FIG. 2E shows a schematic view of experimental set up (top) and flow cytometry analysis (bottom) of TCR-T cell mixing experiment. Flu-m1₅₈₋₆₆-TCR T cells were labeled by CellTrace Violet dye and then mixed with CMV-pp65₄₉₅₋₅₀₃-TCR T cells at different ratio. Mixed T cell population was incubated with HLA-A2:ml displayed GFP viruses for 2 h and then subjected to flow cytometry. Representative flow cytometry plot showing 1:1000 mixing of two T cell population and GFP signal of T cells gated on Violet+ and Violet− population.

FIG. 2F depicts a bar plot showing the signal-to-noise ratio of ENTER in FIGS. 2E and 2J.

FIG. 2G shows a flow cytometry analysis of HLA-A2 and B2M surface expression in wild-type HEK293T, HLA-KO HEK293T, and HLA-A2 reconstituted HLA-KO HEK293T cells.

FIG. 2H shows flow cytometry plots of CMV-pp65₄₉₅₋₅₀₃-TCR T cells upon 2-hour incubation of HLA-A2-peptide displayed GFP viruses followed by staining of PE-tetramers. M1₍₅₈₋₆₆₎ (flu antigen) displayed viruses and M1₍₅₈₋₆₆₎ tetramers are negative controls.

FIG. 2I shows Histogram plots (left) and Bar plots (right) showing tetramer intensity and CD3 surface expression of CMV-pp65₄₉₅₋₅₀₃-TCR T cells.

FIG. 2J shows a schematic view of experimental design (top) and flow cytometry analysis (bottom) of T cell mixing experiments. Flu-m1₅₈₋₆₆-TCR T cells were labeled by Cell Trace Violet dye and then mixed with NY-ESO-1₁₅₇₋₁₆₅-TCR T cells at different ratio. Mixed T cell population was incubated with HLA-A2:m1 displayed GFP viruses for 2 h and then subjected to flow cytometry. Representative flow cytometry plot showing mixing of two T cell population and GFP signal of T cells gated on Violet+ and Violet− population.

FIG. 2K depict Bar plots showing sensitivity (left) and specificity (right) of ENTER from FIG. 2J.

FIGS. 3A-3F illustrate the optimization of ENTER to present intracellular antigens on viral surface and decode interaction between BCR with antigens. FIG. 3A shows a schematic view of experimental design. During the assembly and budding of lentiviruses, certain host cell surface proteins can be incorporated into the surface of viruses. TM domains of host proteins selected from literature are fused with a B cell epitope derived from intracellular antigen HPV16 L2. These HPV-epitope displayed GFP viruses are incubated with B cells expressing BCR targeting this HPV epitope (on-target) or B cells without any BCR expression (off-target).

FIG. 3B shows flow cytometry analysis of GFP signal in B cells incubated with GFP viruses displaying an HPV epitope fused with different TM domains. B cells without BCR expression serve as a negative control.

FIG. 3C depicts Bar plots showing the percentage of GFP+B cells from FIG. 3B.

FIG. 3D shows flow cytometry analysis of GFP signal in RBD-BCR+ B cells upon incubation of ENTER viruses displaying SARS-CoV-2 Spike RBD antigen or HPV L2 antigen as a negative control (left). Bar plot showing the frequency of GFP+ cells upon incubation of on-target or off-target ENTER viruses (right).

FIG. 3E shows a schematic view of experimental set up (top) and flow cytometry analysis (bottom) of B cell mixing experiment. RBD-BCR+ B cells were labeled by cell trace violet dye and then mixed with HPV-BCR+ B cells at different ratio. Mixed B cell population was incubated with GFP viruses displaying RBD antigen fused with ICAM1 TM domain and then subjected to flow cytometry. Representative flow cytometry plot showing 1:1000 mixing of two B cell population and GFP signal of B cells gated on violet+ and violet− population.

FIG. 3F depicts a Bar plot showing the signal-to-noise ratio of ENTER from FIGS. 3E and 3I.

FIGS. 3G-3P schematically summarize the results of experiments performed to decode antigen specificity of B cells and characterizing cargo delivery efficiency and specificity by ENTER. FIG. 3G depicts a flow cytometry analysis of GFP+ cells among HER2-BCR+ B cells that were incubated with HER2 displayed viruses or RBD displayed viruses.

FIG. 3H depicts a Bar plot showing the percentage of GFP+ cells as in FIG. 3G.

FIG. 3I shows a schematic view of experimental design (top) and flow cytometry analysis (bottom) of B cell mixing experiments. RBD-BCR+ B cells were labeled by Cell Trace violet and mixed with HPV-BCR+ B cells at different ratios. Mixed B cell population was incubated with GFP viruses displaying RBD antigen fused with ICAM1 TM domain and then subjected to flow cytometry. Representative flow cytometry plot showing mixing of two B cell population and GFP signal of B cells gated on Violet+ and Violet− population.

FIG. 3J depicts Bar plot showing sensitivity of ENTER from FIG. 3I.

FIG. 3K depicts Bar plot showing sensitivity of ENTER from FIG. 3I.

FIG. 3L depicts Bar plot showing the delivery efficiency of viruses with wild-type VSV-G in cell types with different antigen specificity. The p values were calculated by unpaired t-test. n.s. not significant P>0.05.

FIG. 3M depicts a flow cytometry analysis of cargo delivery in T cell mixing experiment. mScarlet expressed NY-ESO-1 TCR+ T cells were mixed with CMV-pp65 TCR T cells and then infected with ENTER viruses displaying pp65₄₉₅₋₅₀₃ antigen and carrying transgene containing HSV-TK and GFP.

FIG. 3N depicts a flow cytometry analysis of cargo delivery in B cell mixing experiment. mScarlet expressed RBD BCR+ B cells were mixed with HER2 BCR+ B cells and then infected with ENTER viruses displaying HER2 antigen and carrying transgene containing HSV-TK and GFP.

FIG. 3O depicts a Histogram plot showing the surface expression of FAS in T cells transduced with diverse FAS shRNAs or control shRNA.

FIG. 3P depicts a representative flow plot showing the Annexin V and 7-AAD gating of T cells upon anti-FAS induced apoptosis and cell death.

FIGS. 4A-4M schematically summarize the results of experiments performed to demonstrate that ENTER permits selective depletion or expansion of antigen-specific T or antigen-specific B cells. FIG. 4A is a schematic view of cargo delivery in antigen-specific T cells (left). CMV-pp65 TCR+ T cells or NY-ESO-1 TCR+ T cells were individually infected with ENTER viruses that display pp65₄₉₅₋₅₀₃ and carry GFP transgene as a cargo. Representative histogram plot showing GFP expression between CMV-pp65 TCR+ T cells and NY-ESO-1 TCR+ T cells after 2 days infection.

FIG. 4B is a Bar plot showing the percentage of GFP+ cells as in FIG. 4A.

FIG. 4C is a schematic view of cargo delivery in antigen-specific B cells (left). HER2 BCR+ B cells or RBD BCR+ B cells were individually infected with ENTER viruses that display HER2 and carry GFP transgene as a cargo. Representative histogram plot showing GFP expression between HER2 BCR+ B cells and RBD BCR+ B cells after 2 days infection.

FIG. 4D is a Bar plot showing the percentage of GFP+ cells as in FIG. 4C.

FIG. 4E is a schematic view of suicide gene delivery in a pool of different antigen-specific T cells. CMV-pp65 TCR+ T cells and NY-ESO-1 TCR+ T cells expressing mScarlet were mixed together and then infected of ENTER viruses that display pp65₄₉₅₋₅₀₃ and carry a herpes simplex virus thymidine kinase (HSV-TK) transgene. After 2 days of infection, ganciclovir (GCV) drug was added to kill HSV-TK expressing cells and cell survival was monitored for 4 days.

FIG. 4F is a Bar plot showing the number of live T cells at day 4 post GCV treatment.

FIG. 4G schematically summarizes the results of a kinetics analysis of fold enrichment between the number of CMV-pp65 TCR+ T cells versus that of NY-ESO-1 TCR+ T cells that were either infected with ENTER carrying GFP gene or ENTER carrying HSV-TK gene upon GCV drug treatment.

FIG. 4H is a schematic view of suicide gene delivery in a pool of different antigen-specific B cells. HER2 BCR+ B cells and RBD BCR+ B cells expressing mScarlet were mixed together and then infected of ENTER viruses that display pp65₄₉₅₋₅₀₃ and carry HSV-TK transgene. After 2 days of infection, GCV drug was added to kill HSV-TK expressing cells and cell survival was monitored for 4 days.

FIG. 4I is a Bar plot showing the number of live B cells at day 4 post GCV treatment.

FIG. 4J schematically summarizes the results of a kinetics analysis of fold enrichment between the number of HER2 BCR+ B cells versus that of RBD BCR+ B cells that were either infected with ENTER carrying GFP gene or ENTER carrying HSV-TK gene upon GCV drug treatment.

FIG. 4K is a schematic view of shRNA delivery in a pool of different antigen-specific T cells. CMV-pp65 TCR+ T cells and NY-ESO-1 TCR+ T cells expressing mScarlet were mixed together and then infected of ENTER viruses that display pp65₄₉₅₋₅₀₃ and carry FAS shRNA or control shRNA. Anti-FAS antibody was added to induce apoptosis.

FIG. 4L is a Bar plot showing the surface expression of FAS in off-target NY-ESO-1 TCR+ T cells (uninfected group), and on-target CMV-pp65 TCR+ T cells transduced with control shRNA or FAS shRNA.

FIG. 4M is a Bar plot showing the CMV-pp65 TCR+ T cells/NY-ESO-1 TCR+ T cells fold enrichment that is normalized by shCtrl group for the live cells (gated on Annexin V and 7-AAD double negative) as in FIG. 4K. P-values are calculated by unpaired t-test. ****P<0.0001; ***P<0.001; **P<0.01; *P<0.05; n.s. P≥0.05.

FIGS. 4N-4W schematically summarize the results of experiments performed to optimize ENTER to detect antigen-specific primary human T cells.

FIG. 4N is a flow cytometry analysis of GFP signal in T cells expressing TCRs (NY-ESO-1 TCR or CMV pp65-TCR) upon incubation with ny-eso-1₁₅₇₋₁₆₅ antigen peptide displayed GFP viruses whose viral RNA are either intact or inserted with sequencing capture tag.

FIG. 4O is a Bar plot showing the percentage of GFP+ cells from FIG. 4N. P value was calculated by unpaired t-test. n.s. P>0.05.

FIG. 4P is a schematic view of experimental design. Donor isolated T cells were incubated with pp65₄₉₅₋₅₀₃ displayed viruses carrying either GFP or mNeon fluorescence proteins and then stained with pp65₄₉₅₋₅₀₃ tetramer and other antibodies followed by flow cytometry. We first gated on CD3+ CD8+ T cells to measure pp65-specific T cells using tetramer as a positive control, and then monitored GFP signals in pp65 tetramer+ T cells.

FIG. 4Q is a flow cytometry analysis of tetramer and GFP signal in primary human T cells from donors with CMV infection as in FIG. 4P.

FIG. 4R is a Bar plot showing the percentage of GFP+ and GFP− cells among pp65 tetramer+ T cells from FIG. 4Q. P value was calculated by unpaired t-test. *p<0.05.

FIG. 4S is a representative flow cytometry plot showing the GFP+ cells among pp65 tetramer+ T cells after incubation with pp65₄₉₅₋₅₀₃ viruses or negative control viruses (ny-eso-1₁₅₇₋₁₆₅).

FIG. 4T is a schematic view of experimental design. PBMCs were isolated from CMV seropositive HLA-A2+ donors and cultured with a pool of twelve different CMV antigen peptides (10 μg/mL) for 10 days. Primary T cells prior or post peptide-induced expansion were incubated with pp65 antigen displayed mNeon viruses for 2 hours and then stained with antibodies followed by flow cytometry.

FIG. 4U is a representative flow cytometry plot showing co-staining of pp65₄₉₅₋₅₀₃ tetramer and pp65₄₉₅₋₅₀₃ displayed mNeon viruses in pp65₄₉₅₋₅₀₃ peptide enriched T cells post 15 days expansion. These T cells are gated on live CD8+ CD3+ T cells.

FIG. 4V is a representative flow cytometry plot showing the percentage of GFP+ T cells (stained by 12 pooled HLA-A2:CMV-antigen mNeon viruses) from 4 different CMV seropositive donors prior or post pooled CMV peptide-induced expansion as in FIG. 4T.

FIG. 4W is a MA plot showing the bulk RNA-seq analysis of CMV-pp65 TCR T cells incubated with pp65₄₉₅₋₅₀₃ tetramer or pp65₄₉₅₋₅₀₃ displayed ENTER viruses. Genes with log 2 fold change and adjusted p value <0.01 are highlighted in red.

FIGS. 5A-5F schematically illustrate an ENTER-seq for massively parallel measurement of antigen peptide sequence, TCR sequence, and transcriptome. FIG. 5A shows a schematic view of ENTER-seq workflow. A library of pooled viruses displaying individual pMHCs was incubated with T cells for 2 hours. GFP+ T cells are sorted for droplet-based single cell genomics profiling (e.g. 10× Genomics 5′ kit for gene expression and V(D)J immune profiling).

FIG. 5B shows a viral RNA engineering strategy for droplet-based single cell capture. 10× Genomics capture tag is inserted in the linker region between B2M and HLA-A2. 10× Genomics PCR handle is inserted after CMV promoter. CMV: CMV promoter; SP: signal peptide sequence; Peptide: antigen peptide; B2M: Beta-2-Microglobulin; MHC Class I: HLA-A0201 allele; LTR: long terminal repeat; TSO: template switching oligo sequence.

FIG. 5C shows a schematic view of T cell mixing experiment for ENTER-seq.

FIG. 5D shows number of Pp65₍₄₉₅₋₅₀₃₎-TCR T cells UMI counts (x-axis) and NY-ESO-1₁₅₇₋₁₆₅-TCR UMI counts (y-axis) associated with each cell barcode (dot). The colors are assigned as NY-ESO-1₁₅₇₋₁₆₅-TCR+ T cells (light blue), Pp65₍₄₉₅₋₅₀₃₎-TCR+ T cells (red), and doublets (green, with both NY-ESO-1₁₅₇₋₁₆₅-TCR and Pp65₍₄₉₅₋₅₀₃₎-TCR).

FIG. 5E shows scatter plots of TCR UMI counts after doublet removal, colored by enrichment ratio of pp65₍₄₉₅₋₅₀₃₎-antigen UMI count among total UMI counts (left), and enrichment ratio of nyeso₁₅₇₋₁₆₅-antigen UMI count among total UMI counts (right).

FIG. 5F shows number of pp65₍₄₉₅₋₅₀₃₎-antigen UMI counts (x-axis) and nyeso₁₅₇₋₁₆₅-antigen UMI counts (y-axis) associated with each cell barcode (dot) after doublet removal. The colors are assigned as NY-ESO-1₁₅₇₋₁₆₅-TCR+ T cells (light blue) and Pp65₍₄₉₅₋₅₀₃₎-TCR+ T cells (red).

FIGS. 5G-5N schematically summarize the results of experiments performed for the characterization of T cell subsets by ENTER-seq. FIG. 5G is a UMAP plots showing CMV-specific T cells (yellow) and bystander T cells (blue) via clustering before or after removal of ENTER-induced gene signature.

FIG. 5H is a UMAP plot showing 10 clusters of human CD8 T cell subsets.

FIG. 5I depicts UMAP plots showing amount of surface protein CD127 from CITE-seq, and expression of genes for naïve T cells (CCR7, SELL, and LEF1).

FIG. 5J depicts UMAP plots showing gene expression of cytolytic molecules.

FIG. 5K depicts UMAP plots showing gene expression of markers for MAIT cells.

FIG. 5L depicts UMAP plots showing expression of marker genes for each subset/cluster.

FIG. 5M is a UMAP plot showing donor origin.

FIG. 5N shows fraction of T cell subsets from 10 clusters in CMV antigen-specific T cells (ENTER+) and bystander T cells (ENTER−), separated by donor origin.

FIGS. 6A-6I schematically summarize the results of ENTER-seq of ex vivo expanded CMV-specific primary T cells. FIG. 6A shows a schematic view of CMV antigen peptide induced T cell expansion and ENTER-seq workflow.

FIG. 6B is a UMAP plot showing cells with (ENTER+, colored in yellow) or without (ENTER−, colored in blue) CMV antigen displayed viruses binding.

FIG. 6C depicts UMAP plots showing CITE-seq of surface protein expression of CD45RA (naïve marker) and CD45RO (memory marker).

FIG. 6D is a UMAP plot showing 10 clusters of human CD8+ T cell subsets labeled in different colors.

FIG. 6E is a Bar plot showing the number of CMV antigen-specific T cells that recognize specific CMV antigen epitope in donor #1 (labeled in black) and donor #2 (labeled in gray).

FIG. 6F depicts UMAP plots showing the amount of CMV antigen epitopes per cell for the top 3 CMV antigen epitopes identified from FIG. 6E.

FIG. 6G is a heatmap showing column scaled expression of representative genes associated with effector function or Treg signature among different CMV antigen-specific T cells.

FIG. 6H is a UMAP plot (left) showing the clonal expansion size of CMV antigen-specific T cells colored by the number of cells in each clonotype. Violin plot (right) showing the distribution of clone size in different CMV antigen-specific T cells colored by antigen epitopes.

FIG. 6I is a Violin plots showing expression of cytokines and transcription factors in pp65₄₉₅₋₅₀₃-specific TCR clones, separated and colored by CDR3 clones. A simplified model (right).

FIGS. 6J-6P summarize the results of a TCR clonotype analysis of ex vivo expanded CMV-specific T cells. FIG. 6J depicts UMAP plots showing the clonal expansion size of CMV antigen-specific T cells (ENTER+) and bystander T cells (ENTER−).

FIG. 6K: TCR clonotypes of CMV antigen-specific T cells colored by donor. Each circle represents a clonotype with identical CDR3 nucleotide sequences. The size of circle represents the number of cells in each clonotype.

FIG. 6L depicts scatter plots showing the correlation of clonal expansion size with cytotoxic gene score.

FIG. 6M depicts scatter plots showing the correlation of clonal expansion size with exhaustion gene score or activation gene score.

FIG. 6N is a summary table of pp65₄₉₅₋₅₀₃-specific TCR clones showing convergent TCR clonotypes with identical CDR3 amino acid sequences.

FIG. 6O is a summary tables of US8₇₄₋₈₂- and UL100₂₀₀₋₂₀₈-specific TCR clones.

FIG. 6P depicts fraction of T cell subsets in pp65₄₉₅₋₅₀₃-specific TCR clones, separated by CDR3 clones and colored by 10 clusters. Coefficient r and p values in FIGS. 6L-6M are calculated by Pearson correlation.

FIGS. 7A-7K schematically summarize the results of ENTER-seq of primary CMV-specific T cells isolated directly (e.g., without in vitro expansion) from CMV seropositive patient blood. FIG. 7A depicts a schematic view of isolation of primary T cells from patient blood and ENTER-seq workflow.

FIG. 7B is a UMAP plot (left) showing cells with (ENTER+, colored in yellow) or without (ENTER−, colored in blue) CMV antigen displayed viruses binding. UMAP plot (right) showing 13 clusters of human CD8+ T cell subsets labeled in different colors.

FIG. 7C depicts UMAP plots showing the surface protein expression of CD45RA and CD45RO from CITE-seq and expression of representative genes.

FIG. 7D is a heatmap showing the scaled z score of expression of genes associated diverse functions (e.g., type-I IFN, cytotoxicity, etc.) across different clusters.

FIG. 7E is a UMAP plot showing the CMV antigen specificity colored by antigen epitope.

FIG. 7F is a UMAP plot (left) showing the clonal expansion size of CMV antigen-specific T cells colored by the number of cells in each clonotype.

FIG. 7G is a Violin plot showing the number of pMHC bound per cell in T cells with different clone size. P value was calculated by Mann-Whitney test. n.s. p≥0.05; *p<0.05; **p<0.01, ***p<0.001, ****p<0.0001.

FIG. 7H is a CITE-seq density plot showing surface expression of CD45RA and CD45RO in pp65-specific T cells from donor #1 (colored by orange) and donor #2 (colored by blue) before and after peptide-induced expansion.

FIG. 7I depicts flow cytometry plots showing CD45RA and CD45RO in pp65-specific T cells from donor #1 and donor #2 before and after peptide-induced expansion.

FIG. 7J is a density plot showing the distribution of type-I IFN ISG gene score and cytotoxicity gene score prior and post peptide-induced expansion in top 3 TCR clones of pp65-specific T cells (left). Density plot showing the expression of IL13 and EOMES prior and post peptide-induced expansion in top 3 TCR clones of pp65-specific T cells (right).

FIG. 7K is a proposed model of phenotypic transition of CMV-specific T cells upon ex vivo expansion.

FIGS. 7L-7T schematically summarize the results of intra-clonal phenotypic diversity of CMV-specific T cells isolated directly from patient blood. FIG. 7L depicts UMAP plots showing the expression of key genes associated with diverse functions (type-I IFN ISG, cytotoxicity, chemokine, and transcription factors).

FIG. 7M depicts UMAP plots showing the subset clustering of primary T cells directly isolated from patient blood sample before and after removal of ENTER-induced gene signature.

FIG. 7N depicts UMAP embedding density plots showing the density of CMV antigen-specific T cells for different CMV antigen epitopes.

FIG. 7O is a Bar plot showing the percentage of US8₇₄₋₈₂-specific T cells with shared TCR before and after peptide induced expansion.

FIG. 7P depicts the number of US8₇₄₋₈₂-specific T cells isolated from fresh PBMC before expansion, separated by donor and colored by TCR CDR3 sequence. TCR sequence shared before and after expansion are circled in black.

FIG. 7Q is a summary table of top TCR clones of US8₇₄₋₈₂-specific T cells (identified from E) with cell number and frequency metrics before and after expansion.

FIG. 7R depicts the number of T cell subsets in pp65₄₉₅₋₅₀₃-specific TCR clones, separated by CDR3 clones and colored by 13 clusters.

FIG. 7S is a heatmap showing the expression of genes associated with diverse functions among different TCR clone-specific T cells.

FIG. 7T depicts density plots showing the expression of IL13 and IL4 in different TCR clone-specific T cells before and after peptide-induced expansion.

### DETAILED DESCRIPTION OF THE DISCLOSURE

The present disclosure generally relates to systems, compositions and methods for the identification of receptor-ligand pairing including MHC-peptides/T-cell receptors and antigen/B-cell receptor (antibody) pairs and for decoding, e.g., displaying ligand proteins, delivering payloads, and recording receptor specificity. Some embodiments of the disclosure relate to decoding interactions between T-cell receptors and MHC peptides, between antibodies and antigens, or between B-cell receptors and B cell antigens, including intracellular/secreted epitopes/cell-surface antigen epitopes, as well as other ligand-receptors (e.g., CD40 ligand vs CD40). The disclosure also relates to compositions and methods for protein or nucleic acid delivery into user-defined target cells. In particular, some embodiments of the disclosure relate to a modular viral display and delivery platform to decode ligand-receptor interactions, deliver cargos in target cells, and connect ligand-receptor interactions with cellular state. In some embodiments, lentiviruses can be engineered at multiple levels including (1) displaying user-defined ligand proteins on the viral surface; (2) engineering a fusogen to achieve receptor-specific cell entry of cognate ligand displayed viruses; (3) carrying fluorescent proteins to track engineered viruses; (4) delivering cargos upon paired ligand-receptor recognition; and (5) modifying viral RNA to record ligand information by sequencing. This technology is termed “ENTER” for “lentiviral-mediated cell entry by engineered ligand-receptor interaction), which can systematically deorphanize pairs of interactions including TCR-pMHC, antibody-antigen, costimulatory ligand-receptors, and B cell antigen-BCR. In some embodiments, ENTER can permit gene delivery in a receptor-specific manner, allowing for the selective manipulation of cellular behavior in antigen-specific T and B cells. In some embodiments, ENTER can be combined ENTER with droplet-based single-cell genomics profiling (ENTER-seq) to measure antigen specificity, TCR repertoire, gene expression and surface protein landscape in individual human primary T cells.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

### II. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a” “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “exogenous” refers to any material introduced from or produced outside an organism, cell, tissue or system.

The term “linker”, also referred to as a “spacer” or “spacer domain” as used herein, refers to an amino acid or sequence of amino acids that that is optionally located between two amino acid sequences in a fusion protein of the invention.

The term “biological sample” or “sample” refers to any solid or liquid sample isolated from an individual or a subject. For example, it can refer to any solid (e.g., tissue sample) or liquid sample (e.g., blood) isolated from an animal (e.g., human), such as, without limitations, a biopsy material (e.g., solid tissue sample), or blood (e.g., whole blood). Such sample can be, for example, fresh, fixed (e.g., formalin-, alcohol- or acetone-fixed), paraffin-embedded or frozen prior to an analysis. In some embodiments, the biological sample is obtained from a tumor (e.g., a pancreatic cancer). A “test biological sample” is the biological sample that has been the subject of analysis, monitoring, or observation. A “reference biological sample,” containing the same type of biological sample (e.g., the same type of tissues or cells), is a control for the test biological sample.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid constructs described herein (e.g., lentiviral vectors) or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker). In some embodiments, the operably linked segments, portions, regions, and domains of the polypeptides described herein are fused in-frame to one another.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

A population of cells as described herein may be any mammalian cell population. In some embodiments, a population of cells is a population of human, mouse, rat, or non-human primate cells. In some embodiments, a population of cells is a somatic cell population or a reproductive cell population. In some embodiments, a population of cells comprises antigen-specific cells (e.g., cells that binds to a specific antigen). In some embodiments, a population of antigen-specific cells comprises immune cells. In some embodiments, a population of antigen-specific cells comprises B cells and/or T cells. In some embodiments, a population of cells comprises a homogenous population of cells. In some embodiments, a population of cells comprises a heterogeneous population of cells. In some embodiments, a population of cells is a population of cells isolated from a subject. A subject may be a human subject (e.g., a human subject suffering from a disease), a mouse subject, a rat subject, or a non-human primate subject. In some embodiments, a population of cells is isolated from the blood or a tumor of a subject.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Furthermore, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

### III. Compositions of the Disclosure

### Engineered Lentiviruses

Provided herein are compositions comprising a lentivirus engineered so that it can a) display a specific ligand on the cell surface, b) have a mutated fusogen that allows the virus to fuse with and enter only host cells having a receptor that naturally pairs with the ligand, c) deliver a reporter to the host cells and d) be tagged, on the viral RNA, to allow single cell sequencing

In some embodiments, the lentivirus can be further engineered to comprise a defective integrase so that the viral RNA cannot integrate into the genome of the host cell, thereby avoiding integration-induced mutagenesis of the host genome.

### Ligands

In some embodiments, the engineered lentiviruses of the present disclosure comprise one or more user-defined ligands displayed on the viral cell surface. In some embodiments, the ligand is heterologous relative to the lentivirus displaying the ligand on its surface, e.g., the ligand is from a heterologous source, such as from another cell or another virus. Non-limiting examples of suitable ligand types include cell surface receptors, adhesion proteins, glycoproteins, carbohydrates, lipids, glycolipids, lipoproteins, and lipopolysaccharides that are surface-bound, integrins, mucins, and lectins. In some embodiments, the ligands can be or comprise proteins. In some embodiments, the ligands can be or comprise epitopes. One skilled in the art will understand that the term “epitope” refers to an antigenic determinant that interacts with a specific antigen-binding site of an antigen-binding polypeptide, e.g., a variable region of an antibody molecule, known as a paratope. A single antigen can have more than one epitope. Thus, different antibodies may bind to different areas on an antigen and may have different biological effects. The term “epitope” also refers to a site on an antigen to which B cells respond. It also refers to a region of an antigen that is bound by an antibody. Epitopes can be defined as structural or functional. Functional epitopes are generally a subset of the structural epitopes and have those residues that directly contribute to the affinity of the interaction. Epitopes can be linear or conformational, that is, composed of non-linear amino acids. In certain embodiments, epitopes can include determinants that are chemically active surface groupings of molecules such as amino acids, sugar side chains, phosphoryl groups, or sulfonyl groups, and, in certain embodiments, can have specific three-dimensional structural characteristics, and/or specific charge characteristics. In some embodiments, the ligands can be or comprise cell surface proteins or intracellular proteins or parts thereof. In some embodiments, the ligands can be or comprise MHC peptides, antibodies, intracellular antigens, secreted proteins, or other forms of proteins or peptides.

In some embodiments, if the ligand does not contain a native transmembrane domain, a signal peptide and a transmembrane domain is added to fuse with the ligand. Accordingly, in some embodiments, the transmembrane domain is operably linked to a transmembrane domain. In some embodiments, the TM domains may facilitate the efficiency in virus carry-over. In some embodiments, the transmembrane domain is a heterologous transmembrane domain. In some embodiments, the transmembrane domain is a heterologous transmembrane derived from HLA-DRA, HLA-DRB, HLA-A2, ICAM1, CD43, CD162, CD62L, CD49d, or LFA-1. In some embodiments, the TM is replaced with an optimized TM (such as, for example, TM from ICAM1, PDGFR).

In some embodiments, the ligand can be engineered to first be displayed on the surface of a cell line that is suitable to produce lentivirus (for example, HEK 293T, a common type of cell to produce lentivirus). The ligand can get carried over on to the virus surface during virus budding to produce virus particles.

In some embodiments, the engineered lentivirus of the disclosure comprises a defective integrase protein.

### Reporter Genes

In some embodiments, the lentiviruses described herein may comprise a reporter (e.g., a reporter protein). In some embodiments, the lentiviruses comprises a nucleic acid encoding a reporter (e.g., a reporter protein). As used herein, a reporter is generally a protein or gene that can be detected when expressed in a retrovirus and/or target cell. In some embodiments, the presence or absence of a reporter in a target cell or a subset of a target cells in a population of cells allows for the ability to sort cells (e.g., using flow cytometry and/or fluorescence-activated cell sorting).

In some embodiments, a reporter is a fluorescent protein. A fluorescent protein may be a green fluorescent protein (GFP), yellow fluorescent protein (YFP), red fluorescent protein (RFP). Exemplary fluorescent proteins may be as described in U.S. Pat. No. 7,060,869. The engineered lentiviral particles displaying specific ligands deliver fluorescent protein into target cells upon cognate receptor-ligand interaction.

In some embodiments, the reporter is mNeon, a monomeric green fluorescence protein that is substantially brighter than GFP.

In some embodiments, the reporter can be operably linked, e.g., fused to a viral structural gene. In some embodiments, the structural gene can be a nucleocapsid protein (NC) or a Gag protein. A person of skill in the art would recognize that other structural genes may be used without deviating from the teachings of the present disclosure.

In some embodiments, the barcoded RNA is encapsulated in viral particles, e.g., those produced by the engineered lentiviruses. In some embodiments, the RNA encodes the ligand. In some embodiments, the RNA encodes a gene of interest to be delivered into target cells, e.g., host cells. In some embodiments, the RNA is read out by a next-generation sequencing technology. In some embodiments, the RNA comprises a capture sequence.

### Fusogens

The term “fusogen” or “fusogenic molecule” as used herein generally refers to any molecule that can facilitate, catalyze, or trigger membrane fusion when present on the surface of a virus. Non-limiting examples of fusogens suitable for the compositions and methods of the disclosure include fusogens derived from viruses or fusogens endogenously expressed in a host cell, e.g., mammalian cell (e.g., human cell). In some embodiments, the fusogens of the disclosure are glycoproteins. In some embodiments, the fusogens of the disclosure are viral glycoproteins. Exemplary viral fusogens suitable for the compositions and methods disclosed herein include those belonging to Classes I, II, and III of viral fusion proteins, which are produced by enveloped viruses to facilitate virus-host membrane fusion. Addition information in this regard can be found in, e.g., Vance T. D. R and Lee J. E. (Curr. Biol. July 6, 30(13), R750-R754, 2020), which is incorporated herein by reference. In some embodiments, the fusogens of the disclosure belong to Class I viral fusion proteins, i.e., those capable of forming form coiled-coil trimers, which include but are not limited to, from influenza viruses, coronaviruses, HIV, and Ebola virus. In some embodiments, the fusogens of the disclosure belong to Class II viral fusion proteins, i.e., those capable of transitioning from dimers to trimers during fusion, producing an elongated ectodomain heavily composed of β sheets that settles into a hairpin trimer after fusion. Suitable Class II viral fusion proteins include but are not limited to, those from Dengue fever virus, West Nile virus, Zika virus, and tick-borne encephalitis virus. In some embodiments, the fusogens of the disclosure belong to Class III viral fusion proteins, i.e., those capable of combining elements from the former two classes, taking on a post-fusion conformation that contains both a coiled-coil trimerization region similar to Class I, and an elongated trimer of hairpins as in Class II. Suitable Class III viral fusion proteins include but are not limited to, those from vesicular stomatitis virus (VSV), herpes simplex virus 1 (HSV1), rabies virus. In some embodiments, the fusogen of the compositions and methods disclosure herein is or comprises a vesicular stomatitis virus G (VSV-G) protein. In some embodiments, the fusogen of the compositions and methods disclosure herein is or comprises a viral fusion protein from measles virus, Sindbis virus, Baboon endogenous retrovirus (BaEV), murine leukemia virus, rabies virus, Nipah virus, RD 114 retrovirus, Gibbon-ape leukemia virus (GALV), Tupaia paramyxovirus (TPMV), or human endogenous retrovirus (HERV) such as ERVW-1 (e.g., Syncytin-1).

In some embodiments, the engineered lentiviruses can comprise modified fusogens to, e.g., facilitate the fusion of the virus with cell membranes. The fusogen can be modified so that it facilitates fusion of the virus to cell membranes without the need for a viral surface glycoprotein. In some embodiments, the fusogen comprises a modified vesicular stomatitis virus G (VSV-G) viral envelope protein. In some embodiments, the VSV-G polypeptide is or comprises the sequence of SEQ ID NO: 56. In some embodiments, the modified VSV-G viral envelope protein comprises one or more substitutions, for example, substitutions that abolish the binding of VSV-G with the cellular receptor. In some embodiments, the VSV-G envelope protein may include one or more amino acid substitutions at a position corresponding to any one of the positions H8, K47, Y209, and R354 of the VSV-G polypeptide. It is within the knowledge of the skilled person to know how to align amino acid sequences, e.g., sequences of multiple VSV-G polypeptides, in order to determine which amino acid in a particular position referred to herein “corresponds to” in another VSV-G amino acid sequence not listed herein. Thus, the term “position corresponding to” as used herein, is well-known within the art.

The modified VSV-G viral envelope protein disclosed herein can also include conservative modifications and substitutions at other positions of VSV-G (e.g., those that abolish the binding of VSV-G with the cellular receptor). Such conservative substitutions include those described by Dayhoff 1978, supra, and by Argos 1989, supra. For example, amino acids belonging to one of the following groups represent conservative changes: Group I: Ala, Pro, Gly, Gln, Asn, Ser, Thr; Group II Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. In some embodiments, the amino acid substitution(s) in the amino acid sequence of the modified VSV-G viral envelope protein disclosed herein is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution, and combinations of any thereof. In some embodiments, the amino acid substitutions(s) in the amino acid sequence of the modified VSV-G viral envelope protein disclosed herein includes an alanine substitution.

In some embodiments, the modified VSV-G viral envelope protein comprises an amino acid substitution corresponding to K47Q substitution or a R354A substitution of the sequence of SEQ ID NO: 56. In some embodiments, the modified VSV-G viral envelope protein comprises a K47Q and a R354A substitution. As described above, other viral fusogens that are able to fuse viral particles with cell membrane may also be suitably used with ENTER.

### Single Cell Sequencing

ENTER can be adapted to couple with any single cell methods that use whole cell as input and contains a step of reverse transcription. For example, it is compatible with published or commercial single cell sequencing technology such as any single cell RNA-seq (10× Genomics or others) that include 5′ or 3′ approach, scTCR/BCR-seq (10× Genomics) to identify immune VDJ recombination in B-cells and T-cells, CITE-seq/ECCITE to identify surface markers with barcoded antibodies, single cell CRISPR perturb-seq to perturb genes with CRISPR coupled with single cell's transcriptome.

### IV. Methods of the Disclosures

### Methods for Identifying a Ligand-Receptor Pair

Provided herein are methods for identifying a ligand-receptor pair, by (i) providing at least one engineered lentivirus as disclosed herein, (ii) combining the lentivirus with a population of cells; and (iii) sorting the population of cells based on the presence of the reporter gene, thereby identifying a ligand-receptor pair. In some embodiments, the engineered lentivirus comprises a ligand displayed on a surface of the lentivirus wherein the ligand is heterologous relative to the lentivirus; a fusogen comprising a modified viral envelope protein, wherein the fusogen is capable of fusing the lentivirus to a host cell (e.g., capable of facilitating, catalyzing, or triggering fusion of the lentivirus with the host cell), wherein the host cell comprises an endogenous receptor for the ligand, a reporter protein operably linked, e.g., fused to a lentiviral structural protein and a barcoded RNA.

The lentivirus and a population of cells can be combined for a defined period of time. In some embodiments, a period of time may be measured in seconds, minutes, hours or days. In some embodiments, period of time is 0-30 seconds, 15-45 seconds, 30-60 seconds, 45-90 seconds, 60-90 seconds, or 60-120 seconds. In some embodiments, the virus and a population of cells are combined and in contact for 0-30 seconds, 15-45 seconds, 30-60 seconds, 45-90 seconds, 60-90 seconds, or 60-120 seconds. In some embodiments, period of time is 1-2 minutes, 1-5 minutes, 1-10 minutes, 2-10 minutes, 5-10 minutes, 5-20 minutes, 10-20 minutes, 25-30 minutes, 25-60 minutes, 30-45 minutes, 30-40 minutes, 40-60 minutes, 50-70 minutes, or 60-120 minutes. In some embodiments, a retrovirus and a population of cells are combined and in contact for 1-2 minutes, 1-5 minutes, 1-10 minutes, 2-10 minutes, 5-10 minutes, 5-20 minutes, 10-20 minutes, 25-30 minutes, 25-60 minutes, 30-45 minutes, 30-40 minutes, 40-60 minutes, 50-70 minutes, or 60-120 minutes. In some embodiments, a period of time is 1-2 hours, 1-5 hours, 1-3 hours, 2-5 hours, 3-6 hours, 3-12 hours, 6-12 hours, 12-18 hours, 12-24 hours, 15-30 hours, 18-24 hours, 24-48 hours, 24-36 hours, or 36-50 hours. In some embodiments, a virus and a population of cells are combined and in contact for 1-2 hours, 1-5 hours, 1-3 hours, 2-5 hours, 3-6 hours, 3-12 hours, 6-12 hours, 12-18 hours, 12-24 hours, 15-30 hours, 18-24 hours, 24-48 hours, 24-36 hours, or 36-50 hours. In some embodiments, a period of time is 1, 2, 3, 4, 5, 6, 7, 8, 9, 10, or 5-15 days. In some embodiments, a virus and a population of cells are combined and in contact for 1, 2, 3, 4, 5, 6, 7, 8, 9, 10, or 5-15 days. In one aspect, the method comprises combining the lentivirus with cells, for 2 hours to identify ligand-receptor pairs.

In some embodiments, the lentivirus and the population of cells can be combined at a temperature ranging from 4° C. to 42° C., 4° C. to 8° C., 4° C. to 10° C., 8° C. to 150° C., 100° C. to 200° C., 180° C. to 230° C., 200° C. to 300° C., 250° C. to 350° C., 300° C. to 400° C., or 370° C. to 420° C.

In some embodiments, the population of cells and the virus can be incubated at 37° C. for about 2 hours to identify ligand-receptor pairs. However, modifications are within the scope of the disclosure.

Methods for Identifying a T-Cell Receptor and Paired pMHC

Further, provided herein are methods for identifying a T-cell receptor and paired pMHC, comprising (i) providing an engineered lentivirus as disclosed herein, (ii) combining the lentivirus with the population of cells; and (iii) sorting the population of cells based on the presence of the reporter thereby identifying the T-cell receptor. In some embodiments, the engineered lentivirus comprises a pMHC displayed on virus surface, a fusogen comprising a modified VSV-G viral envelope protein wherein the fusogen is capable of fusing the lentivirus to a host cell, wherein the host cell comprises a T-cell receptor for the pMHC, a reporter transgene operably linked, e.g., fused to a lentivirus structural protein, and a barcoded RNA. In some embodiments, the method provides a mixture of virus displaying different MHC peptide. (e.g., a pool of MHC peptides). In some embodiments, the T-cells can be a population of cell lines. In another embodiment the cells are human primary T-cells.

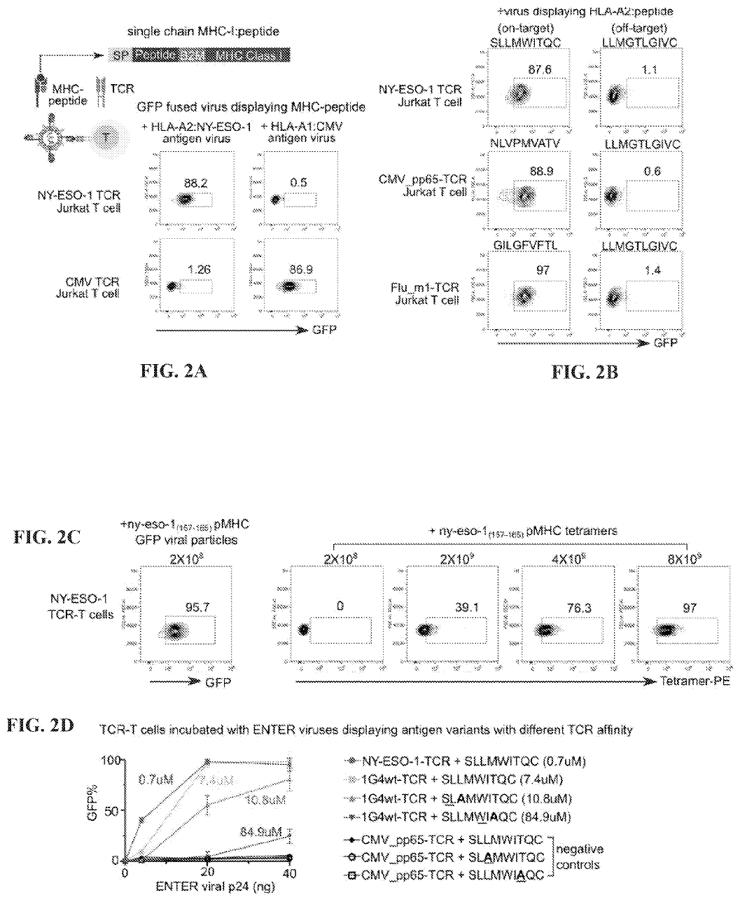

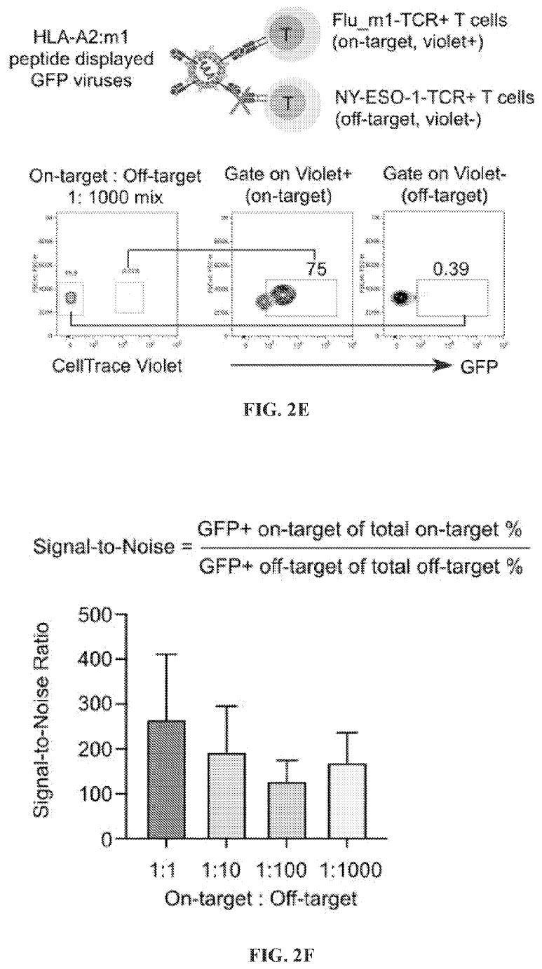

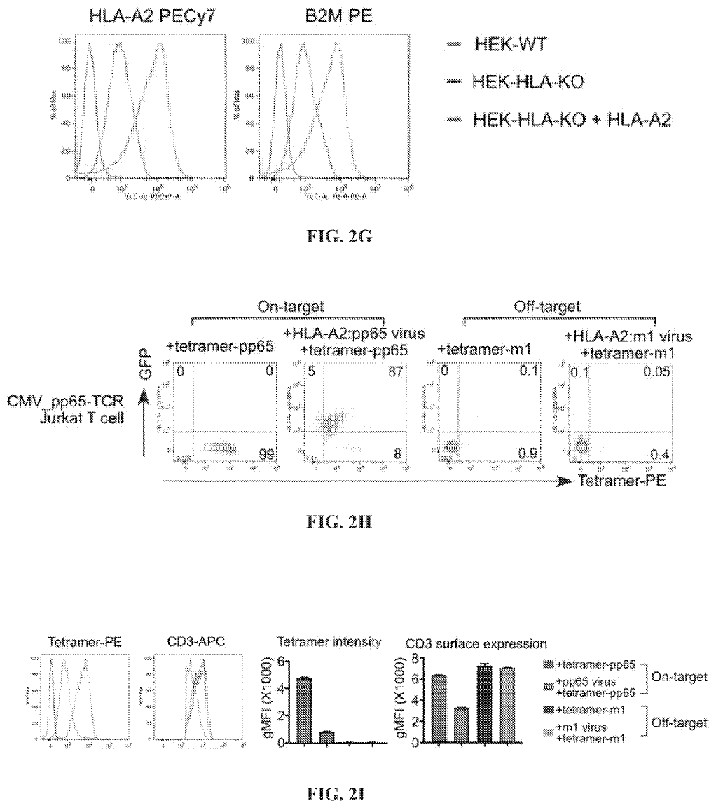

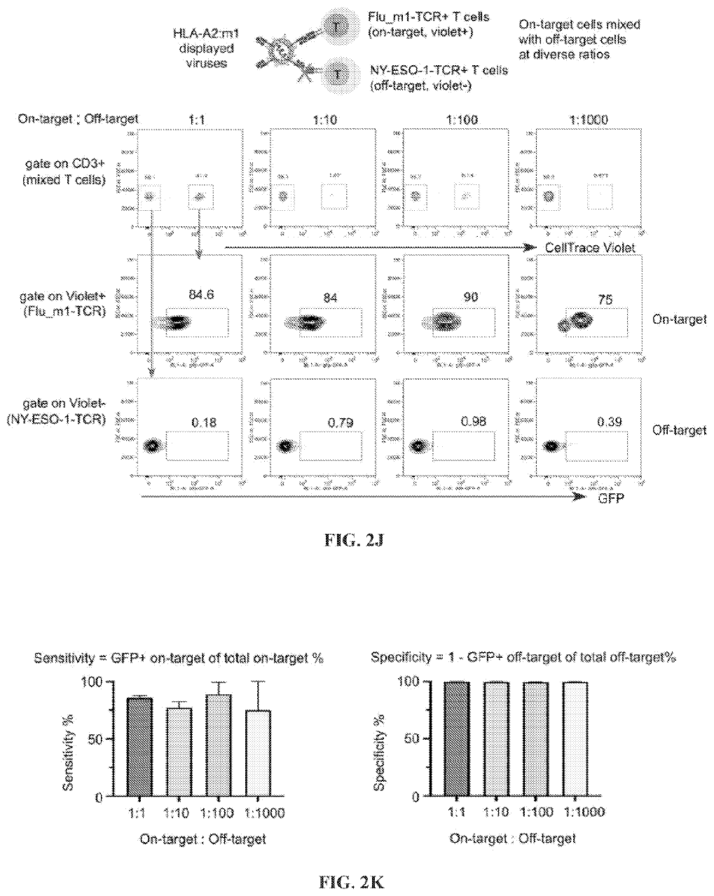

In some embodiments, the displayed pMHC can be engineered as a single chain format is used (FIG. 2A), i.e. the RNA can encode a signal peptide, an antigen peptide, a G4S linker, b2m gene, a G4S linker and MH allele in tandem.

In some embodiments, the method can further comprise the step of single-cell sequencing of the viral RNA and cell's receptor sequence to identify the MHC peptide sequence and TCR receptor information.

Other than using known pools of pMHC to identify novel TCR receptors, the methods that are provided herein can be used to identify matching MHC peptides for known T-cell receptors. For example, when the alpha and beta chains of known T-cell receptors are expressed on TCR negative cell lines such as Jurkat −76, a pool of virus with pMHCs candidates can be mixed with cells, as in described in the examples below.

### Methods for Identifying a B-Cell Receptor and Paired Antigen

Also provided herein are methods for identifying a B-cell receptor or antibody, the method comprising providing engineered lentivirus as disclosed herein, (ii) combining the lentivirus with the population of B cells; and (iii) sorting the population of cells based on the presence of the reporter thereby identifying the B-cell antigen. In some embodiments, the engineered lentivirus comprises an epitope displayed on lentivirus surface wherein the epitope is operably linked, e.g., fused with an ICAM1 transmembrane domain, a fusogen comprising a modified VSV-G viral envelope protein wherein the fusogen is capable of fusing the lentivirus to a host cell (e.g., capable of facilitating, catalyzing, or triggering fusion of the lentivirus with the host cell), wherein the host cell comprises a B-cell receptor for the intracellular epitope, a reporter transgene operably linked, e.g., fused to a lentivirus structural protein, and a barcoded RNA. In some embodiments, the antigen are cell surface membrane protein, intracellular protein, secreted protein, or other forms (e.g. glycosylated molecules) that can be expressed on cell surface.

In some embodiments, the method can further comprise the step of single-cell sequencing of the viral RNA and cell's receptor sequence to identify the antigen and matching B-cell receptor (BCR, antibody) information. In further aspects, the method can identify novel antibody/BCR and antigen pairs.

Methods for Delivering a Molecule of Interest into a Cell

In some embodiments, the present disclosure further provides a method of delivering a molecule of interest, e.g., a nucleic acid or a protein of interest, to a user-defined target cell, comprising providing an engineered lentivirus comprising a heterologous ligand displayed on a surface of the lentivirus, a fusogen comprising a modified viral envelope protein, wherein the fusogen is capable of fusing the lentivirus to a host cell (e.g., capable of facilitating, catalyzing, or triggering fusion of the lentivirus with the host cell), wherein the host cell comprises an endogenous receptor for the ligand; a reporter protein operably linked, e.g., fused to a lentiviral structural protein; and a barcoded RNA, contacting the lentivirus with a cell mixture comprising the target cell, and delivering the nucleic acid or protein only to the target cell, wherein the target cell expresses a receptor specific to the ligand on the lentivirus surface. In some embodiments, the ligand is modified in order to deliver cargo to the user-defined target cell. In some embodiments, the nucleic acid of interest is packaged inside the engineered lentiviral particle. In some embodiments, the protein of interest is operably linked, e.g., fused with a gag protein of the lentivirus. In some embodiments, the protein of interest replaces the reporter. In some embodiments, the target cell is in vivo. In some embodiments, the target cell is ex vivo. In some embodiments, the target cell is in vitro. In some embodiments, the target cell is a mammalian cell. In some embodiments, the mammalian cell is a human cell. In some embodiments, the target cell is an immune cell. In some embodiments, the immune cell is a T cell. In some embodiments, the immune cell is a T cell and the receptor is a T-cell receptor. In some embodiments, the immune cell is a B cell. In some embodiments, the immune cell is a B cell and the receptor is a B-cell receptor. In some embodiments, the human cell is a primary human blood cell (PBMC).

Methods for Delivering a Molecule of Interest into a Cell

As outlined above, one aspect of the present disclosure relates to a method for selectively depleting or enriching a target cell population in a cell mixture, the method including: providing (a) an engineered lentivirus according to any one of claims 1-17, and (b) a cell mixture comprising (i) a target cell population expressing a receptor specific for the ligand displayed on the surface of the engineered lentivirus, and (ii) a non-target cell population that does not express the receptor; contacting the engineered lentivirus with the cell population, and delivering the nucleic acid or protein only to the target cell; adding an reagent that specifically inhibits growth of the target cell population or inhibits growth of the non-target cell population, thereby selectively depleting or enriching the target cell population. In some embodiments, the receptor is an immune receptor. In some embodiments, the immune receptor is a B-cell receptor. In some embodiments, immune receptor is a T-cell receptor.

Non-limiting exemplary embodiments of the methods for selectively depleting or enriching a target cell population of the disclosure can include one or more of the following features. In some embodiments, the target cell expresses a herpes simplex virus thymidine kinase (HSV-TK) transgene, and the added reagent comprises or is ganciclovir (GCV). In some embodiments, the target cell population comprises immune cells. In some embodiments, the target cell expresses shRNA to decrease expression of cell death receptor FAS to prevent cell death of the target population. In some embodiments, the immune cells comprise a T cell. In some embodiments, the immune cells comprise a B cell.

In some embodiments, the immune cells are autoreactive immune cells. In some embodiments, the immune cells are specific for an antigen associated with a health condition. In some embodiments, the health condition is a proliferative disorder, inflammatory disorder, autoimmune disorder, or a microbial infection. In some embodiments, the proliferative disorder is a cancer. In some embodiments, the microbial infection is a bacterial infection, viral infection, or microfungal infection.

### V. Systems

Also provided herein are systems, which can be referred to as ENTER (lentiviral-mediated cell entry by engineered receptor-ligand interaction), that has been demonstrated to be a versatile platform to achieve diverse applications, for example, for ligand display, cargo delivery, and interaction recording. Exemplary applications of ENTER include decoding ligand-receptor interactions, linking receptor interaction with cell state at the single-cell level, and deliver cargos in a receptor-specific manner. As described in greater detail herein, the ability to unify multiple functionalities into one platform is a major advantage. Rather than collecting and mastering multiple distinct single-purpose technologies, ENTER offers the user one platform that solves many important problems. Firstly, in some embodiments of the disclosure, lentivirus was engineered to enable the display of heterologous cell surface proteins, intracellular and extracellular epitopes, including pMHC complexes, antibodies, co-stimulatory molecules and B cell antigens. ENTER has several advantages compared to yeast or phage display platforms (see, e.g., Table 1). The glycosylation pattern in yeast/phage display platform is different from mammalian system, which may interfere with the correct MHC presentation and recognition of paired TCRs. Moreover, yeast or phage display requires substantial optimization to achieve proper folding, stability, and presentation of MHC. Furthermore, ENTER is built in human cells and enables human glycosylation and protein folding patterns, as evidenced by Applicants' ability to present multiple HLA-peptide combinations. In addition, soluble recombinant TCR is required for screening in yeast and phage display platforms, thus making it challenging to test diverse TCRs in parallel. In contrast, ENTER allows investigators to screen primary T cell samples, opening the door to examine the vast diversity of human TCR repertoire (see, e.g., Table 1). ENTER also has advantages over cytolytic T cell reporter assay such as T-scan for decoding pMHC-TCR interaction because the latter cannot record the pairing of pMHC vs. TCR at single cell level.

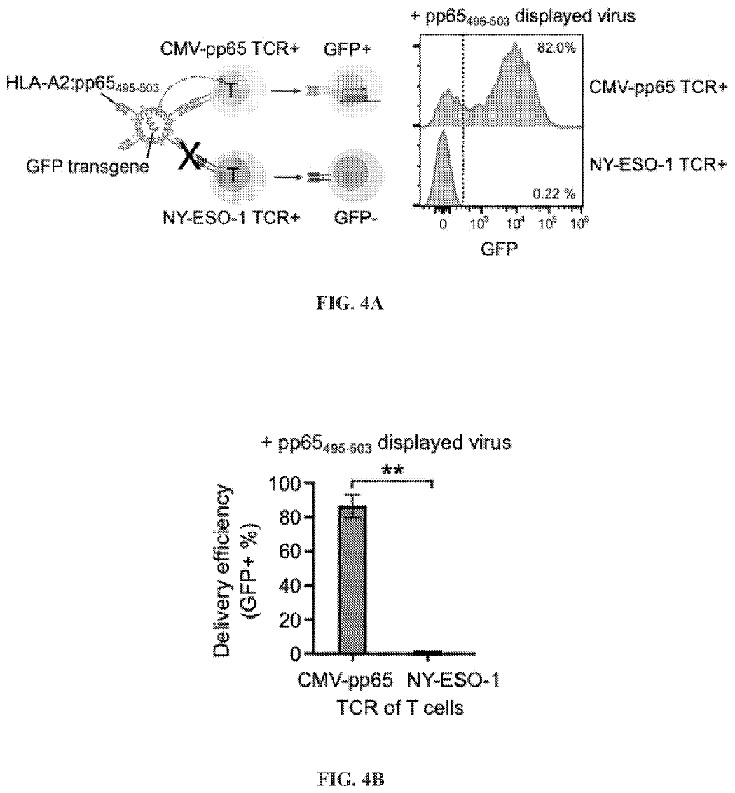

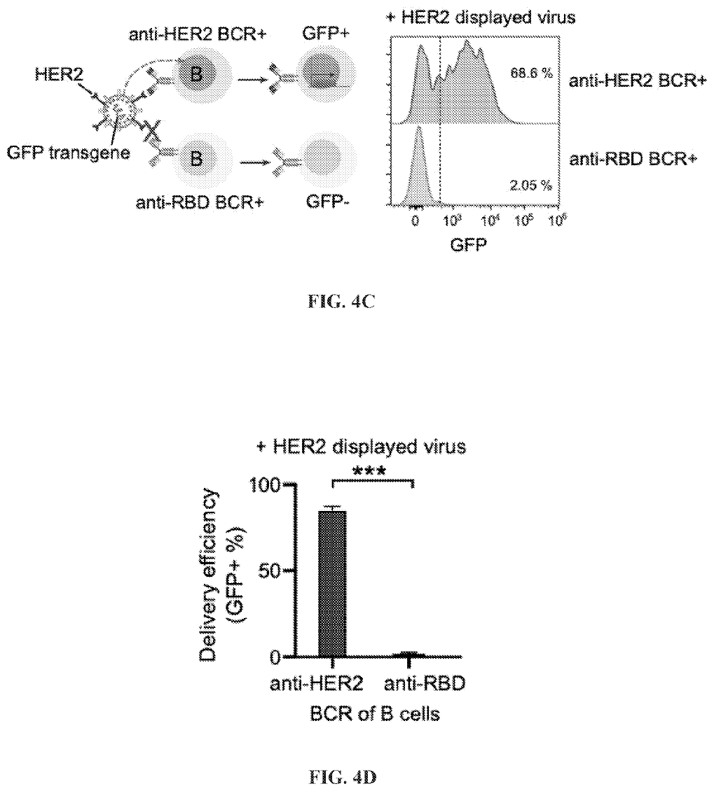

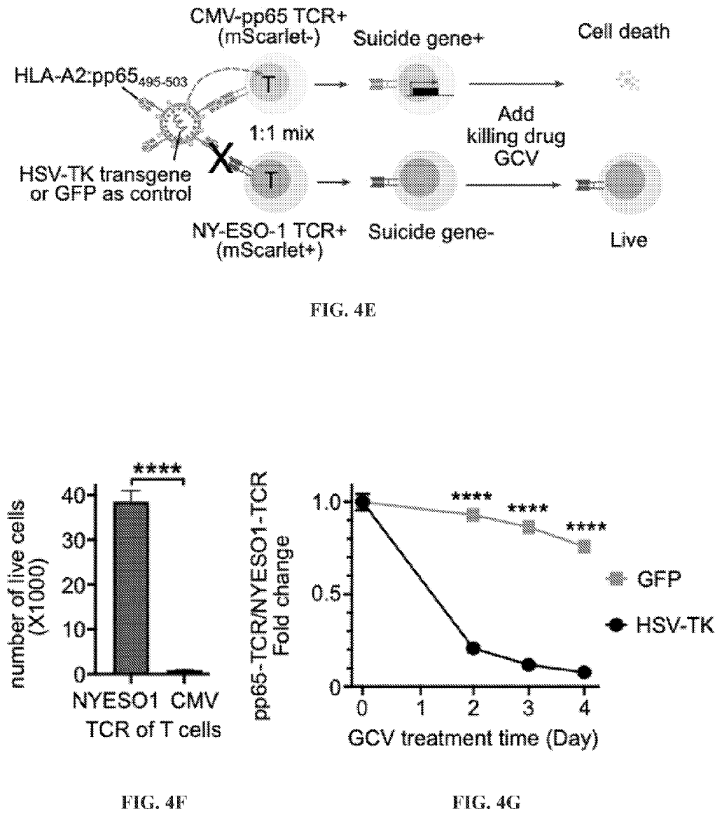

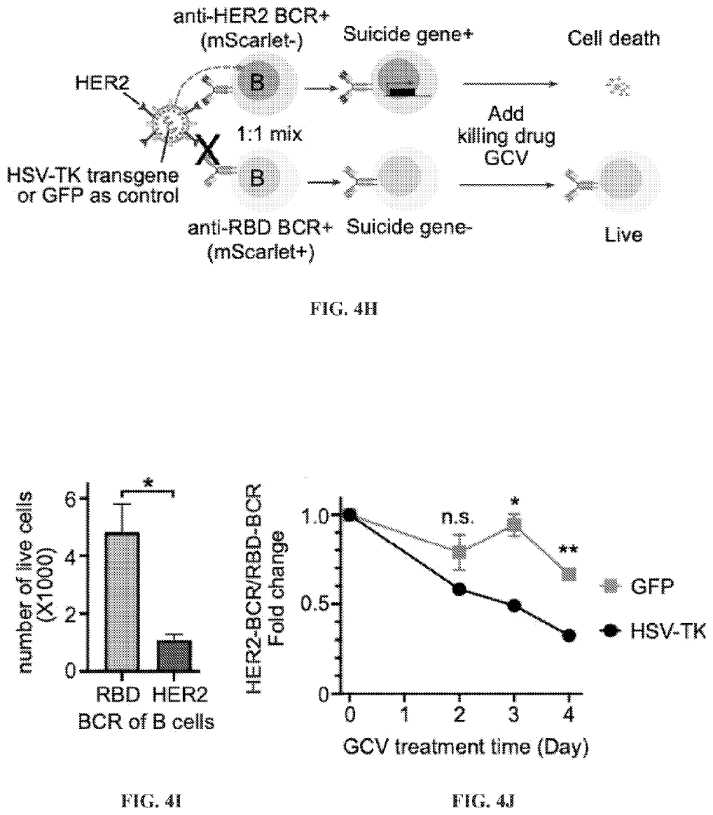

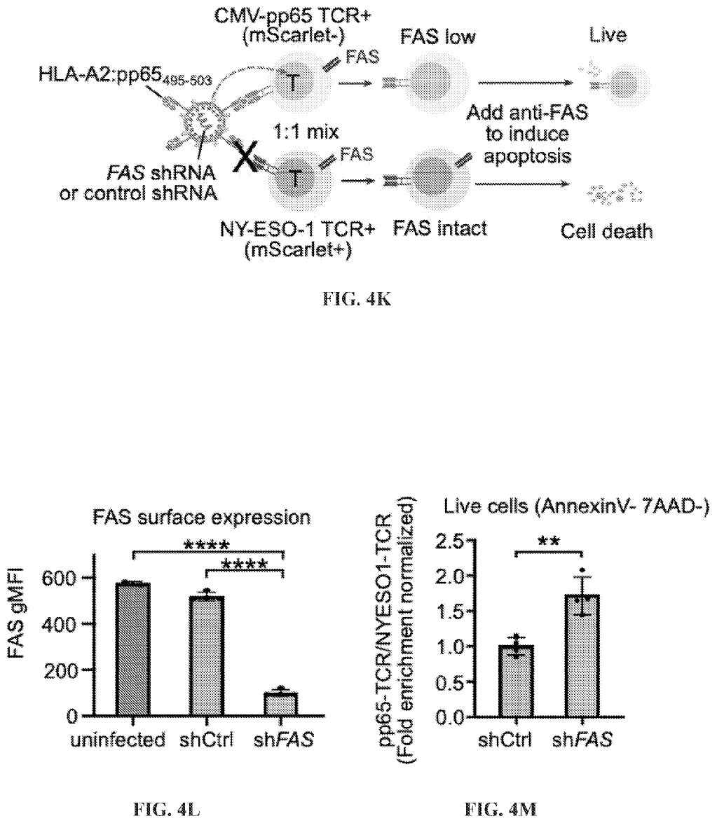

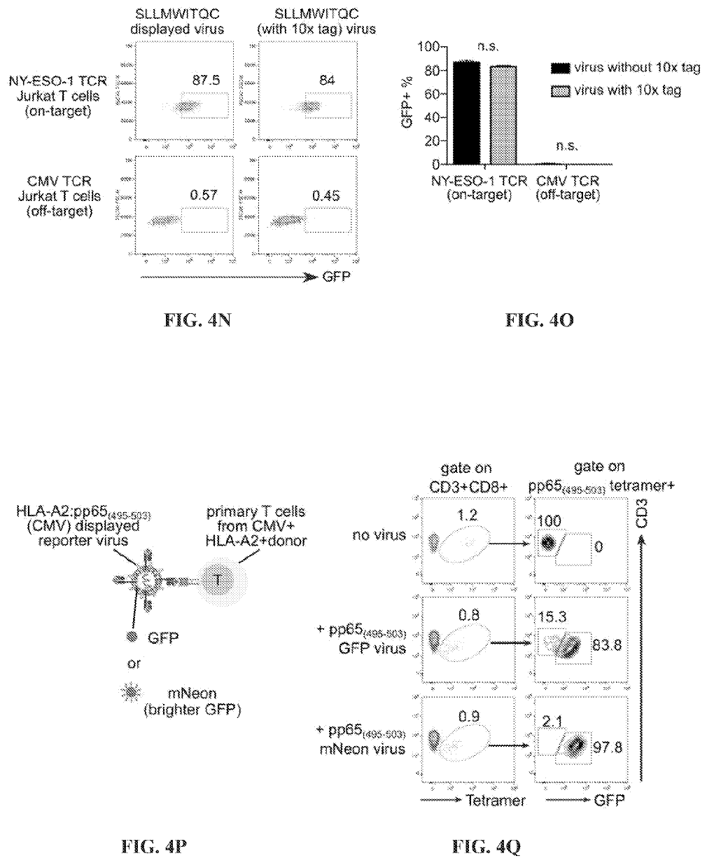

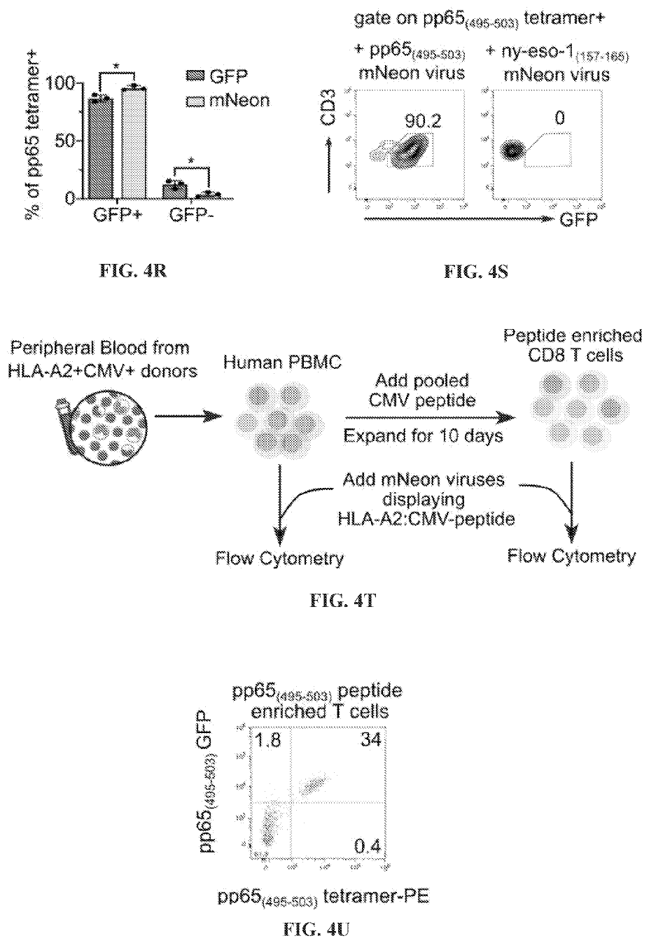

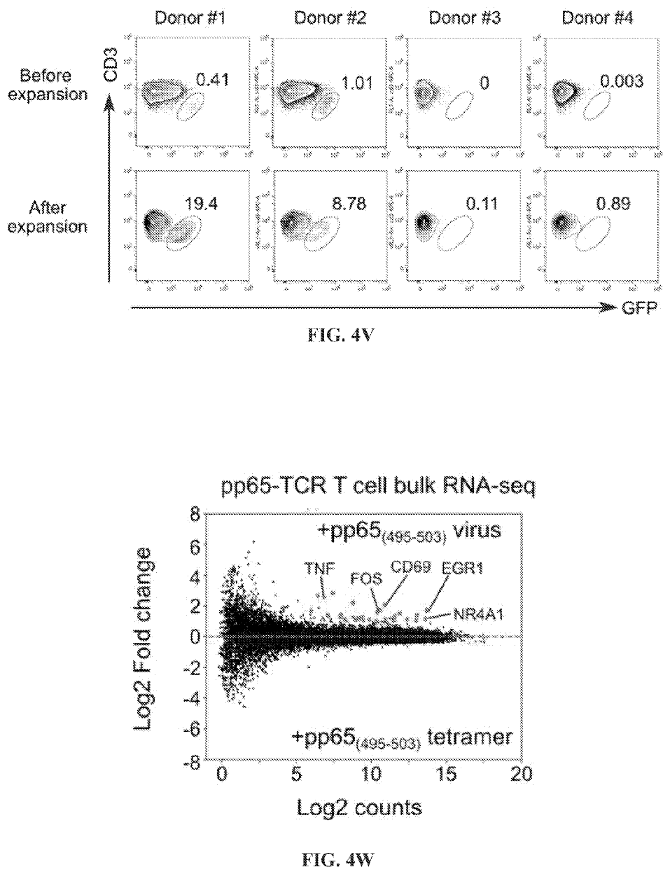

Secondly, in some embodiments, ENTER can be engineered to deliver cargos in a receptor-specific manner. The lentivirus was engineered such that receptor-ligand interaction drives viral fusion and infection. The system was further engineered so that investigators may choose to transiently or stably deliver cargos with the flexibility of using integration-defective machinery. In some embodiments, ENTER may have applications in gene therapy or RNA medicine as ENTER can achieve exquisite cell type specificity compared to existing modalities like AAV. In some embodiments, ENTER can selectively deplete or expand antigen-specific T cells, based on specific delivery of gene cargos that induce or protect from cell death (see, e.g., FIG. 4). ENTER enabled the depletion of antigen-specific B cells, which can be applied to eradicate pathogenic autoantigen-specific B cells to potentially treat autoimmune disorders.

TABLE 1
Comparison of ENTER with other platform

| | DNA | | | | | |
|---|---|---|---|---|---|---|
| | barcoded | Cell reporter | Display | | | |
| | tetramer | screen | techniques | Engineered viral platform | | |
| | (TetTCR- | (T-scan, | (Yeast, | | v- | |
| | seqHD) | SABR etc.) | phage etc.) | RAPTR | CARMA | ENTER |
| Antigen-TCR | ✓ | ✓ | ✓ | ✓ | n.d. | ✓ |
| pairing | | | | | | |
| # of antigens | 10²-10³ | 10⁵ | 10⁸ | 10² | n.a. | 10¹-10² |
| | known | known | unknown | known | | known |
| # of TCRs | 10¹⁶ | 1-10 | 1-10 | 10⁵ | n.a. | 10¹⁶ |
| | unknown | known | known | known | | unknown |
| Gene expression | ✓ | X | X | X | X | ✓ |
| | 500 | | | | | 20,000 |
| | genes/cell | | | | | genes/cell |
| Apply to primary | ✓ | ✓ | X | n.a. | ✓ | ✓ |
| sample | | | | | | |
| Antigen-specific | X | X | X | X | ✓ | ✓ |
| cargo delivery | | | | | T cell | T cell and B |
| | | | | | | cell |
| Cell type- | X | X | X | X | X | ✓ |
| specific cargo | | | | | | |
| delivery | | | | | | |

n.d.: not done
n.a.: not available

In some embodiments, ENTER as described herein can be used for linking ligand-receptor interaction with molecular blueprints at the single-cell level. For example, ENTER-seq combines the ability to decode ligand-receptor interactions with the power of single-cell genomics to resolve cell-cell communication and cell states at a massively parallel scale.

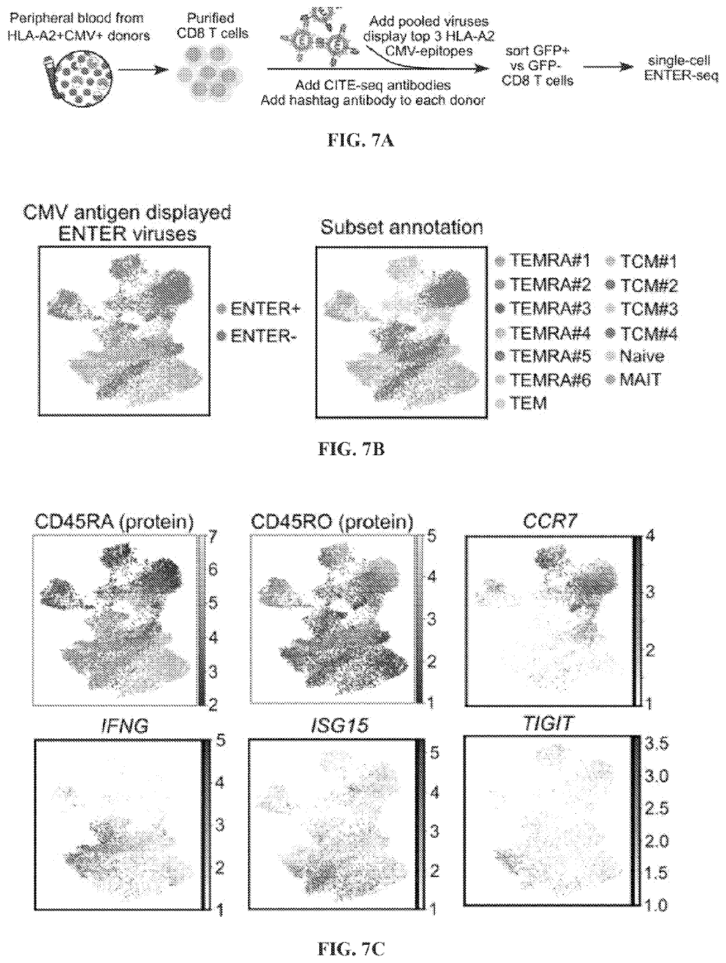

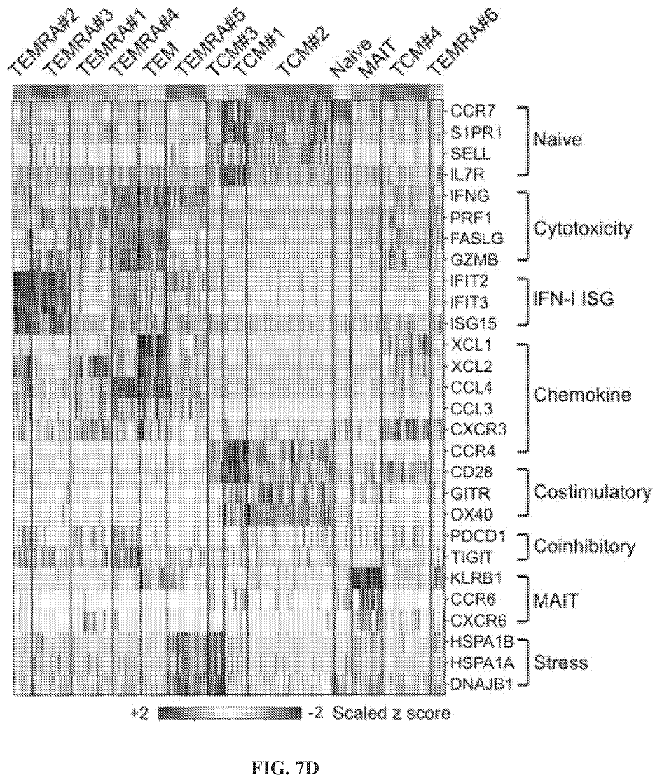

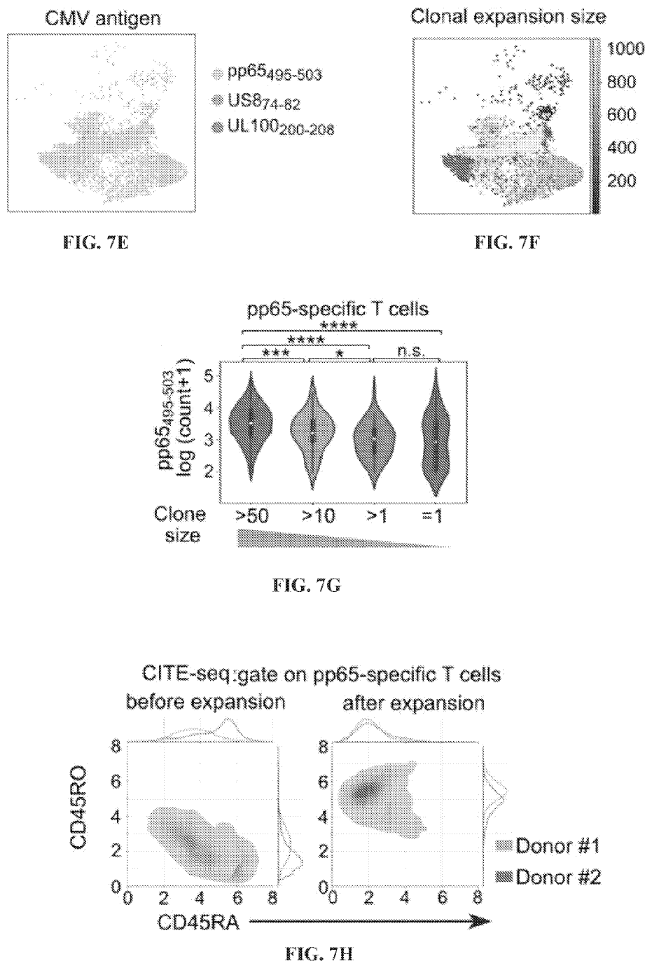

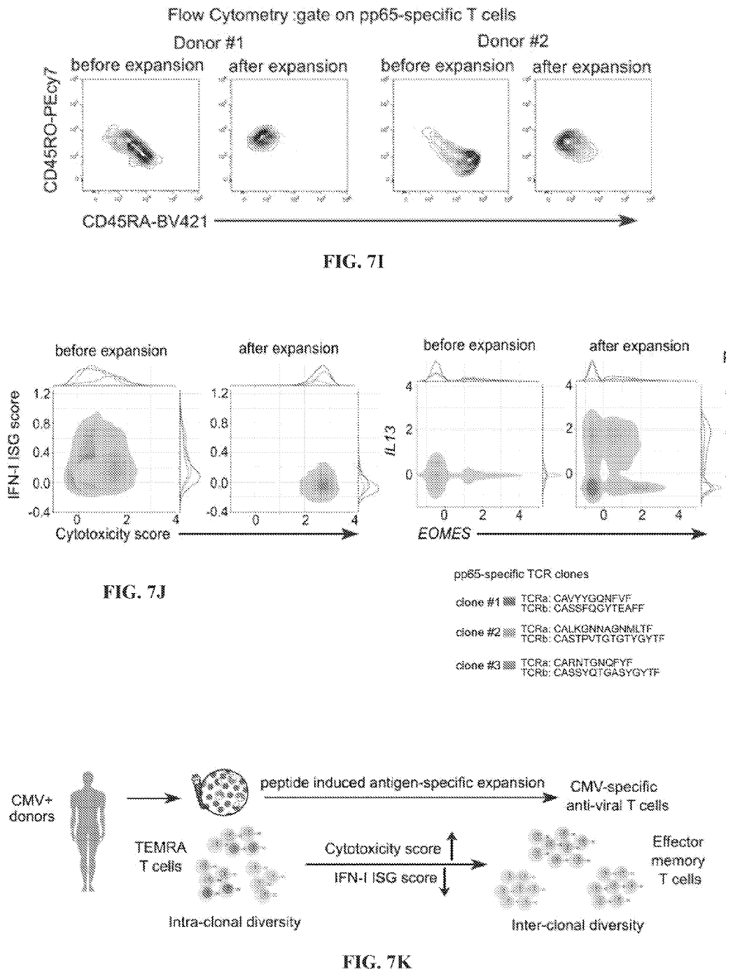

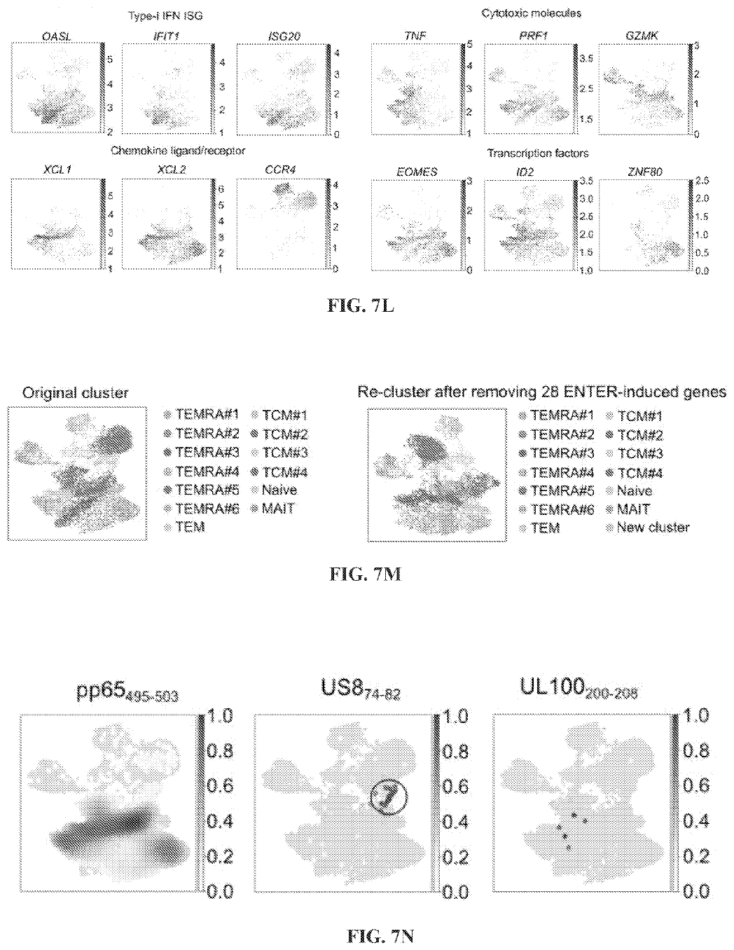

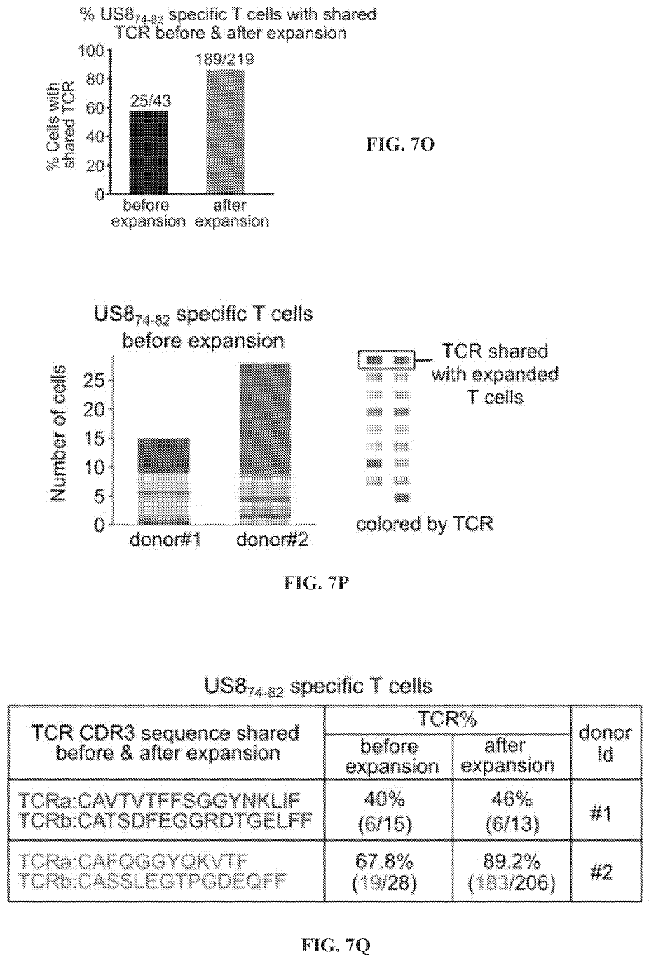

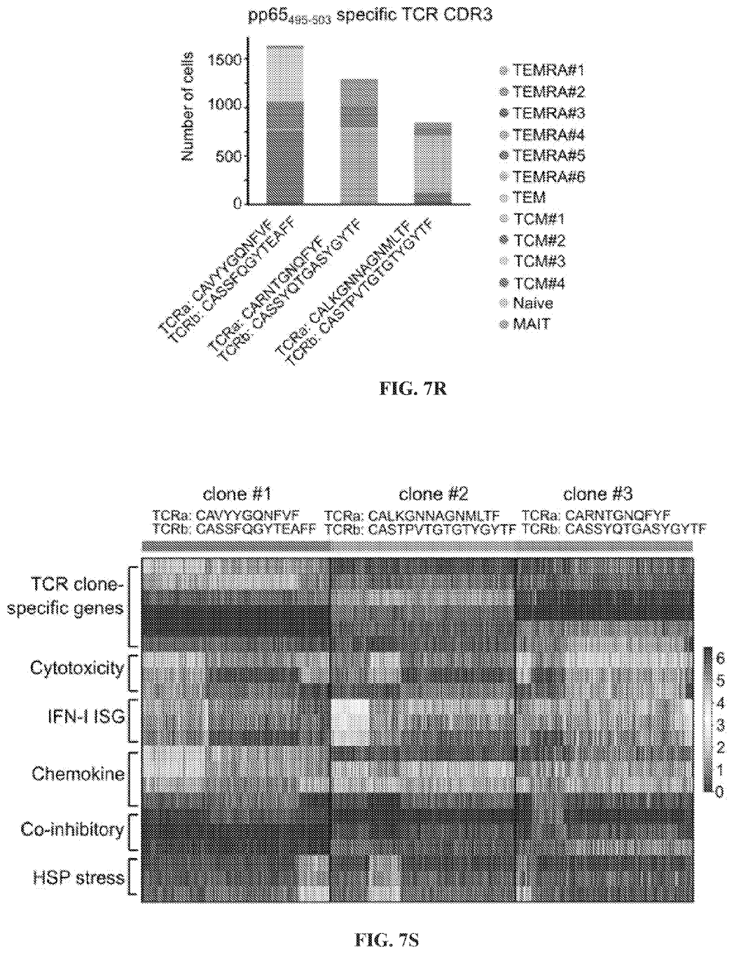

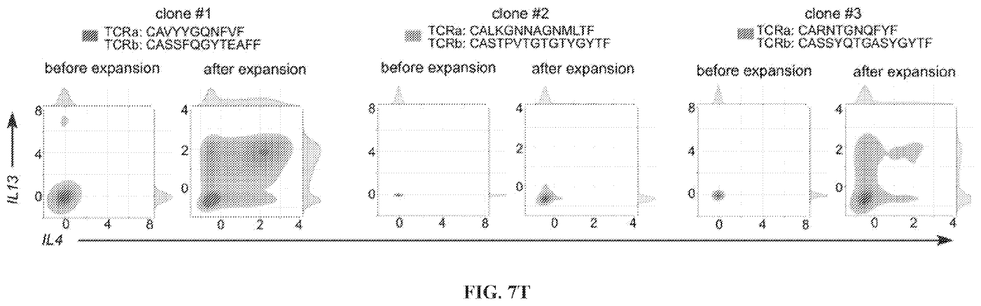

ENTER-seq for pMHC is conceptually similar to a DNA-barcoded library of pMHC tetramer molecules but with several potential advantages. Moreover, ENTER can be lower cost compared to commercial DNA barcoded pMHC tetramers. ENTER can be easily implemented in any laboratories compared to in house generation of pMHC tetramers with DNA barcodes (see, e.g., Table 2). DNA conjugation to pMHC tetramers may suffer from unequal barcode oligonucleotide loading during the conjugation reaction, whereas ENTER-seq library leverages lentiviral biology that ensures 2 copies of barcoded viral RNA for each viral particle. The uniform distribution of DNA barcode per virus like particle enabled ENTER-seq to quantify pMHC binding strength, which has not been investigated in studies using DNA barcoded pMHC tetramers. The experimental data described herein revealed that highly expanded TCR clones are associated with higher pMHC binding (see, e.g., FIG. 7G), likely resulted from higher TCR affinity to the pMHC or higher TCR surface density in expanded clones.

TABLE 2
Comparison of ENTER pMHC virus and pMHC tetramer

| | ENTER pMHC virus | DNA barcoded pMHC tetramer | |
|---|---|---|---|
| MHC | Mammalian cell line: natural MHC | E. coli bacteria system: eukaryotic MHC | |
| expression | glycosylation and proper folding in | post-translational modification and folding | |
| system | human cells | not possible | |
| Sensitivity | More sensitive than pMHC tetramer on a | Sensitive under a high concentration | |
| | molar basis per reagent | | |
| Throughput | 12 pMHC ENTER viruses for single cell | 1000 pMHC tetramers for only antigen | |
| | multi-omics profiling of antigen | specificity in bulk samples (Bentzen et al., | |
| | specificity, TCR clonality, and global | 2016). | |
| | transcriptomics. | 100-250 pMHC tetramers for single cell | |
| | | multi-omics profiling of antigen specificity, | |
| | | TCR clonality, and targeted gene | |
| | | expression(Ma et al., 2021). | |
| Barcode | 2 copy of barcoded viral RNA naturally | Individual DNA barcode conjugation | |
| | assembled per viral particle. | reaction may lead to uneven barcodes | |
| | ENTER-seq infer TCR affinity by | conjugated for tetramers. | |
| | analysis of pMHC binding per cell | No analysis of pMHC binding per cell has | |
| | | been done using DNA barcoded pMHC | |
| | | tetramers. | |
| Cost | $10 for 10 tests (directly apply to 10x | $1400 for 50 tests of commercial pMHC | |
| | Genomics platform) | tetramer (not DNA barcoded) | |
| | | >$2000 for 50 tests of DNA barcoded | |
| | | pMHC tetramer/multimer | |
| Procedure | 1. One-step Gibson assembly cloning to | Option 1 (Purchase and mix reagents in a | |
| | insert DNA oligo sequence (encoding 9aa | convenient but high-cost manner): | |
| | peptide) into pMHC plasmid vector. | Purchase peptides (GenScript), DNA | |
| | 2. Transfection of pMHC vector along | barcoded and fluorophore conjugated | |
| | with other ENTER vectors in HEK293T | Klickmer (Immudex), and MHC-I | |
| | cells. | monomers (immuneAware) (Total >$2000 | |
| | 3. Collect supernatant containing viruses | for 50 tests). Mix peptides to MHC-I and | |
| | after 48 h and 72 h after transfection. | Klickmer at specific ratios individually. | |
| | | Option 2 (Generate most reagents in house | |
| | 4. Viral concentration: add 1 mL Lenti-X | in a relatively low-cost but more | |
| | concentrator per 2 mL supernatant, mix | complicated manner) | |
| | well and incubate at 4° C. overnight, | 1. Generation of biotinylated MHC-I | |
| | 2000Xg spin down for 45 min, resuspend | monomers: | |
| | pellet in 150 uL DMEM medium (20X | (1) | Expression and purification of |
| | concentration) | | MHC-I light chain B2M and heavy |
| | | | chain using bacteria system (25 |
| | | | steps). |
| | | (2) | MHC in vitro refolding, |
| | | | biotinylation and purification (25 |
| | | | steps) |

| 2. Generation of peptides by in vitro | |
|---|---|
| transcription and translation. | |
| 3. Generation of DNA barcoded fluorescent | |
| streptavidin. | |
| (1) | Conjugation of DNA linker to |
| | commercial fluorescent labeled |
| | streptavidin ($500). |
| (2) | Annealing and overlap extension to |
| | conjugate DNA barcodes to |
| | complementary DNA linker |

| | | |
|---|---|---|
| | | 4. pMHC UV exchange of peptides, |
| | | tetramerization, and purification (8-10 |
| | | steps). |
| Specialized | None | Size exclusion chromatography (HPLC) |
| equipment | | system with a gel-filtration column is |
| | | required for purification of pMHC |
| | | monomers. |

Finally, as described in greater detail herein. ENTER is more sensitive than pMHC tetramer on a molar basis per reagent (see, e.g., FIG. 2C). In some embodiments, the superior sensitivity may arise from a high number of pMHCs that are displayed on ENTER. HIV-based lentiviral particle displays 14-100 molecules of envelope protein per viral particle whereas pMHC tetramers are 4 linked molecules by definition. Together, ENTER-seq allows investigators to record ligand-receptor specificity and read out the biological consequences of this interaction, such as antigen-dependent T cell fates including naïve cell activation, effector cell expansion, memory cell formation, or T cell exhaustion. Similarly, ENTER-seq may be used to understand the molecular programs of antigen-specific B cells in the context of infectious diseases and autoimmunity.

ENTER-seq analysis of primary CMV-specific T cells demonstrates the power of the platform to connect the landscape of antigen epitope, TCR repertoire, gene expression program, and surface protein phenotypes across tens of thousands of primary T cells in a single experiment. Such massively parallel profiling of diverse modalities uncovered donor-specific antigen specificity and immunogenicity of viral epitopes. In some embodiments, ENTER-seq of T cells pre- and post-peptide stimulation unveiled transcriptional alteration upon expansion and inter-clonal phenotypic diversity in response to the same antigen. Such transcriptional changes and clonal divergence in Th2 cytokine expression might be impacted by the different TCR affinity/avidity/density to the same pMHC antigen, or different priming environment from antigen-presenting cells. A recent study of single cell profiling CD19-CAR T cells in acute lymphoblastic leukemia patients showed that an induction of Th2 expression is positively associated with clinical efficacy in durable responders compared to relapsed patients (Bai et al., 2022). However, it is unclear how Th2 cytokines can boost CD8 T cell effector function to achieve long-term remission and if such benefit can be generalizable to infectious disease. The experimental data described herein showing TCR clone-specific induction of Th2 cytokine expression might inform the selection of TCR to engineer TCR-T cells for adoptive T cell therapy. Furthermore, ENTER-seq as described herein provides insights into a comprehensive understanding of how T cell clonality and specificity influence the molecular phenotypes and physiological function of antigen-specific T cells.

In some embodiments, ENTER as described herein may be used to isolate and enrich tumor antigen-reactive T cells to infuse back into patients. In some embodiments, ENTER as described herein may be further used in a discovery context to screen immunogenic antigen or elite TCRs for the rational design of vaccine development or cancer immunotherapy. In some embodiments, ENTER as described herein may also be applied to screen BCRs that target viral antigens, facilitating the development of therapeutic antibodies to prevent viral infections. In some embodiments, ENTER as described herein enables antigen-specific delivery of cargos such as genes and shRNAs, allowing perturbation and manipulation of antigen-specific T and B cells. This targeted delivery strategy might be applied to reinvigorate exhausted anti-tumor T cells without triggering the immune-related adverse events, or deplete autoreactive T or B cells to treat autoimmunity. In yet further embodiments, ENTER may be extended to additional receptor-ligand pairs, such as G-protein coupled receptors, adhesion molecules, or protocadherins. Therefore, ENTER may be used to address cell-cell connectivity beyond the immune system.

ENTER-seq can combine the ability to decode ligand-receptor interactions with the power of single-cell genomics to resolve cell type and cell states at a massively parallel scale. ENTER-seq for pMHC is conceptually similar to a DNA-barcoded library of MHC tetramer molecules but with several potential advantages. MHC tetramer libraries require individual peptide synthesis and then loading into MHC tetramers, leading to high cost, long lead time, and lower throughput compared to ENTER-seq library that can be prepared by massively parallel DNA synthesis. DNA conjugation to MHC tetramers may suffer from unequal barcode oligonucleotide loading during the conjugation reaction, whereas ENTER-seq library leverages lentiviral biology that ensures 2 copies of barcoded viral RNA for each viral particle. Finally, ENTER-seq may be more sensitive compared to MHC tetramers. HIV-based lentiviral particle displays 14-100 molecules of Env protein per viral particle whereas MHC tetramers are four linked molecules by definition.

ENTER-seq allows investigators to record ligand-receptor specificity and readout the biological consequences of this interaction, such as antigen-dependent T cell fates such as naïve cell activation, effector cell expansion, memory cell formation, or T cell exhaustion. Similarly, ENTER-seq may be used to understand the molecular programs of autoantibody producing B cells in autoimmunity.

ENTER links ligand-receptor interaction with molecular blueprints at single-cell resolution. ENTER has advantages over cytolytic T cell reporter assay such as T-scan because the latter cannot record the pairing of peptide-MHC vs. TCR at single cell level, which precludes pooled analyses.

ENTER may have translational application in immunology and beyond. ENTER may be used to isolate and enrich tumor antigen-reactive T cells to infuse back into patients. The non-integrative nature of ENTER facilitates adoptive T cell therapy. ENTER may be furthered used in a discovery context to screen immunogenic antigen or elite TCRs for rational design of vaccine development or cancer immunotherapy.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

The discussion of the general methods given herein is intended for illustrative purposes only. Other alternative methods and alternatives will be apparent to those of skill in the art upon review of this disclosure, and are to be included within the spirit and purview of this application.

Although the present disclosure has been described in some detail by way of illustration and example for purposes of clarity and understanding, it will be apparent that certain changes and modifications can be practiced within the scope of the appended claims.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### EXAMPLES

### Example 1

This Example describes the results of experiments performed to illustrate an exemplary ENTER in accordance with some embodiments of the disclosure where ENTER is engineered to be a modular viral display and delivery platform to capture and decode ligand-receptor interactions, deliver cargos in target cells, and connect ligand-receptor interactions with cellular state.

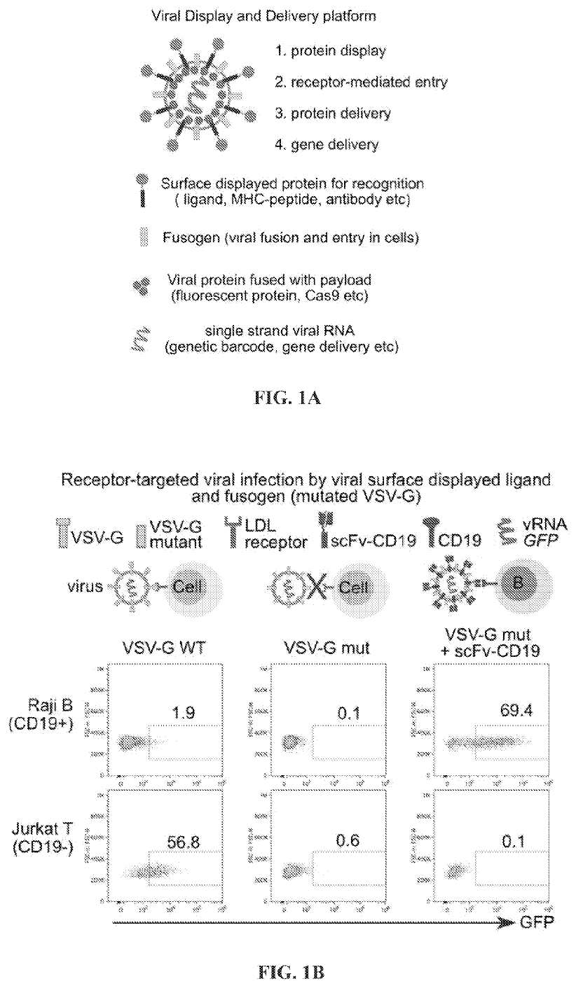

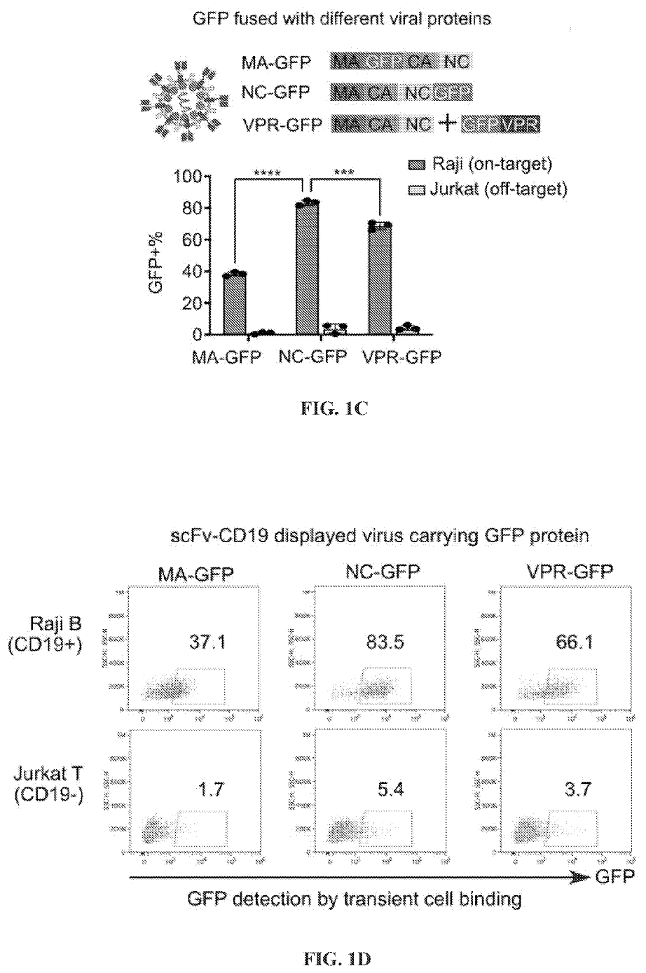

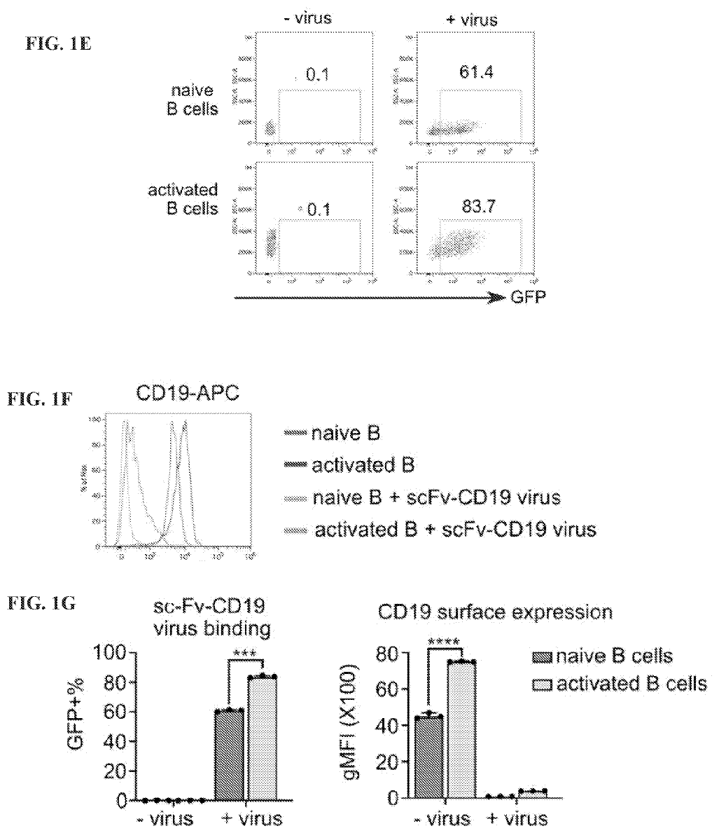

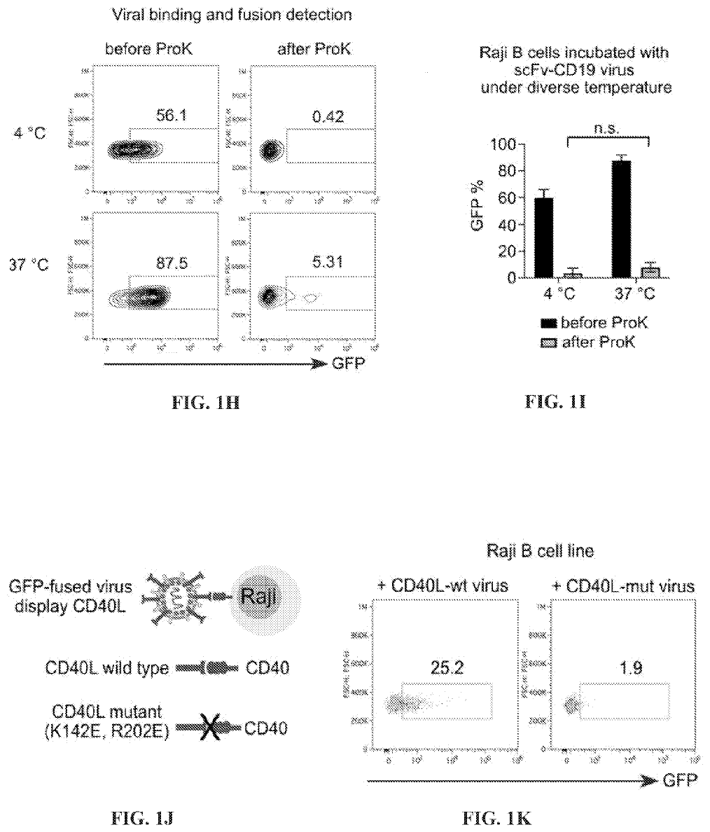

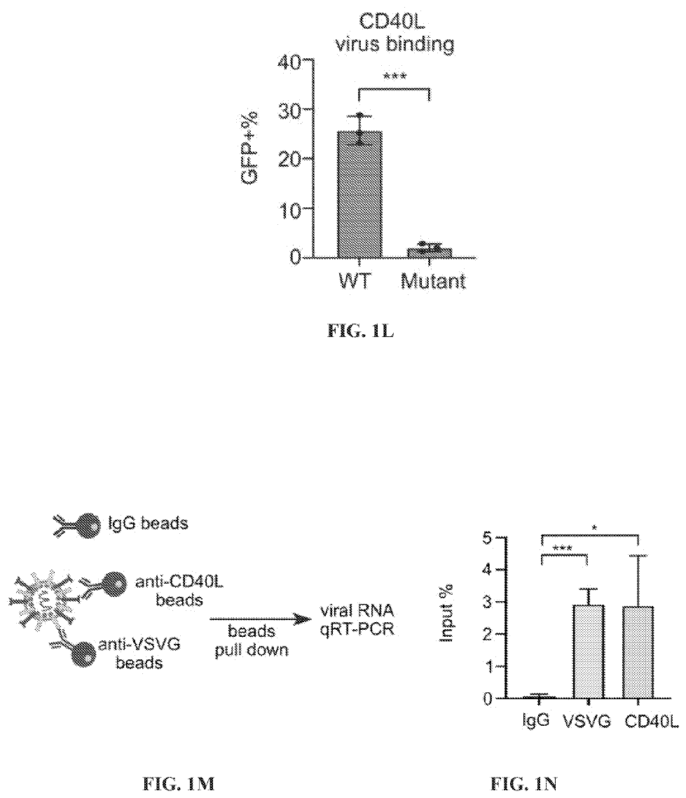

Lentivirus was engineered at multiple levels including (i) ligand proteins displayed on viral surface, (ii) host receptor-targeted viral entry by displayed ligand and modified fusogen, (iii) fluorescent protein delivery via fusion of viral capsid, and (iv) tagged viral RNA for single cell sequencing (see, e.g., FIG. 1A).

To achieve specific ligand-receptor interaction between lentiviruses and host cells, for example, by using viruses displaying user-defined ligand proteins, a viral envelope protein with disrupted native receptor binding while maintaining intact fusion ability (termed as fusogen) is designed to cooperate with user-defined ligand proteins displayed on viral surface. The cooperation of two separate modules (ligand protein+fusogen) allows the interaction between viral-displayed ligand and host receptor to further facilitate viral fusion to host cells by fusogen (see, e.g., FIG. 1A). The vesicular stomatitis virus G protein (VSV-G), a viral envelope protein is used to pseudotype lentiviruses. VSV-G pseudotyped viruses have a broad tropism since VSV-G can recognize and interact with Low Density Lipoprotein Receptor (LDLR), which is expressed in many cell types.

In these experiments, Jurkat T cells and Raji B cells were infected with the VSV-G pseudotyped lentiviruses carrying GFP transgene. A robust GFP expression in these Jurkat T cells and Raji B cells was observed, although with different transduction efficiency which is potentially due to variable expression of LDLR on diverse cell types (see, e.g., FIG. 1B).

A VSV-G mutant was engineered which harbors two point-mutations (K47Q, R354A) to prevent its recognition and interaction with LDLR on host cells (Nikolic et al., 2018) A minimal GFP expression in Raji (0.1%) and Jurkat (0.6%) cells using mutant VSV-G pseudotyped viruses was observed (see, e.g., FIG. 1B), suggesting that the viral recognition of specific receptors on host cells is the first essential step for viral entry and integration. To test if the VSV-G mutant is a good fusogen candidate to cooperate with user-defined ligands on the viral surface for viral infection in host cells expressing paired receptors, a well-established CD19-CAR (chimeric antigen receptor) that contains an anti-CD19 single-chain antibody variable fragment (sc-Fv), was co-expressed with the VSV-G mutant, the GFP transgene in a viral transfer vector, and packaging component for lentiviruses in HEK293 T cells and viruses from supernatant were collected. As expected, such viruses specifically infected Raji B cells with high expression levels of CD19 but not CD19-negative Jurkat T cells (see, e.g., FIG. 1B). Thus, the Applicants have developed a viral display platform that can reprogram the viral fusion from cell entry that is dependent on the native VSV-G/LDLR interaction to cell entry that is dependent on the interaction between user-defined ligand and paired host cell receptors.

In order to capture ligand-receptor interaction while avoiding the viral integration and integration-induced mutagenesis of host genome (Ranzani et al., 2013), Applicants engineered a viral integrase mutant (D64V) that cannot integrate host genome (Certo et al., 2011) and fused the GFP protein with viral structural proteins to track ligand displayed viruses. The transient viral entry into host cells expressing paired receptors using ligand-displayed viruses carrying GFP protein instead of viral integration to express GFP was measured. To identify the viral protein that serve as an optimal fusion partner for GFP, Applicants tested three viral proteins including matrix protein (MA), nucleocapsid protein (NC), and HIV accessory protein named viral protein R (VPR) (see, e.g., FIG. 1C). During viral assembly and synthesis, MA and NC are processed from Gag precursor protein which can be assembled in cis as 3000 copies of MA or NC per viral particle ((De Guzman et al., 1998; Kutluay et al., 2014) VPR can be incorporated in trans into the viral particle via interaction with Gag protein as 500 copies of VPR per viral particle (Wu et al., 1995). It was observed that 80% of Raji cells bound by CD19-CAR presented viruses when GFP was fused with NC, which significantly outperforms MA-GFP and VPR-GFP (see, e.g., FIG. 1C).

To investigate if the NC-GFP viruses displaying CD19-CAR can recognize and bind to primary CD19+ B cells from human blood, these viruses were incubated with naïve or activated human primary B cells for 2 hour and detected the GFP signal on these B cells by flow cytometry. Similar to Raji B cell line, 80% of activated human primary B cells were bound by NC-GFP labeled CD19-CAR displayed viruses whereas 60% of naïve B cells are GFP+ (see, e.g., FIG. 1D). The difference of CD19 expression between naïve and activated B cells might account for discrepancy of binding of CD19-CAR viruses. Indeed, flow cytometry results showed that the surface expression of CD19 in activated B cells was significantly higher than that of naïve B cells (see, e.g., FIGS. 1E-1F), which is consistent with the higher binding of viruses on activated B cells. This result indicates that the binding of ligand-displayed viruses to receptor-expressed cells is quantitatively correlated with the expression level of ligand-paired receptors. Moreover, CD19 surface expression is dramatically decreased after incubation of CD19-CAR viruses, indicating that CD19-CAR viruses specifically bind to CD19 to prevent the later binding of flow cytometry antibody targeting CD19, either through CD19 antigen masking or inducing internalization of surface CD19 (FIGS. 1E-1F). To determine if the loss of surface CD19 is due to internalization of surface CD19 induced by viral fusion or specific viral bind to CD19 which prevent later CD19 antibody staining, the viral binding and fusion assay were performed (see, e.g., FIG. 1H). The result showed that there were only 5% GFP+ cells after proteinase K treatment, indicating that very few cells have undergone viral fusion to prevent the proteinase K mediated degradation of surface bound GFP labeled viruses (see, e.g., FIG. 1I). Thus, the reduction of surface CD19 is mostly resulted from the specific CD19 binding/masking from ENTER viruses rather than internalization, further highlighting that the ligand-displayed viruses are highly specific to the targeted receptors.

To further determine if the ligand-displayed GFP fused viruses can specifically bind to target cells through other ligand-receptor interactions, viruses were engineered to display either wild type CD40 ligand (CD40L) or mutant CD40L. The CD40L mutant contains two point-mutations (K142E, R202E), leading to decreased binding affinity towards CD40 (Pasqual et al., 2018) The flow cytometry results showed a significant decrease of GFP+ Raji cells when incubated with viruses displaying CD40L mutant compared to wild-type CD40L (FIGS. 1J-1L).

To confirm the incorporation of ligand protein and fusogen on virion surfaces, an immunocapture assay was performed to pull down viruses that display desired proteins by antibody coated magnetic beads (see, e.g., FIG. 1M). Specifically, viruses displaying CD40L and fusogen (VSV-G mutant) were generated and then incubated them with anti-CD40L beads, anti-VSV-G beads, and IgG beads respectively. After incubation and extensive washing, the viral RNA was extracted for subsequent qRT-PCR analyses. Compared to IgG negative control, a significant enrichment of viral RNA in anti-CD40L and anti-VSV-G group was found, validating the incorporation of ligand proteins and fusogens on virion surfaces (see, e.g., FIG. 1N).

Together, these results suggest that Applicants' viral display platform, e.g., ENTER, can capture a highly specific ligand-receptor interaction in a transient viral binding assay and is applicable to multiple categories of receptor-ligand interactions.

Therefore, engineered lentiviral particles displaying specific ligands deliver fluorescent protein into target cells upon cognate receptor-ligand interaction, without genome integration or transgene transcription.

### Example 2

This Example describes the results of experiments performed to illustrate how ENTER with MHC-peptide (pMHC) displaying viruses maps TCR specificity, particularly how this viral display platform captures the interaction between pMHC and TCR.

Applicants engineered viruses to display a single chain of MHC fused with beta 2 microglobulin (B2M) and covalently linked peptide (see, e.g., FIG. 2A). To prevent interference of the endogenous human leukocyte antigen (HLA, the human MHC locus) from HEK 293T cells when producing HLA-peptide displayed viruses, Applicants made a stable HLA knockout (KO) HEK 293T cell line by knocking out all potential HLA class I alleles (HLA-A/B/C) by CRISPR-Cas9. The surface expression of B2M is also abolished in HLA KO cells, suggesting all endogenous HLA alleles have been successfully deleted (see, e.g., FIG. 2G). The single chain of HLA-A*0201 (HLA-A2) fused with B2M and peptide was overexpressed in HLA KO cells and observed a high level of surface expression of HLA-A2 and B2M (see, e.g., FIG. 2G).

Applicants engineered GFP-fused reporter viruses to display pMHC on surface by co-expressing the single-chain trimer of pMHC, mutant VSV-G, and viral Gag protein containing NC-GFP in HLA KO HEK 293T cells. The viruses were collected and incubated with Jurkat T cells expressing a TCR that targets the cognate pMHC antigen. Using these modular components, virus displaying a well-established cancer-testis antigen NY-ESO-1 as a 9-mer peptide (SLLMWITQC) on HLA-A2, a most prevalent HLA allele in humans was successfully generated (Jager et al., 1998) (see, e.g., FIG. 2A). 88.2% of cognate NY-ESO-1 TCR-expressing T cells were labeled by NY-ESO-1 antigen-bearing GFP viruses, compared to 1.26% of T cells specific to a known cytomegalovirus (CMV) epitope (Lee and Meyerson, 2021). Similarly, viruses displaying a CMV antigen as an 11-mer (YSEHPTFTSQY) peptide on a different HLA allele HLA-A*01:01, specifically entered into CMV TCR-T cells rather than NY-ESO-1 TCR-T cells (see, e.g., FIG. 2A). Applicants further engineered viruses displaying diverse 9-mer antigen epitopes from cancer-testis antigen, CMV pp65 antigen (ny-eso-1₁₅₇₋₁₆₅), CMV pp65 antigen (pp65₄₉₅₋₅₀₃), and influenza matrix protein antigen (m1₅₈₋₆₆), all of which are presented on HLA-A2 allele (Gotch et al., 1987; Wills et al., 1996). The result showed over 87% of TCR-matching T cells were GFP+ after 2 hours of incubation with GFP fused viruses displaying cognate HLA-A2-peptide whereas only 1% of these T cells were labeled by negative control antigen displayed GFP viruses (see, e.g., FIG. 2B). The highly specific entry of pMHC displaying viruses into TCR matching T cells were observed with different antigen peptide lengths and distinct HLA alleles, highlighting the generality of the ENTER platform to present diverse pMHC antigens.

To further test the specificity of the pMHC displayed viruses, the CMV pp65₄₉₅₋₅₀₃ antigen-specific TCR Jurkat T cells was first incubated with pp65₄₉₅₋₅₀₃ displayed viruses and then stained with a widely used commercial pp65₄₉₅₋₅₀₃ tetramer. For negative controls, these T cells were incubated with the influenza m1₅₈₋₆₆ displayed viruses and commercial m1₅₈₋₆₆ tetramer (see, e.g., FIG. 2H). Flow cytometry result showed that over 90% of tetramer positive cells were GFP+, indicating a strong concordance of pp65₄₉₅₋₅₀₃ tetramer staining with the binding of pp65₄₉₅₋₅₀₃ displayed GFP viruses. The negative control influenza m1₅₈₆₆ tetramers and viruses did not label pp65₄₉₅₋₅₀₃ TCR T cells (see, e.g., FIG. 2H). Additionally, it was observed that the pp65₄₉₅₋₅₀₃ tetramer intensity and CD3 surface expression were significantly decreased after co-incubation with viruses displaying pp65₄₉₅₋₅₀₃ (see, e.g., FIG. 2I), which is similar to the observation of decreased surface expression of CD19 on Raji B cells after binding of CD19-CAR displayed viruses. This result indicated that pMHC displayed viruses specifically bind to and mask the TCR-CD3 complex, preventing later binding of pMHC tetramer and anti-CD3 antibody.

After determining the specificity of pMHC displayed viruses using pMHC tetramers as references, Applicants compared the sensitivity between pMHC displayed viruses and pMHC tetramers on a molar basis per reagent (See, e.g., General Methods in Example 11). The result showed that 2×10⁸ ENTER viral particles can stain 95.7% TCR-T cells whereas 2×10⁸ pMHC tetramers cannot detect any TCR-T cells (see, e.g., FIG. 2C). Importantly, the binding efficiency of 2×10⁸ ENTER viruses is similar to 8×10⁹ pMHC tetramers, suggesting that ENTER viruses is more sensitive (˜40 fold) than pMHC tetramers.

To further determine if the TCR affinity to pMHC impact the binding of pMHC display viruses to TCR expressing T cells, a TCR-T cell line (1G4 wt) that recognizes NY-ESO-1 antigen variants with different known TCR affinities (Kd range from 7-85 μM) (Zhang et al., 2021) was generated. The NY-ESO-1 TCR T cell line utilized in previous experiments (see, e.g., FIGS. 2A-2C) is very similar to 1G4 wt TCR T cell line except a few mutations on its TCR, resulting in very high binding affinity to ny-eso-1₁₅₇₋₁₆₅ antigen (Robbins et al., 2008). ENTER was then engineered to display different ny-eso-1₁₅₇₋₁₆₅ antigen peptide variants such as wild-type peptide (SLLMWITQC), L3A mutant (SLAMWITQC), and T7A mutant (SLLMWIAQC). Then the percentage of GFP+ cells was measured after incubating different TCR-T cells with antigen variants displayed viruses after normalizing the titer of viruses (showed by viral p24 protein level). The result showed that ENTER is sensitive to detect TCR affinity as low as 10.8 uM when adding high titer of viruses (40 ng p24). For very low TCR affinity like 84.9 uM, 25% of on-target cells can still be detected by ENTER under a high titer (see, e.g., FIG. 2D), highlighting a wide spectrum of TCR affinity that ENTER can recognize. It was observed that the TCR binding affinity is positively correlated with ENTER recognition efficiency, suggesting that ENTER might be applied to infer relative TCR affinity by measuring binding efficiency of pMHC displayed viruses (see, e.g., FIG. 2D).

In addition, to determine the specificity and sensitivity of the pMHC viral display platform, Applicants mixed on-target T cells (TCR recognizing m1₅₈₋₆₆ antigen) and off-target T cells (TCR recognizing ny-eso-1₁₅₇₋₁₆₅ antigen) at different ratios and then incubated with the m1₅₈₋₆₆ antigen presented GFP viruses (see, e.g., FIGS. 2E, 2J). To distinguish these two different TCR T cell line, the flu-M1 TCR T cells were labelled with cell trace violet dye. The signal-to-noise ratio based on the frequency of on-target GFP+ cells versus that of off-target GFP+ cells was calculated. The signal-to-noise ratio was over 150-fold even when the frequency of on-target T cells is as low as 1 in 1000, demonstrating a high specificity and sensitivity of the ENTER viral display platform (see, e.g., FIGS. 2F, 2J, and 2K).

Taken together, the experimental data described above demonstrates that ENTER captures the interaction between pMHC and TCR in a specific and sensitive manner.

### Example 3

This Example describes the results of experiments performed to illustrate that an exemplary decoding of B cell specificity by ENTER viruses that display B cell antigens.

B cells possess a high diversity of BCR that can specifically target foreign antigens from invading viruses and self-antigens. Viral antigen-specific B cell can produce antibodies (secreted form of BCR), which is beneficial to prevent viral infection. In contrast, autoantigen-specific B cells can produce detrimental autoantibodies attacking ourselves, contributing to autoimmune disorders (Burbelo et al., 2021; Tan, 1989). Thus, it is important to decode B cell specificity, which will facilitate the development of highly effective anti-viral antibodies, guide the rational design of vaccines, and provide a better understanding of the formation of autoreactive B cells (Ju et al., 2020). Based on the successful application of ENTER in decoding T cell specificity, this Example describes the results of experiments performed to explore the feasibility of capturing the interaction between BCR and antigens.

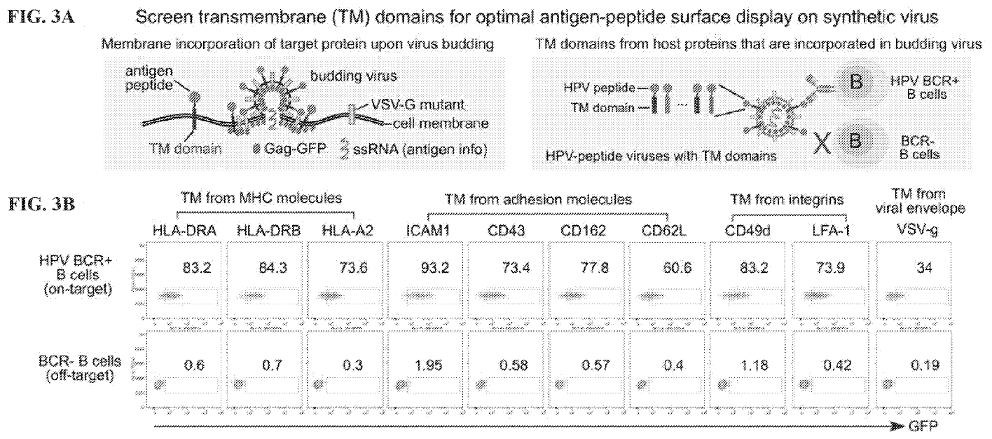

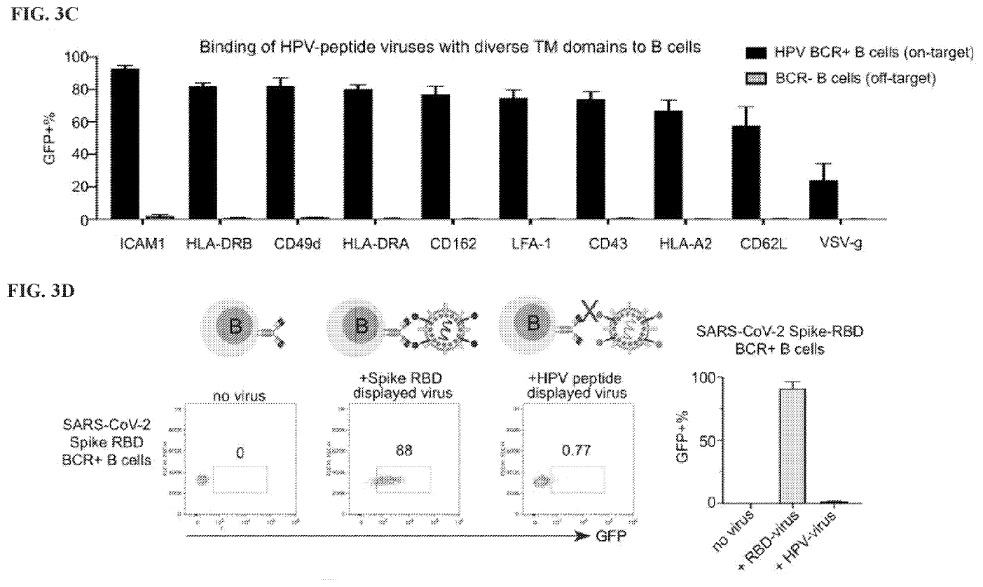

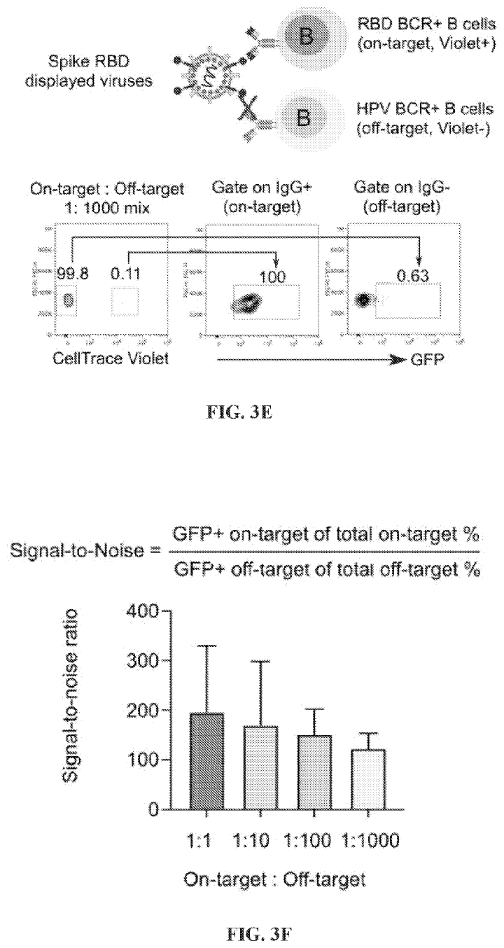

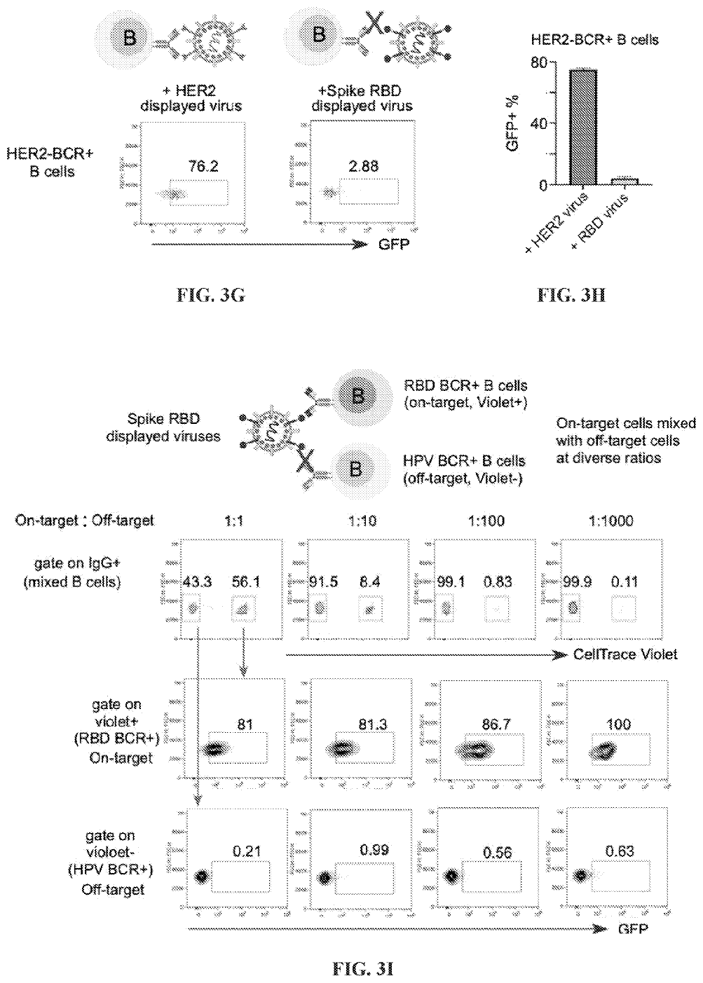

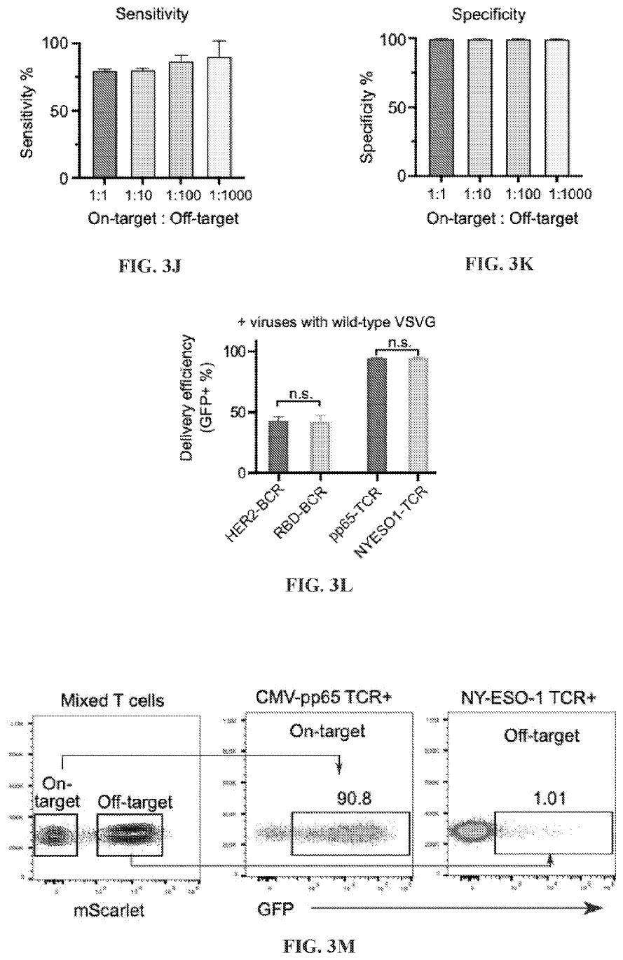

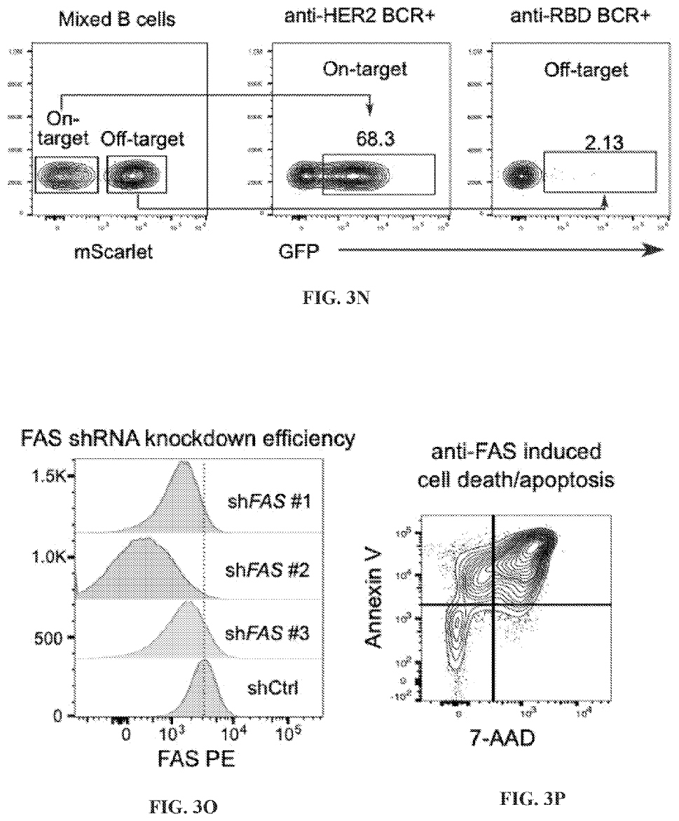

Unlike TCR recognition of antigen peptides that are presented by MHC on cell surface, BCR can recognize antigen epitopes that are derived from not only cell surface proteins but also intracellular proteins, extracellular, and secreted proteins. The main challenge of ENTER to decode B cell specificity is to display B cell antigens which do not contain their native transmembrane (TM) domains on the viral surface. To display antigen epitopes from intracellular proteins on viral surface, Applicants sought to engineer a TM domain for optimal surface display of B cell antigens. To select candidates of the TM domain for viral surface display, Applicants took advantage of the unique ability of HIV-1 viruses to incorporate host proteins on viral surface during virus budding. Nascent HIV-1 viruses can selectively incorporate certain host TM proteins while excluding other abundant host surface proteins during viral assembly and budding process (Burnie and Guzzo, 2019). Applicants prioritized a list of highly abundant host TM proteins that are incorporated into viral surface from previous literature using mass spectrometry of viruses, immunocapture assay, and flow virometry method (Burnie et al., 2020; Cantin et al., 1996; Chertova et al., 2006; Grover et al., 2015; Jalaguier et al., 2015). This list of host TM proteins included MHC class I and II molecules (HLA-DRA, HLA-DRB, HLA-A2), adhesion molecules (ICAM1, CD43, CD162, CD62L), and integrin family members (CD49d, LFA-1) (see, e.g., FIG. 3A).

To determine the specificity and efficiency of viral display of B cell epitopes with these diverse TM domains, viruses were engineered to express a B cell antigen epitope derived from human papillomavirus (HPV) minor capsid antigen L2 (HPV16 L2 residue₁₇₋₃₆) and fused with TM domains from the prioritized list. Next, a BCR expressing B cell line was generated that specifically targets the HPV16 L2 B cell epitope (Wang et al., 2015). After incubating the TM domain fused and B cell epitope displayed viruses with B cells either expressing HPV-BCR (on-target) or without any BCR (off-target), the percentage of GFP+B cells was quantified to measure the efficiency and specificity (see, e.g., FIG. 3B). In addition to TM domains from host proteins, the viruses were further engineered to fuse the B cell epitope with the TM domain from the fusogen VSV-G, a viral envelope protein that can be assembled in budding viruses. The results revealed ICAM1 TM domain as the top candidate since over 90% of HPV antigen-specific BCR+ B cells were GFP+(see, e.g., FIGS. 3B-3C). This is consistent with previous reports showing ICAM1 is selectively acquired in budding viruses through interaction with viral matrix protein (Jalaguier et al., 2015).

To test if the ICAM1 TM domain can be applied to present other B cell antigens in addition to the linear epitopes from HPV16, the viruses were engineered to display Receptor Binding Domain (RBD) from SARS-CoV-2 spike protein (see, e.g., FIG. 3D). The result showed that 88% of spike-RBD BCR+ B cells were labeled by RBD displayed viruses, indicating that ENTER with optimized TM domain can be applied to decode B cell specificity for both linear epitopes (HPV16 L2₁₇₋₃₆ antigens) and full antigen domains (SARS-CoV-2 spike RBD).

Beyond the capability of ENTER to display intracellular and extracellular B cell antigens, additional experiments were performed to determine if ENTER can decode B cell specificity towards cell surface B cell antigens. ENTER was engineered to display HER2 using its native TM domain. HER2 is an epidermal growth factor receptor, which is overexpressed in breast cancer cells (Gutierrez and Schiff, 2011). The experimental data showed that 76% of anti-HER2 BCR B cells were detected by the HER2 displayed viruses (see, e.g., FIGS. 3G-3H), highlighting the generalization of ENTER to display any B cell antigens from intracellular (HPV L2), extracellular (Spike RBD), and cell surface proteins (HER2).

In addition, to further examine the specificity and sensitivity of ENTER to decipher interactions between BCR and B cell antigens, on-target B cells (with BCR recognizing SARS-CoV-2 Spike RBD antigen) and off-target B cells (with BCR recognizing HPV L2 antigen) were mixed at different ratios and then incubated with the Spike RBD antigen presented viruses (see, e.g., FIGS. 3E and 3I). To distinguish these on-target and off-target B cells, the on-target B cells with cell trace violet dye. The signal-to-noise ratio was calculated based on the frequency of on-target GFP+ cells versus that of off-target GFP+ cells. The signal-to-noise ratio was around 100- to 200-fold (see, e.g., FIG. 3F), indicating a profound specificity and sensitivity of the TM domain optimized viruses to display B cell antigen epitopes (see, e.g., FIGS. 3J-3K).

Taken together, the experimental data described above demonstrates that ENTER is a platform that can successfully capture the interaction of BCR and antigen in a highly specific and sensitive manner.

### Example 4

This Example describes experiments performed to investigate if ENTER is capable to delete or expand antigen-specific T or antigen-specific B cells by targeted cargo delivery.

First, to test the antigen specificity of cargo delivery, GFP transgene was used as a cargo to measure the delivery efficiency and specificity. Upon infection of lentiviruses pseudotyped with wild-type VSV-G, comparable transduction efficiency irrespective of TCR or BCR specificity was observed (see, e.g., FIG. 3L). Additional experiments were then carried out to engineer viruses displaying pp65₄₉₅₋₅₀₃ pMHC ligand and VSV-G-mutant fusogen, carrying GFP transgene on the viral RNA, and other viral components including wild-type integrase (see, e.g., General Methods in Example 11). After infection of pp65₄₉₅₋₅₀₃ pMHC viruses with CMV-pp65 TCR+ T cells targeting pp65₄₉₅₋₅₀₃ and NY-ESO-1 TCR+ T cells targeting irrelevant antigen, it was observed that 82% of on-target TCR-T cells expressed GFP whereas only 0.22% of off-target TCR-T cells were GFP+(see, e.g., FIGS. 4A-4B). Similarly, additional experiments were carried out to engineer viruses displaying B cell antigen HER2 and carrying GFP transgene as a cargo (see, e.g., FIG. 4C). The experimental data showed specific delivery of GFP transgene in HER2 BCR+ B cells but not RBD BCR+ B cells (see, e.g., FIG. 4D).

Next, to examine if ENTER-mediated targeted gene delivery can exert proper function in antigen-specific T or B cells, additional experiments were performed to engineer pMHC displayed viruses to carry herpes simplex virus thymidine kinase (HSV-TK) gene, a well-established suicide gene in response to drug ganciclovir (GCV) (Beltinger et al., 1999). CMV-pp65 TCR+ T cells were mixed with NY-ESO-1 TCR+ T cells which express mScarlet in a 1:1 ratio, and then pp65₄₉₅₋₅₀₃ displayed viruses carrying suicide gene were added. Three days post infection, GCV drug was added to kill cells expressing HSV-TK and the cell survival was monitored for 4 days (see, e.g., FIG. 4E). A specific cargo delivery in on-target T cells among a pool of mixed T cells post viral infection was observed (see, e.g., FIG. 3M). After 4 days of GCV treatment, a specific depletion of on-target T cells (CMV-pp65 TCR+) without impacting the off-target T cells was observed see, e.g., (see, e.g., FIG. 4F). To determine if the targeted killing is not induced by viral infection, viruses with GFP gene as a negative control were generated. It was found that HSV-TK gene delivery resulted in a significant ˜8-fold depletion of on-target T cells compared to GFP gene delivery (see, e.g., FIG. 4G), further validating that selective depletion of one T cell clone is achieved by antigen-specific suicide gene delivery. To test if antigen-specific gene delivery can be applied to B cells, HER2 BCR+ B cells were mixed with RBD BCR+ B cells expressing mScarlet, followed by addition of HER2 displayed viruses carrying suicide gene (see, e.g., FIGS. 4H and 3N). Similarly, after GCV drug treatment, the result showed a significant reduction of on-target B cells with suicide gene delivery compared to control GFP gene (see, e.g., FIGS. 4I-4J), suggesting that ENTER enables selective depletion of one B cell clone among a pool of B cells by antigen-specific suicide gene delivery.

Conversely, additional experiments were also performed to examine if ENTER enables selective survival and retention of antigen-specific T cells. An aim of these experiments was to deliver short hairpin RNA (shRNA) against the cell death receptor FAS in antigen-specific T cells to prevent the FAS-induced programmed cell death (Yonehara et al., 1989). After screening multiple shRNAs targeting FAS, shFAS #2 was selected with the best knockdown efficiency reflected by a dramatic reduction of surface expression of FAS compared to control shRNA (shCtrl) group (see, e.g., FIG. 3O). Further generated were pp65₄₉₅₋₅₀₃ displayed carrying shFAS #2 or shCtrl and infected a mixed pool of on-target (CMV-pp65 TCR+) and off-target (NY-ESO-1 TCR+) T cells with these viruses (see, e.g., FIG. 4K). The result showed a significant decrease of FAS protein surface expression in on-target T cells infected with shFAS #2 compared to shCtrl group or off-target uninfected T cells, indicating a targeted shRNA delivery (see, e.g., FIG. 4L). Next, these cells were treated with anti-FAS antibody to trigger FAS-mediated cell death revealed by Annexin V and 7-AAD staining (see, e.g., FIGS. 4K and 3P). After normalizing on-target versus off-target population, a significant increase was observed on on-target T cells among live cells after FAS knockdown compared to shCtrl group (see, e.g., FIG. 4M). Together, the data described herein showed that ENTER permits targeted cargo delivery to manipulate complex cellular populations with ligand-receptor specificity.

### Example 5

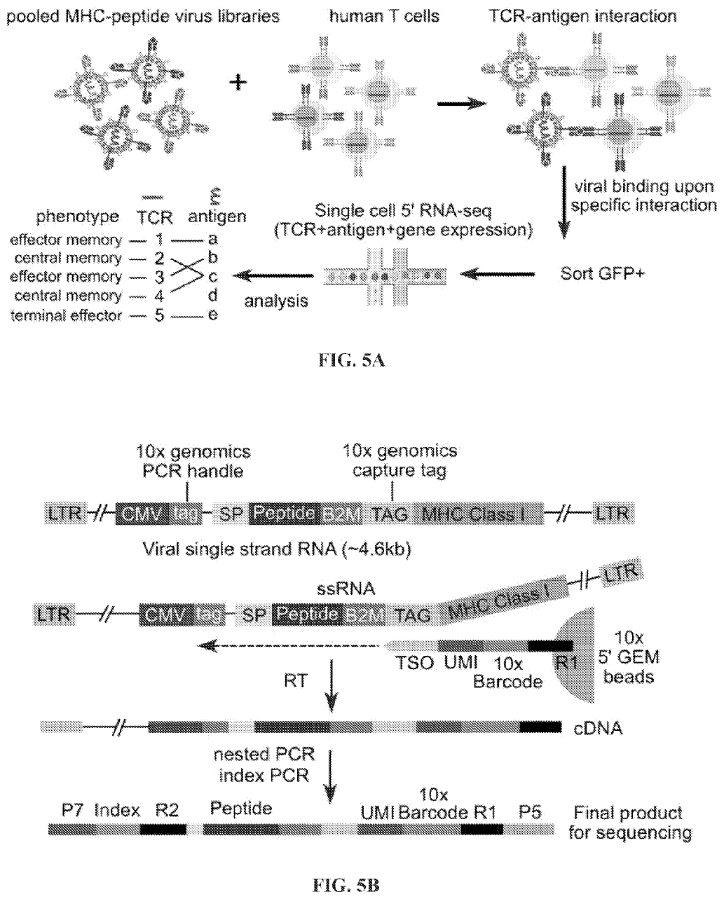

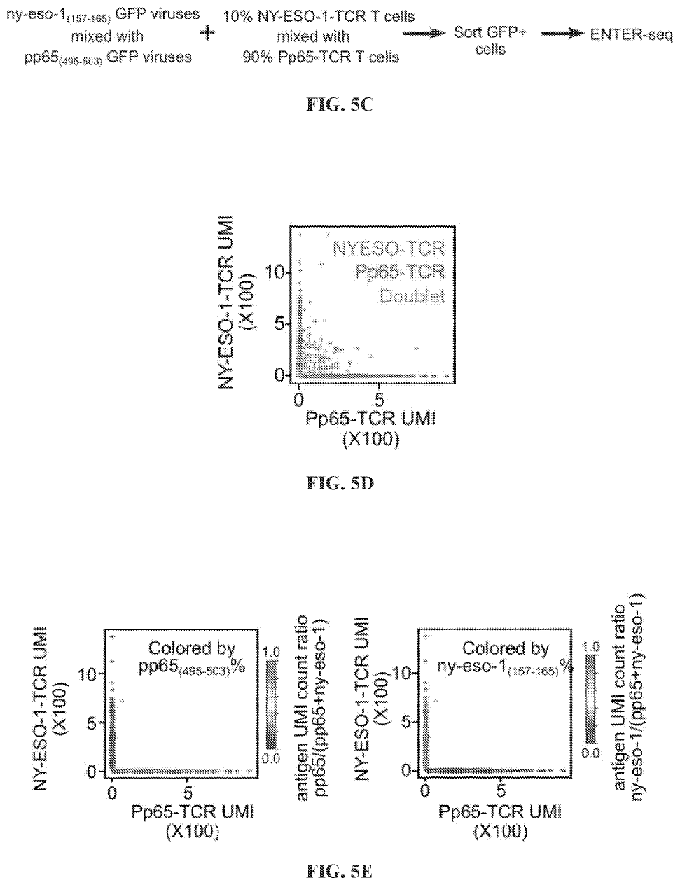

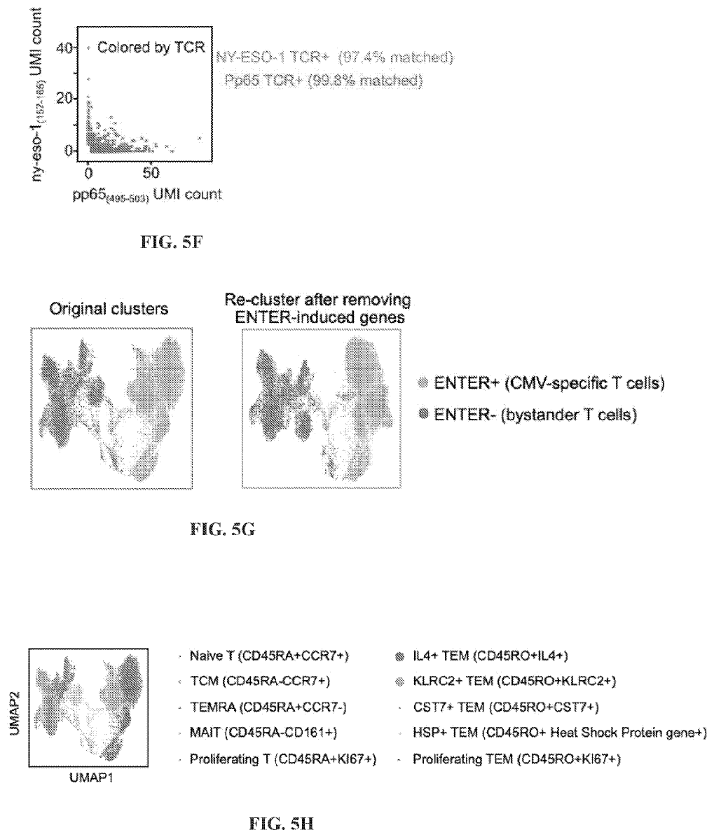

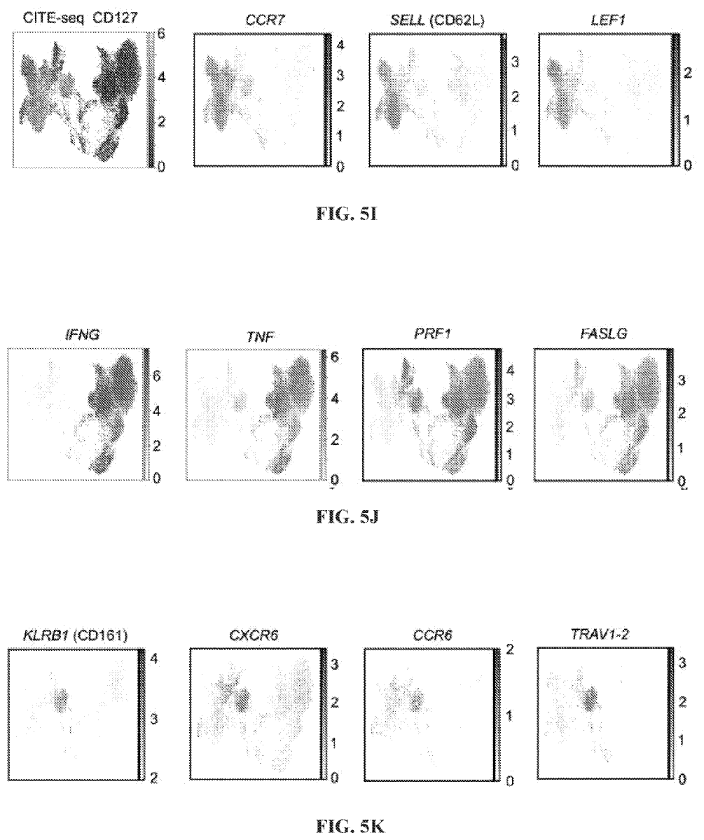

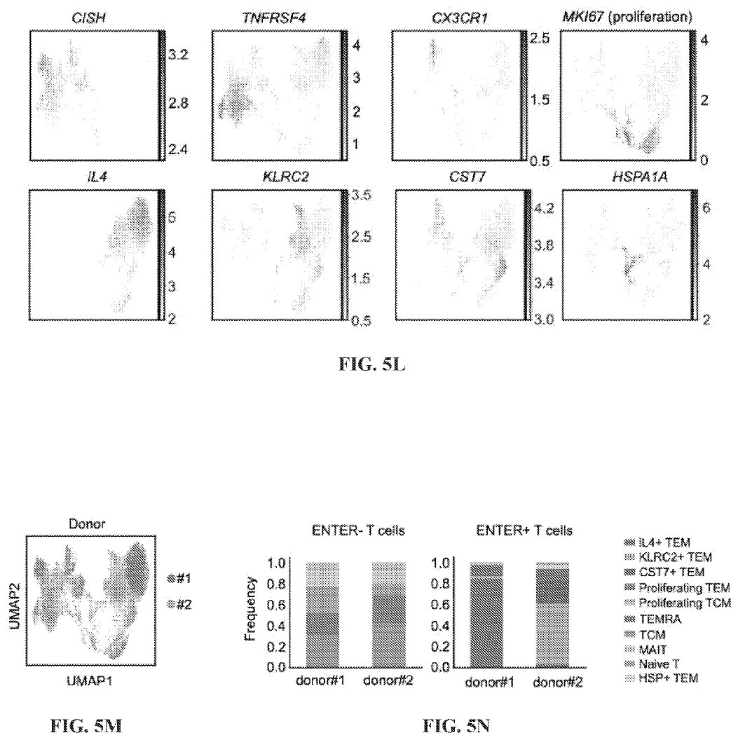

This Example described the results of experiments in which the viral display platform ENTER was combined with droplet-based single-cell RNA-seq to develop ENTER-seq, a technology for capturing the ligand-receptor interaction and molecular blueprints at the single-cell resolution. Thus ENTER-seq captures MHC-peptide antigen specificity, TCR repertoire and gene expression profile at single-cell resolution. In some embodiments, the workflow of ENTER-seq comprises (1) generation of a pooled pMHC displayed GFP viruses, (2) incubation of these viruses with human T cells, (3) sorting virus-labeled GFP+ cells for droplet-based single cell profiling (e.g., 5 prime single-cell RNA-seq/V(D)J-seq by 10× Genomics), (4) generation and sequencing of three single-cell libraries including gene expression, V(D)J TCR repertoire, and antigen-peptide sequence (see, e.g., FIG. 5A).

The single-chain PMHC information is stored in viral single-strand RNAs (ssRNAs) that are packaged into lentiviral particles. The viral ssRNA is approximately 4.6 kb, making it difficult to reverse transcribe (RT) into full-length cDNA in droplets. To efficiently capture the pMHC information on viral RNA during the RT step in each droplet, a capture tag was inserted in the linker region between B2M and MHC, and another PCR handle next to CMV promoter (see, e.g., FIG. 5B). This capture tag allows capture by commercially available 5′ GEM beads through hybridizing with the Template Switch Oligo (TSO) sequence conjugated on the beads. The PCR handle permits convenient amplification of targeted peptide sequence without spiking in additional primers during the cDNA amplification step (see, e.g., FIG. 5B). Further nested PCR and index PCR allow targeted enrichment of the antigen peptide sequence to generate the final antigen library for deep sequencing. The insertion of the capture tag and PCR handle does not affect the display of pMHC on viruses and specific interaction with TCR-expressing T cells (see, e.g., FIGS. 4N-4O).

To benchmark ENTER-seq for single-cell profiling of antigen specificity and TCR repertoire, ENTER-seq was performed on a mixed TCR-expressing T cells with a pooled pMHC displaying viruses. To mimic a real-life T cell population, 10% of T cells was mixed with TCR recognizing ny-eso-1₁₅₇₋₁₆₅ antigen and 90% of T cells with TCR recognizing CMV pp65₄₉₅₋₅₀₃ antigen, and then incubated with pooled viruses displaying ny-eso-1₁₅₇₋₁₆₅ antigen or pp65₄₉₅₋₅₀₃ antigen (see, e.g., FIG. 5C). Analysis of the unique TCR sequence after filtering out doublets confirmed the mixing ratio of T cells at 9.4% (ny-eso-1₁₅₇₋₁₆₅-TCR+) vs 90.6% (CMV pp65₄₉₅₋₅₀₃-TCR+), which is similar to the input mixing ratio (10% vs 90%) (see, e.g., FIG. 5D). A total of 4198 T cells were further recovered with reliable antigen peptide information and TCR sequence after filtering the unique molecular identifier (UMI) count of TCRs and antigen peptides (Method). The ratio of the UMI from the dominant antigen peptide among total peptides was calculated. A high concordance of antigen peptides to their paired TCR was observed (see, e.g., FIG. 5E). After matching TCR sequences to the antigen peptides at the single-cell level, the result showed that 99.8% of pp65₄₉₅₋₅₀₃+ cells and 97.4% of ny-eso-1₁₅₇₋₁₆₅+ cells were matched with their corresponding TCR sequences respectively (see, e.g., FIG. 5F).

Thus, the experimental data described above illustrates that ENTER-seq can sensitively and robustly capture the interaction of TCR repertoire and cognate HLA antigen peptide at the single-cell resolution.

### Example 6

This Example describes the results of experiments performed to illustrate that optimized ENTER-seq detects rare antigen-specific primary human T cells. Particularly, ENTER-seq can be applied to the rare antigen-specific primary T cells directly isolated from human blood.

The sensitivity of the ENTER-seq system was first validated using GFP viruses displaying CMV-pp65 antigen epitope presented on HLA-A2 allele, and primary T cells from HLA-A2+ patients with a history of CMV infection. Primary T cells were incubated with pp65₄₉₅₋₅₀₃ antigen displayed viruses and then stained with a widely used CMV pp65₄₉₅₋₅₀₃ tetramer which serves as a positive control. The tetramer staining analysis showed that 1% of the T cells were pp65₄₉₅₋₅₀₃ antigen-specific (see, e.g., FIG. 4Q). 83% of the pp65₄₉₅₋₅₀₃ tetramer-positive T cells were labeled by GFP viruses. To further increase the sensitivity of detection by flow cytometry, the GFP was replaced with mNeon, a monomeric green fluorescence protein that is substantially brighter than GFP (see, e.g., FIGS. 4P-4Q). Indeed, 98% of pp65₄₉₅₋₅₀₃ tetramer-positive T cells were recovered by mNeon viruses displaying pp65₄₉₅₋₅₀₃ epitope, but not negative control viruses, indicating a significantly higher efficiency than GFP viruses (see, e.g., FIGS. 4Q-4S).

### Example 7

This Example describes the results of experiments performed to illustrate that ENTER-seq of peptide-enriched CMV-specific T cells can uncover donor-specific immunogenic CMV epitopes and antigen-specific molecular phenotype.

Anti-viral T cells are essential to control viral replication and dissemination. Adoptive transfer of in-vitro expanded CMV-specific T cells has shown great efficacy to control CMV infection in patients receiving transplantation. However, it is largely unexplored how CMV peptide-induced antigen-specific expansion in vitro impacts the molecular phenotype, clonal expansion and potential function of CMV-specific T cells.

In these experiments, ENTER-seq was used to characterize the transcriptional program, antigen specificity and TCR clonality of CMV-specific T cells expanded via CMV antigen peptide stimulation. To enrich and expand CMV-specific T cells, human peripheral mononuclear cells (PBMCs) from CMV seropositive donors were first cultured with a pool 12 CMV antigen peptides for 10 days (Lehmann et al., 2020; Lübke et al., 2020; Solache et al., 1999) (see, e.g., FIG. 4T). The peptides were processed and presented by autologous antigen-presenting cells which then stimulated CMV antigen-specific T cells for later expansion. To test the specificity of the ENTER viruses of the present disclosure on peptide-enriched T cells, T cells were expanded using peptide pp65₄₉₅₋₅₀₃ and then incubated with pp65₄₉₅₋₅₀₃ antigen presented mNeon viruses, followed by staining with pp65₄₉₅₋₅₀₃ tetramer. Flow cytometry analysis showed that ˜99% of tetramer+ T cells were labeled by viruses (see, e.g., FIG. 4U), demonstrating a high specificity and sensitivity of ENTER to detect peptide enriched antigen-specific T cells.

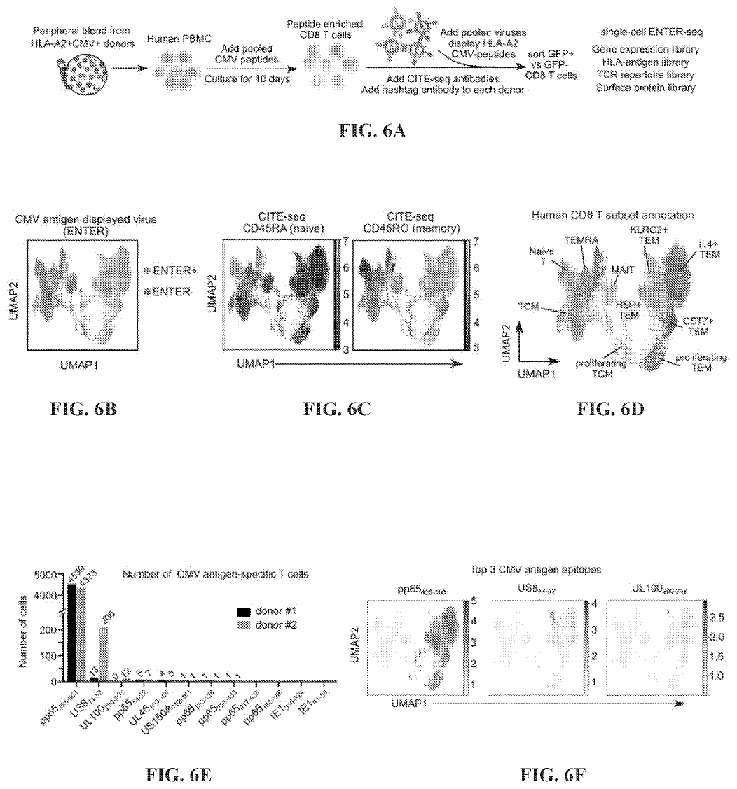

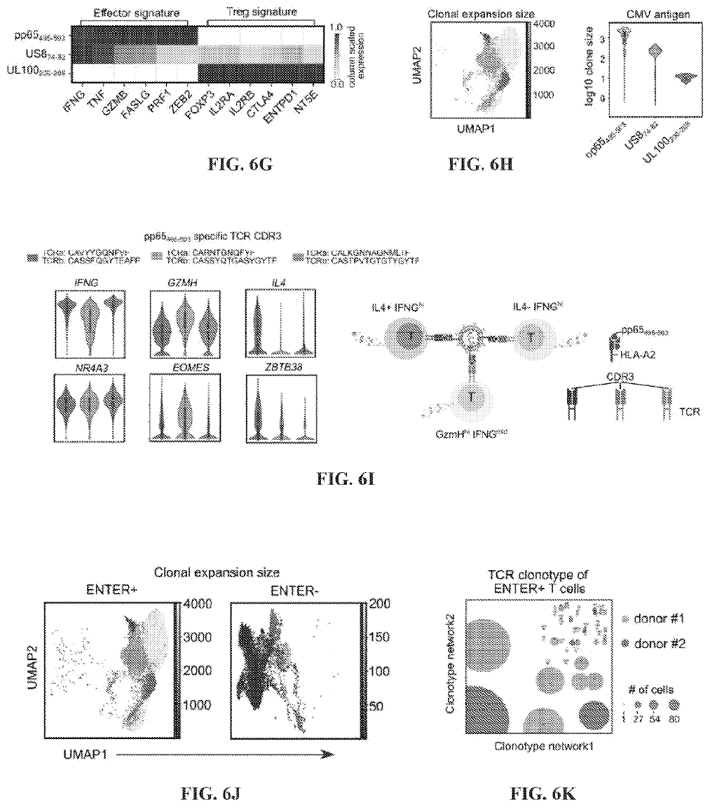

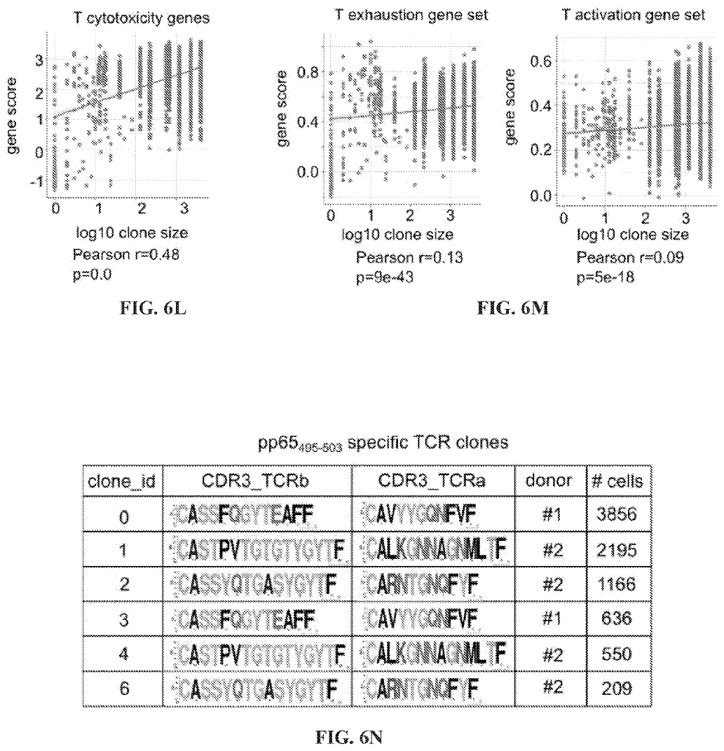

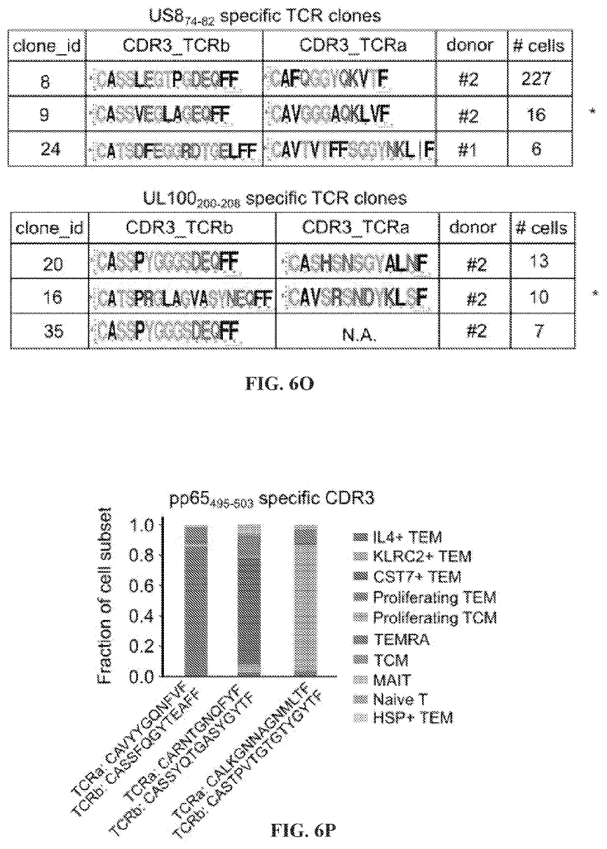

A pool of ENTER viruses displaying these 12 CMV antigen epitopes were prepared and incubated with expanded T cells from 4 different CMV seropositive HLA-A2 positive donors (see, e.g., Figure S4G). Dramatic expansion of CMV antigens-specific T cells was observed in 2 out of 4 donors (19.4% in donor #1 and 8.78% in donor #2) (see, e.g., FIG. 4V). Next, ENTER-seq was performed on expanded T cells from these two donors, and labeled each donor sample with a unique hashtag antibody. To further interrogate the phenotype of antigen-specific T cells, ENTER-seq was combined with CITE-seq through staining cells with DNA barcoded antibodies targeting cell surface proteins CD45RA, CD45RO, and IL7R (see, e.g., FIG. 6A).

After sorting GFP+(CMV antigen-specific T cells) and GFP− (bystander T cells) CD8+ T cells followed by droplet-based single cell capture, libraries were generated to profile gene expression programs, CMV antigen peptides, TCR repertoires, and surface proteins (including CITE-seq proteins and hashtag proteins) in individual cells. The result showed that cells that bound with CMV antigen displayed viruses (ENTER+) were phenotypically different from cells without virus binding (ENTER−) (see, e.g., FIG. 6B). To test if such phenotypic difference is induced by the binding of ENTER viruses, RNA-seq data of CMV pp65 TCR-T cells was compared with incubation of pp65₄₉₅₋₅₀₃ displayed ENTER viruses or pp65₄₉₅₋₅₀₃ tetramers. In summary, 28 genes were identified as being differentially expressed between ENTER group and tetramer group (2-fold change and adjusted P value<0.01) (see, e.g., FIG. 4W). Notably, all 28 genes were significantly upregulated in ENTER virus-treated cells and were mainly associated with TCR activation (CD69) and transcription factors induced by TCR signaling (FOS, NR4A1, NR4A3, EGR1, etc.) (see, e.g., FIG. 4W). This result suggested that the binding of pMHC displayed viruses may weakly induce TCR activation of resting/naïve CMV pp65 TCR-T cells compared to tetramer binding. To further determine if this ENTER-induced gene upregulation leads to the phenotypic difference between CMV-specific T cells and bystander T cells, Leiden clustering was performed with or without ENTER-induced gene signature (see, e.g., General Methods in Example 11). A clear separation was found between the CMV-specific T cells and bystander T cells after removal of those 28 genes, suggesting that the phenotypic difference is not caused by the binding of ENTER viruses (see, e.g., FIG. 5G).

After integrating surface protein landscape and gene expression, it was observed that peptide-enriched CMV-specific T cells (ENTER+) were mainly effector memory T (TEM) cells (CD45RO+CD45RA−) while ENTER− cells were a mixture of naïve and central memory T (TCM) cells (see, e.g., FIGS. 6C and 511-5I). Compared to ENTER− cells, ENTER+ cells were potentially protective T cells based on high expression of effector molecules such as IFNG, TNF, and cytotoxic molecules including granzymes and perforin (see, e.g., FIG. 5J). The single cell RNA-seq data clustered all T cells into 10 clusters including: (1) naïve T cells: CD45RA+CCR7+; (2) TCM: CD45RA-CCR7+; (3): terminally differentiated effector T cells (TEMRA): CD45RA+CCR7−; (4) mucosal-associated invariant T cells (MAIT): CD45RA-CD161+CXCR6+; (5) proliferating T cells: CD45RA+KI67+; (6) IL4+ TEM: CD45RO+IL4+; (7) KLRC2+ TEM; CD45RO+KLRC2+; (8) CST7+ TEM (CD45RO+CST7+; (9): HSP+TEM: CD45RO+ heat shock protein (e.g. HSPA1A)+; (10): proliferating TEM: CD45RO+KI67+(see, e.g., FIGS. 6D and 5H-5L). Comparison of subset frequency between donors showed that ENTER− bystander T cells were relatively similar between two donors while ENTER+ CMV antigen-specific T cells were phenotypically different between two donors, suggesting that two donors may have different immune responses to CMV antigens (see, e.g., FIGS. 5M-5N).

To determine if there is a donor-specific immune response to CMV antigens, the number of T cells recognizing specific CMV antigen epitopes in each donor was measured. Pp65₄₉₅₋₅₀₃-specific T cells were the most dominant antigen-specific T cells in both donors, suggesting pp65₄₉₅₋₅₀₃ is the most common and immunogenic CMV antigen (see, e.g., FIG. 6E). This is consistent with previous reports showing a high frequency of CMV pp65₄₉₅₋₅₀₃ specific T cells across many donors (Elkington et al., 2003; Gillespie et al., 2000; Wills et al., 1996). Also observed was a higher frequency of US8₇₄₋₈₂- and UL100₂₀₀₋₂₀₈-specific T cells in donor #2 compared to donor #1, indicative of donor-specific viral epitope immunogenicity. Interestingly, when projecting the top 3 antigen epitopes to gene expression UMAP plots, 3 distinct clusters were observed, suggesting different epitopes may drive unique gene CD8+ T cell fates and expression programs (see, e.g., FIG. 6F). Indeed, pp65₄₉₅₋₅₀₃-specific T cells have high expression of effector cytokines (e.g. IFNG, FASLG, PRF1, etc.) and transcription factors essential for effector T cells (e.g. ZEB2) (see, e.g., FIG. 6G). Surprisingly, UL100₂₀₀₋₂₀₈-specific T cells have high expression of FOXP3, IL2RA(CD25), and CTLA4, which are characteristics of regulatory T (Treg) cells (Billerbeck et al., 2007; Churlaud et al., 2015; Fontenot et al., 2003; Wing et al., 2008), indicating that UL100₂₀₀₋₂₀₈-specific T cells resemble CD8+ Treg cells (see, e.g., FIG. 6G) (Vieyra-Lobato et al., 2018). Thus, ENTER-seq not only uncovers donor-specific viral epitopes but also reveals distinct molecular blueprints of antigen-specific T cells upon recognizing different antigen epitopes from the same virus.

### Example 8

### Inter-Clonal Phenotypic Diversity Underlying the Same Antigen Specificity

This Example describes the results of experiments performed to illustrate inter-clonal phenotypic diversity underlying the same antigen specificity. To investigate the clonal expansion of CMV antigen-specific T cells, an integrative analysis of TCR repertoire, antigen specificity and gene expression at the single cell level was performed.

In these experiments, TCR clonotypes were defined by the identity of CDR3 nucleotide sequences (Yassai et al., 2009). Peptide enriched CMV-specific T cells (ENTER+) exhibited high clonal expansion (maximum 3856 cells per TCR clone) compared to bystander T cells (ENTER−, maximum 174 cells per TCR clone) (see, e.g., FIG. 6J). Using dominant TCR clonotype as references, low false-negative rate (FNR<3%) and false-positive rate (FPR<1%) was calculated and observed, which highlights the high sensitivity and specificity of ENTER-seq on primary T cells (see, e.g., General Methods in Example 11). Next, additional experiments were performed to examine if there is any overlap of CMV antigen-specific TCR clones between two donors. The result of these experiments showed that the two donors have unique expanded TCR clones without any overlap (see, e.g., FIG. 6K), consistent with prior studies showing that antigen-specific TCR clonotypes are usually private to each individual due to the high diversity of TCR repertoire (Dupic et al., 2021; Robins et al., 2009). Thus, the shared TCR specificity could not have been predicted from TCR sequences alone but required pMHC binding data. Further integrative analysis of antigen specificity and TCR clonal expansion showed that different CMV antigen epitope-specific T cells exhibit distinct behavior of TCR clonal expansion (see, e.g., FIG. 6H). The clonal expansion size of pp65₄₉₅₋₅₀₃-specific T cells was significantly larger than that of UL100₂₀₀₋₂₀₈-specific T cells (see, e.g., FIG. 6H). Highly expanded TCR clones were associated with high expression of cytotoxicity genes, e.g. comparing pp65₄₉₅₋₅₀₃-specific T cells to UL100₂₀₀₋₂₀₈-specific T cells. Indeed, across all clonotypes, the expression of genes associated with cytotoxicity was significantly correlated with TCR clonal expansion (Pearson correlation r=0.48, p=0.0, FIG. 6L). In contrast, the correlation between clonal expansion with other gene signatures was relatively weak (r=0.13 for T cell exhaustion genes, r=0.09 for T cell activation genes (see, e.g., FIG. 6M).

Because multiple TCR nucleotide sequences can encode the same CDR3 amino acid sequences which target the same antigen epitope, the clonotypes were then merged based on identical CDR3 amino acid sequences for each CMV antigen epitope (see, e.g., FIG. 6N-6O). For the most immunogenic CMV epitope pp65₄₉₅₋₅₀₃, three dominant CDR3 clonotypes were identified. Among them, two TCR beta chain sequences (CASSFQGYTEAFF; SEQ ID NO: 54 and CASSYQTGASYGYTF; SEQ ID NO: 55) are identical with published pp65₄₉₅₋₅₀₃-specific TCRs in the IEDB database, further validating the specificity of the ENTER platform disclosed herein (see, e.g., FIG. 6O). When combining CDR3 clonotypes with gene expression profiles in pp65₄₉₅₋₅₀₃ specific T cells, it was discovered that different clonotypes exhibited distinct gene expression phenotype including distinct cytokine profiles, cytolytic enzyme, and transcription factor expression, which indicates an inter-clonal phenotypic diversity that targets the same antigen epitope (see, e.g., FIGS. 6I and 6P). Thus, ENTER-seq can functionally characterize both the TCR binding specificity and the TCR-associated cell states.

### Example 9

This Example describes the results of experiments demonstrating that ENTER-seq of primary CMV-specific T cells from patients reveals intra-clonal diversity in genes associated with cytotoxicity and type-I IFN response.

To decode the anti-viral T cell memory in CMV seropositive patients, ENTER viruses were engineered for displaying top 3 CMV antigen epitopes identified previously and performed ENTER-seq on primary T cells isolated directly from patient blood without in vitro expansion (see, e.g., FIG. 7A). Integrative analysis of CITE-seq and gene expression profiles showed that CMV-specific T cells (ENTER+) in patients were mainly terminally differentiated effector memory T cells (TEMRA, CD45RO-CD45RA+CCR7−) (see, e.g., FIGS. 7B-7C). This observation is consistent with previous studies showing the accumulation of TEMRA CMV-specific T cells in CMV seropositive patients (Appay et al., 2002; Derhovanessian et al., 2011).

After subset clustering, heterogeneity of TEMRA populations was observed in CMV-specific T cells with diverse patterns of gene expression associated with cytotoxic function, chemokines, costimulatory/coinhibitory molecules, and type-I IFN response (see, e.g., FIGS. 7D and 7L). For example, in CMV-specific T cells, TEMRA #1 cluster contains high expression of cytotoxic genes like IFNG, TNF, and PRF1 but not GZMK whereas TEMRA #4 cluster is IFNG-TNF-PRF1+GZMK+ (see, e.g., FIGS. 7C and 7L). Notably, TEMRA #2 cluster in CMV-specific T cells contains low expression of all cytotoxic genes but high expression of type-I IFN stimulated genes (ISG) such as ISG15, ISG20, IFIT1, and OASL etc. (see, e.g., FIGS. 7C and 7L). Such upregulation of ISG genes reflected a specific induction of type-I IFN response in a small subset of CMV-specific T cells, which might be stimulated by local production of type-I IFN responding to CMV viruses or bystander production of type-I IFN from other pathogens in patients. To test if the ENTER virus binding influences the T cell state (e.g., type-I IFN response), Leiden clusterings with or without ENTER-induced genes were compared, and highly concordant clusters were observed. This result suggested that the binding of ENTER viruses have minimal impact on the T cell state of primary T cells isolated from patient blood (see, e.g., FIG. 7M).

Subsequently, antigen specificity, TCR repertoire, and gene expression of CMV-specific T cells in patients were integrated. As expected, CMV-specific T cells were found to predominantly pp65₄₉₅₋₅₀₃-specific T cells associated with a wide range of clonal expansion, confirming that pp65₄₉₅₋₅₀₃ is a highly immunogenic CMV epitope (see, e.g., FIGS. 7E-7F). Further observed was a rare population of T cells (43 cells) that were labeled by US8₇₄₋₈₂ pMHC ENTER viruses (see, e.g., FIG. 7N). TCR sequencing revealed that up to 60% of the US8₇₄₋₈₂-specific cells at rest share TCRs with peptide-expanded US8₇₄₋₈₂-specific T cells, confirming their clonal identity (see, e.g., FIG. 7O). Interestingly, by tracking dominant TCR clones in each donor along the expansion, it was found that donor #2 contain the most expanded TCR clone, whereas the TCR clone in donor #1 rarely expand (see, e.g., FIGS. 7P-7Q). These experimental data demonstrate the ability of ENTER-seq to detect multiple antigen specificities in the presence of highly dominant epitope from fresh PBMCs without expansion.

The number of pMHC bound per cell was also quantified to measure the binding strength of pMHC displayed ENTER viruses. By integrating the pMHC binding strength and TCR clonal expansion of CMV pp65-specific T cells in patients, the result showed significantly higher binding of pMHC in highly expanded T cell clones (clone size >50) than lowly expanded T cells (see, e.g., FIG. 7G). Because ENTER pMHC binding positively correlated with TCR affinity (see, e.g., FIG. 2F), the experimental data described herein suggested that high TCR affinity is associated with and likely drives greater T cell clonal expansion.

Among the pp65₄₉₅₋₅₀₃-specific T cells, 3 dominant TCR clones were found with same TCR sequence as peptide-enriched pp65-specific T cells. Similar to in vitro peptide-expanded T cells, these pp65-specific TCR clones exhibit phenotypic difference although they target the same antigen epitope (see, e.g., FIG. 7R). Strikingly, a phenotypic heterogeneity in the same TCR clones was observed (see, e.g., FIGS. 7R-7S). For example, TCR clone #2 is composed of type-I IFN ISG+ TEMRA, stressed IFNG^{hi}FASLG^{lo} TEMRA, and IFNG^{lo}FASLG^{hi} TEMRA (see, e.g., FIGS. 7R-7S). This data revealed an intra-clonal phenotypic diversity underlying the same TCR clones, suggesting that the T cell state is impacted by both TCR binding specificity and local microenvironment.

### Example 10

This Example describes the results of experiments performed to demonstrate phenotypic transition and clonal divergence of CMV-specific T cells upon ex vivo antigen peptide-induced expansion.

To understand how antigen-specific expansion impacts the molecular phenotypes of anti-viral T cells, a comparison of ENTER-seq of CMV-specific T cells prior and post peptide-induced expansion was carried out. By combining antigen specificity, CITE-seq, and transcriptional programs, it was observed that a phenotypic transition of pp65-specific T cells occurred upon antigen-specific expansion (see, e.g., FIG. 7H). pp65-specific T cells isolated directly from patient blood were mostly TEMRA T cells, a terminally differentiated effector memory subset. After antigen-induced expansion, these T cells lost CD45RA expression and gained CD45RO expression, suggesting that these TEMRA T cells can further differentiate into effector memory T cells (TEM, CD45RA−CD45RO+) (see, e.g., FIG. 7H). This observation was validated in both donors by flow cytometry (see, e.g., FIG. 7I).

Additional experiments were performed to examine if the phenotypic change upon expansion is driven by the cell state transition of the entire antiviral repertoire or biased by selective expansion of specific T cell clones. In these experiments, TCR sequence was utilized as a “natural” barcode to track the cell state of each dominant T cell clone prior and post expansion. The IFN-I ISG gene score and cytotoxicity gene score were calculated to reflect cell state in type-I IFN stimulation and cytotoxic function of individual T cell (see, e.g., General Methods in Example 11). It was found that the IFN-I ISG and cytotoxicity scores were highly heterogenous at rest in each T cell clone in patients (see, e.g., FIG. 7J). After ex vivo expansion, the IFN-I ISG and cytotoxicity scores became remarkably focused across T cell clones: a loss of type-I IFN ISG gene expression and upregulation of cytotoxic genes were found in all three clones (see, e.g., FIG. 7J). This result suggested that peptide induced expansion may further boost the effector function of T cells whereas the type-I IFN response observed in patients cannot be maintained upon ex vivo expansion.

Conversely, antigenic activation can also evoke inter-clonal phenotypic diversity. Among three pp65₄₉₅₋₅₀₃-specific T cell clones, all three had low expression of T helper 2 (Th2) cytokine gene IL13 but a spectrum of expression of the memory T cell transcription factor EOMES at rest (see, e.g., FIG. 7J). Upon antigenic activation and expansion, clone 1 now produced cells that expressed either IL13, EOMES, or both; clone 3 produced cells that expressed IL13 or EOMES in a mutually exclusive manner; clone 2 increased the frequency of expression of EOMES but never IL13 (see, e.g., FIG. 7J). Consistently, upon expansion, pp65₄₉₅₋₅₀₃-specific T cell clones exhibited further clonal diversity in the expression of just two Th2 cytokines IL4 and IL13 (see, e.g., FIG. 7T). Thus, each TCR may recognize the same antigen differently to drive diverse transcriptional programs and cell states.

Together, ENTER-seq enabled a systematic dissection of T cell specificities, resting cell states, and antigen-evoked cell fate potentials after a viral infection in patients. Anti-CMV T cells transition in cytotoxicity and type-I IFN response from TEMRA T cells to TEM T cells, accompanied by upregulation of Th2 cytokine genes in specific T cell clones after peptide induced antigen-specific expansion (see, e.g., FIG. 7K).

### Example 11

### General Materials and Methods for Examples 1-10

### Plasmid Cloning and Construction.

Primers were ordered from IDT DNA technologies, and gene fragments were synthesized by twist bioscience and IDT. Table 3 shows the list of vector designs used in this study. All the constructs were made by Gibson assembly (New England Biolabs) in general. Briefly pMD2.G (addgene #12259) was digested with EcoRI to remove wild-type VSV-g gene fragment. It assembled with mutated VSV-g (K37Q and R354Q introduced by PCR primers to generate the VSV-g double mutant. psPax2 (addgene #12260) was digested with BsiWI and SphI to fuse eGFP after MA. To generate packaging vector with NC-eGFP/NC-mNeon fusion, psPAX2-D64V-NC-MS2 (addgene #122944) was digested with SphI and BspEI sequentially. Then part of gag and eGFP or mNeon were assembled together with backbone. GFP-VPR is obtained from Addgene (#83374).

To generate HPV16_L2 antigen specific BCR, light chain and heavy chain were amplified separately from vector JWW-1 (addgene #66748) and connected by a 2A peptide. Then it was inserted into a piggybac vector after CMV promoter (PB-CMV), after which, a PDGFR transmembrane (TM) domain and 2A-mCherry were added to express this antibody on cell surface. Anti-Her2 BCR was cloned in the same way from the source Trastuzumab vector (addgene #61883). To generate anti-SAR2-RBD BCR, DNA fragments encoding the light and heavy chain of a RBD antibody (Protein Data Bank under accession number 7BWJ, (Ju et al., 2020)) were codon optimized and synthesized (Twist Bio). Afterward, a signal peptide was added to each chain and heavy chain was further extended to full length with a human IgG1 Fc and a PDGFR TM sequence. The BCR was then inserted into a lentiviral vector driven by a SFFV promoter with hygromycin resistance.

Single chain format of NY-ESO-1 TCR (Roth et al., Nature 2018; Clone 1G4, wild-type (1G4 wt) and its mutated version (a95:LY) with high affinity, alpha and beta chain in tandem linked by a 2A peptide (Robbins et al., 2008) was synthesized and inserted into a lentiviral vector with hygromycin resistance. TCR5, which binds to a p5 peptide from CMV virus was amplified from alpha (addgene #164999) and beta chain (addgene #165000) and made into single chain form as with NY-ESO-1 TCR above.

For displaying antigen and HLA peptide complex on viral surface, first a cloning lentiviral vector was generated with a strong CMV promoter, multiple cloning cites and the WPRE element to enhance the expression. CD19-CAR vector was generated by inserting a scFv CD19 (kindly provided by Mackall lab) with a CD8 stalking linker and TM into the lentiviral plasmid followed by 2A-puromycin and 2A-eGFP. scFv CD19 and TMs were replaced to generate other antigen candidates including HPV-L2 antigen, CD40L (addgene #125795) and CD40L mutant (addgene #125796). For TM domain screening, the TM was swapped with 10 alternatives in the HPV-L2 antigen viral vector (Table 4). DNA fragment of SAR2 spike RBD domain was synthesized and inserted the lentiviral expression vector followed by CD8 stalking linker and TM domain similar to the above description. For Her2 display, truncated Her2 (a1-a700) fragment including its native TM and additional 55-aa cytoplasmic tail was amplified from WT HER2 (addgene #16257), and inserted into the above vector.

To display pMHC complex, a single chain vector was built, which has a signal peptide, antigen peptide, a G4S linker, beta2 microglobulin (B2M), a second G4S linker and HLA allele in tandem. DNA that encodes human growth hormone signal peptide to beta2 microglobulin was synthesized and inserted into lentiviral vector together with HLA allele. Here HLA allele A0201 was amplified from addgene vector #119052, and allele A0101 was from addgene #165009. Two cysteine mutations were introduced to stabilize the peptide binding by a bisulfide bond between Y84C of HLA allele and G2C that lies in the G4S linker after peptide. To adapt it to 10× Genomics sequencing platform, a 10×TSO sequence (Table 6) was further inserted in the linker between B2M and HLA encoding amino acids SHIRN and a 10×PCR handle in 5′UTR after CMV promoter (Table 6). A cloning vector was built by replacing antigen peptide with 2 esp3I sites, where various HLA peptides (Table 5) can be suitably inserted.

Various vectors were generated for delivery purpose. The VSV-G in pMD vector was first replaced with different envelope proteins such as RBD, HER2, pp65-HLA-A2 in the same approach as VSV-g mutant. Next, cargo delivery vectors were constructed where cargos such as HSV-TK-2A-egfp (HSV-TK from addgene #33308), and eGFP only were driven by Ef1a promoter in a lentiviral vector. For shRNA delivery, different shRNA were placed under human U6 promoter in a lentiviral vector containing eGFP and puromycin as fluorescent and selection markers. mScarlet transgene was inserted after EF1 short promoter in a lentiviral vector with puromycin resistance for labeling cells with a red fluorescent protein.

### Transfection and Lentiviral Production

To generate regular lentivirus for cell line infection and production, per 6-well, HEK 293T were transfected with a viral expression vector (2 ug), pMD2.G (VSV-G wild type) (1 ug), and psPax2 (2 ug) with lipofectamine 3000. The media was changed one next day, and viral supernatant were collected twice at 48 hours and 72 hours respectively. The virus was concentrated with 4× Lenti-X according to manufacturer's protocol, and stored at 20× concentrated in −80° C. For making specific receptor targeting and integrating virus, VSV-G mutant was used instead. To producing antigen displaying virus that can be detected with fluorescence without integration, VSV-G mutant and fluorescent protein fused version (NC-eGFP or NC-mNeon) of psPAX2-D64V (D64V mutation on integrase) vectors were mixed with antigen expressing vector according to above ratio to transfect the HEK 293T cells. For pMHC displayed viruses, HLA-KO HEK 293T cells were used for transfection. Viruses were collected, concentrated to 40×, and stored in −80° C. To generate lentivirus for cargo delivery, per 6-well, HEK 293T were transfected with a cargo expression vector (1.6 μg), pMD2.G VSV-g mut (0.8 μg), psPax2 (1.6 μg), and envelope plasmid (1 μg) with lipofectamine 3000. Virus was collected as described above, concentrated to 40×, and stored in −80° C. before use. Lentiviral titer was determined by Lenti-X GoStix Plus kit (Takarabio) according to the manufacture's protocol.

### Cell Culture and Cell Line Production

Raji, Ramos, and Jurkat related cell lines were cultured in RPMI supplemented with 10% FBS (Invitrogen) and 1× pen/strep. HEK 293T related cells were maintained in DMEM supplemented with 10% FBS and 1× pen/strep. HLA-KO HEK 293T cells were generated by electroporation of Cas9 RNP targeting HLA-A, HLA-B, and HLA-C alleles and further sorted HLA-KO cells based on surface expression of HLA-A/B/C. Ramos cells were obtained. Jurkat TCR negative −76 cells and Jurkat expressing CMV pp65 TCR, and flu-ml TCR were obtained. To generate stable cell lines including BCR and TCR expressing cells, Ramos or Jurkat cells were infected with viruses, and selected by sorting or using drug after 4-5 days. NYESO-TCR Jurkat and RBD-BCR Ramos cells were infected with red mScarlet virus and selected with puromycin to generate mScarlet red fluorescent labeled cell lines.

### Lentiviral Infection and Viral Incubation Assay

For FIG. 1B, 30 uL concentrated lentiviruses were added into 250K Raji or Jurkat cells in 12 well plate. 3 days later, GFP signal was measured by flow cytometry. For FIGURES after FIG. 1B, 200K target cells were collected in tubes and the supernatant was removed after centrifugation. The cell pellet was resuspended in 30 uL concentrated GFP fused lentiviruses and incubated at 37° C. After 2 hr incubation, cells were stained with flow cytometry antibodies for 10 min at 4° C. (if needed), washed by RPMI medium for twice, and finally subjected to flow cytometry. To quantify binding of ENTER viruses displaying pMHC antigen variants with different TCR affinity in FIG. 2D, the viral titer was normalized and incubated 100K NY-ESO-1 TCR T cells (1G4 wild-type or a95:LY mutant) or off-target CMV-pp65 TCR+ T cells with a titration (4 ng, 20 ng, 40 ng p24 level) of ENTER viruses displaying antigen variants. Upon 2-hour incubation, cells were washed and subjected to flow cytometry to quantify GFP+ cells. To compare the sensitivity of pMHC displayed ENTER viruses and pMHC tetramers per molar basis of each reagent in FIG. 2C, 100K NY-ESO-1 TCR+ T cells were incubated with 2×10⁸ ENTER viruses (20 ng p24) displaying NY-ESO-1₁₅₇₋₁₆₅ antigen or a range of NY-ESO-1₁₅₇₋₁₆₅ pMHC tetramers (2×10⁸ to 8×10⁹) for 2 hours. The calculation of molar number for each reagent is shown below. Since 10⁴ viral particles contain 1 μg p24 protein, viruses with 20 ng p24=20*1000*10⁴=2×10⁸ viral particles. For pMHC tetramer, the molecular weight was around 500 KD (PE-streptavidin: 300 KD, pMHC tetramer:˜200 KD (50 KD monomer*4)). Thus, 1 μg pMHC tetramer=1 μg*10⁶*(6.02×10²³)/500/1000=1.2×10¹² tetramers.

### Viral Binding and Fusion Assay

20 μL CD19-scFv displayed GFP viruses were incubated with 200K Raji B cells at 4° C. or 37° C. for 2 hours. Cells were washed twice and subjected to 0.5 mg/mL proteinase K treatment for 15 min at 37° C. which will digest cell surface binding viruses. Cells prior and post proteinase K treatment were subjected to flow cytometry to quantify the percentage of GFP positive cells.

### Immunocapture Assay

10 μL Protein G Dynabeads were incubated in 1 mL blocking buffer (PBS with 0.1% BSA) for 20 min at room temperature. 2 μg anti-CD40L antibody (Cat #157009, Biolegend) or anti-VSV-G antibody (clone 8G5F11, Millipore sigma), or IgG antibody were added into beads with 100 μL blocking buffer and rotated for 30 min at 4° C. The antibody conjugated beads were washed three times and the supernatant was removed. 30 μL CD40L displayed viruses were added into beads with 30 μL blocking buffer and rotated for 1 hour at room temperature. 5 μL CD40L displayed virus from the same batch was prepared as input samples. The beads were washed three times and the supernatant was removed. 100 μL Trizol was added into beads or input sample and subjected to RNA extraction by Zymo Quick-RNA Miniprep Kit. RT-qPCR was performed using Stratagene Brilliant II SYBR Green QRT-PCR Master Mix (Agilent).

### Lentivirus Targeted Cargo Delivery

Cells were incubated with virus in media with 6 μg/ml of polybrene as described above. Delivery efficiency and specificity were assessed after 3 days with flow-cytometry (Attune NxT). When needed, the cells were first stained with PeCy7 anti-human IgG (for B-cells, clone G18-145, BD bioscience), or APC anti-human CD3 (for T-cells, clone HIT3a, Biolegend) before flow cytometry analysis. For HSV-TK cell killing assay, two population of cells with one labeled by mScarlet (off-target) and one non-fluorescent (on-target) was mixed at 1:1 ratio and incubated with virus. After 3 days, ganciclovir (GCV, Invivogen) was added to a final concentration of 0.1p g/ml, which was counted as day 0. Cell media and drug were refreshed every 3 days. After 2 days, 300 μL of cell culture were taken every day, stained with IgG or CD3 before analyzed by flow cytometry. The ratio of live on-target cells over off-target cells were calculated and plotted over the days (normalized to Day 0). Alternatively, raw count of live cells for targeted or NT population at day 4 of treatment were also compared between TK and eGFP only delivery.

For apoptosis assay with FAS shRNA delivery, Jurkat T cells were infected different shRNAs, stained with PE-FAS/CD95 (Biolegend) to compare the effect of shRNA knock-down. Mixture of CMV-Jurkat (on-target) and mScarlet+ NY-ESO-Jurkat (off-target) were incubated with shRNA virus. After 5 days, anti-FAS antibody (Clone CHI 1, Millipore Sigma) was added at 0.25 μg/ml to induce apoptosis. The cells were collected after 14 hours, and stained with APC anti-Annexin V (Biolegend) and 7-AAD according to manufacturer's protocol. Then the samples were analyzed with a flow cytometry (BD LSR II), and first gated on 7-AAD-low and Annexin V-low population. Then the ratio of transduced on-target cells over off-targeted cells were compared between FAS shRNA and control shRNA to generate a bar graph with normalization.

Lentivirus Incubation with Mixed Cell Population

For T cell mixing experiment, Flu-TCR expressing Jurkat T cells were labeled by CellTrace Violet dye (#C34571, Thermo Fisher) according to manufacturer's protocol. Violet labeled Flu-ml TCR+ T cells were mixed with NY-ESO-1-TCR+ T cells at diverse ratios including 1:1, 1:10, 1:100, 1:1000. The mixed T cells were incubated with 40 μL concentrated HLA-A2-Flu antigen displayed GFP viruses for 2 hours at 37° C. T cells were stained with CD3-APC (clone HIT3a, BioLegend) antibody, washed twice, and subjected to flow cytometry. For B cell mixing experiment, HPV-BCR expressing Ramos B cells were labeled by CellTrace Violet dye and mixed with HPV-L2 BCR expressing Ramos B cells at diverse ratios including 1:1, 1:10, 1:100, 1:1000. The mixed cells were incubated with 40 uL concentrated RBD-antigen displayed GFP viruses for 2 hours at 37° C. B cells were stained with IgG-PE-Cy7 antibody (clone G18-145, BD Biosciences), washed twice, and subjected to flow cytometry. The metrics were calculated below:
Sensitivity=percentage of GFP+ on-target cells among total on-target cells
Specificity=1−(percentage of GFP+ off-target cells among total off-target cells)
Signal-to-noise ratio=(percentage of GFP+ on-target cells among total on-target cells)/(percentage of GFP+ off-target cells among total off-target cells).

### Human Primary Immune Cell Isolation and Activation

Buffy coats from healthy donors were obtained from Stanford Blood Center with consent forms. Peripheral blood mononuclear cells (PBMC) were isolated using Lymphoprep (Cat #07811, STEMCELL Technologies) density-gradient centrifugation and cryopreserved and stored in −80° C. B cells were purified from thawed PBMCs by negative selection using EasySep Human B Cell Enrichment Kit (Cat #19844, STEMCELL Technologies) according to the manufacturer's protocol. Isolated B cells were cultured in IMDM medium supplemented with 10% FBS and 55 mM beta-mercaptoethanol at 1×10⁶ cell/mL and activated by CellXVivo Human B cell expander (1:250 dilution, R&D system) and 50 ng/mL IL2 (Cat #200-02-10 ug, PeproTech) for two days. LRS chambers from HLA-A2+ donors with CMV infections (CMV seropositive) were obtained from Stanford Blood Center with consent forms. PBMCs were isolated and stored as above. CD8+ T cells were purified from thawed PBMCs by negative selection using EasySep Human CD8+ T Cell Enrichment Kit (Cat #19053, STEMCELL Technologies) according to the manufacturer's protocol.

### Peptide Enrichment of Antigen-Specific T Cells

Short 9-mer peptides encoding CMV epitopes (compatible to HLA-A2 allele, Table S3) were synthesized by Elimbio in lyophilized powders. The peptides were dissolved in DMSO in 10 mg/mL. PBMC were isolated from donor blood described as above. PBMC were cultured in T cell medium (RPMI medium supplemented with 10% FBS, 1× penstrep, 100 mM HEPES, 55 mM beta-mercaptoethanol). Individual peptide (10 ug/mL) or pooled peptides (1 ug/mL for each peptide) were added into PBMC for culturing 10 days in T cell medium. 50 ng/mL IL-2 were added every two days. After peptide enrichment, PBMCs were incubated with viruses and/or PE-tetramer and then analyzed by flow cytometry.

### Flow Cytometry

For FIG. 1D, B cells were incubated with viruses for 2 hours and then stained with Human TruStain FcX™ (Fc Block, BioLegend), CD19-APC (clone HIB19) and CD20-V450 (clone L27) antibody in cell staining buffer (BioLegend) for 10 min at 4° C. For FIG. 2, Jurkat T cells were incubated with viruses for 2 hours and then stained with CD3-APC (clone HIT3a) and PE labeled tetramer loaded with peptides (NIH tetramer core) for 30 min at 4° C. For FIG. 5, cells were incubated with viruses for 2 hours and then stained with human Fc Block, CD3-APC, CD8-BV711 (clone SK1), tetramer-PE if needed, and viability dye for 30 min at 4° C. After staining, cells were washed twice by cell straining buffer and analyzed by flow cytometry (Attune, Thermo Fisher). All antibodies are from BioLegend if not specified. Tetramers were from NIH tetramer core.

### RNA-Seq Experiment and Analysis

CMV pp65-TCR+ T cells were incubated with 30 uL pp65₄₉₅₋₅₀₃ displayed ENTER viruses or 1 μg pp65₄₉₅₋₅₀₃ tetramer for 2 hours. Cells were washed twice and subject to RNA extraction. RNA was extracted using Quick-RNA Miniprep Kit with on-column Dnase digestion (Zymo Research). At least 100 ng RNA was used to prepare the RNA-seq library using TruSeq® Stranded mRNA Library Prep Kit (Cat #20020594, Illumina) for each sample following the manufacturer's instruction. The library was sequenced on an Illumina Nextseq to generate 2×150 paired-end reads. RNA-seq reads were mapped to the human genome (hg19) using STAR with default parameters(—outFilterMultimapNmax 1—alignEndsType EndToEnd—outSAMattributes NH HI NM MD). Quantification of aligned reads at the gene level was performed by HTseq count with default parameters(—stranded=reverse−additional-attr=gene_name). Raw counts were used to identify differentially expressed genes (DEG) using DESeq2 with size factor normalization and DEGs were identified if Benjamini & Hochberg adjusted p-value<0.01 and over 2-fold change difference of gene expression.

### ENTER-Seq Workflow of Mixed TCR-Expressed Jurkat T Cells

All primers were synthesized and ordered from IDT (Table 6). Jurkat cells expressing different TCRs were mixed together and stained a mixture of virus as described above. The GFP+ cells were sorted on BD Aria II afterward. Commercial 10× Genomics 5′ RNA kit was customized to read out HLA peptide, TCR, and transcriptome simultaneously per single cell. Immediately after sorting, the cells were washed once at 4° C. with PBS+0.4% BSA, mixed with RT (reverse transcription) mixture spiked in with customized TCRalpha RT primers (mixture of hs_TRAc_RT and NYESO_TRAc_RT) at 0.1 μM, and loaded to 10× chromium. The cDNA was amplified and cleaned up to generate the transcriptome according to manufacturer's protocol.

During cDNA cleanup, the supernatant that contains shorter fragment of HLA peptide and TCR information was further mixed with SPRISelect beads to 0.9×, and cleaned up. The library that encodes HLA peptide were generated through 2 round of nested PCRs and a final round of indexing PCR. First, the HLA peptide cDNA was enriched by 8 cycles of PCR (98° C. for 45 min, then 8× of 20 sec at 98° C. 20 sec, 20 sec at 59° C. and 30 sec at 72° C.) with 0.5 uM 10×_5pRNA_Fw, and HLA_nested_fw. After cleanup, 5 ul of elution was used the second round of PCR with 0.5 um nested primer and Illumina adapters P7_Tru_HLA_fw and P5_adapter primer as above. Last, take 5 ul of elution to generate final library with Illumina Truseq based index primers. The above primers were designed in a way compatible with dual index, thus either customized index primer or 10× dual index primers can be used here.

To read out TCR information in Jurkat cell lines, a library that covers VDJ part of TCR alpha to infer cell's TCR identity was generated. First, the TCR DNA were enriched by nested PCR (specifically, 98° C. for 45 min, then 8× of 20 sec at 98° C. 20 sec, 20 sec at 59° C. and 30 sec at 72° C.) with 0.5 μM 10×_5pRNA_Fw and 0.5 uM mix of nyeso_TRAc_rev and hs_TRAc_rev targeting two different TCR. Next, 5 ul of eluent was taken to run the second round of nested PCR with Illumina adapter (a mixture of P7_TRAc_nyeso_Rev and P7_TRAc_hs_Rev) and P5_adapter for 8 cycles, followed by a final index PCR similar to HLA libraries.

### ENTER-Seq Analysis of Mixed TCR-Expressed Jurkat T Cells

The libraries were sequenced using Illumina's Novaseq and Miseq platforms. Transcriptome fastq files were analyzed using 10×'s CellRanger to provide single cell barcodes. The fastq files of TCR libraries were mapped to TCR alpha chain with custom python script. The UMI count of each type TCR per cells' barcodes was calculated. To exclude doublet, it was set that, per gem barcode, the UMI count for the dominant TCR was at least 10 times more than those non-dominant TCR species. Next, HLA peptide reads were processed using CellRanger count with peptide sequence as feature reference. Downstream analysis and plots were generated with matplotlib package in python.

### ENTER-Seq Workflow of Primary T Cells Prior or Post CMV Antigen Peptide-Induced Expansion Ex Vivo

Peptide stimulated donor PBMC were collected and stain with a mixture of 12 viruses displaying CMV antigen epitopes including IE1₈₁₋₈₉, IE1₃₁₆₋₃₂₄, US150A₁₅₂₋₁₆₁, US8₇₄₋₈₂, UL100₂₀₀₋₂₀₈, UL46₁₀₀₋₁₀₈, pp65₄₁₇₋₄₂₅, pp65₃₂₅₋₃₃₃, pp65₁₈₈₋₁₉₆, pp65₁₂₀₋₁₂₈, pp65₄₉₅₋₅₀₃, and pp65₁₄₋₂₂. The peptide sequences for CMV antigens were listed in Table 5. After 2 hours, cells were also stained with barcoded antibodies CD45RA, CD45RO, and IL7R (Biolegend totalseq-C, cat #304163, cat #304259, cat #351356), live dead dye, CD3-APC and CD8-BV711 for 20 min on ice. Sample from each donor was also stained with unique hash-tag barcoded antibody (Biolegend totalseq-C, cat #394661, cat #394663). After two washes, CD8+ CD3+ GFP+ cells were sorted to run on 10× Genomics platform using 5′ RNA and VDJ kits according to manufacturer's protocol. Here, the following libraries were obtained per sample: 10× gene expression library, VDJ library, and feature barcoded CITE-seq library according to manufacturer's protocol. In addition, HLA peptide library was generated in the same way as described above. Final libraries were sequenced on either Illumina Miseq, Nextseq 550 or Novaseq 6000. For ENTER-seq of primary T cells isolated directly from patient blood samples without peptide stimulation/expansion, total CD8+ T cells were first purified from cryopreserved patient PBMC samples using EasySep human CD8+ T cell isolation kit (Cat #17953, STEMCELL Technologies) following the user's protocol. Next, a mixture of top 3 ENTER pMHC viruses (pp65₄₉₅₋₅₀₃, US8₇₄₋₈₂ and UL100₂₀₀₋₂₀₈) was prepared for 2 hours at 37° C. Later steps of antibody staining, flow cytometry sorting, and 10× Genomics library generation were the same as ex vivo expanded T cells described above.

ENTER-Seq Analysis of Primary Cells from CMV Seropositive Donors

The scRNA-seq reads were aligned to GRCh38 genome and quantified using CellRanger count (10× Genomics). The CITE-seq reads were processed using CellRanger count with antibody oligo barcode as feature reference. The TCR-seq reads were mapped to VDJ compatible reference (refdata-CellRanger-vdj-GRCh38-alts-ensembl-5.0.0) using CellRanger vdj (10× Genomics). HLA peptide reads were processed using CellRanger count with peptide sequence as feature reference.

The later analyses for single cell RNA-seq and CITE-seq were performed using SCANPY (Wolf et al., 2018). Cells with less than 200 genes detected or greater than 10% mitochondrial RNA reads were excluded from analysis. Doublet cells were removed using CITE-seq analysis of barcoded hashtag antibody labeling donor origins. For cell clustering, raw UMI counts were first normalized by total counts to correct library size and then log-normalized. Variable genes were called using scanpy.pp.highly_variable_genes( ) with default parameters. Variable TCR genes were removed before principal component analysis (PCA) to prevent clustering bias from variable TCR transcripts. Next, the effects of total counts per cell and the percentage of mitochondrial genes were regressed out, and then scale the data to unit variance. Scaled data were used as input into PCA analysis on the basis of variable genes (without TCR genes). Clusters were identified using Leiden graph-clustering method with the first 40 principal components. To determine if ENTER virus-induced gene expression can impact T cell state and clustering, Leiden graph-clusterings before and after removal of 28 ENTER virus induced genes were performed that are identified from bulk RNA-seq data. UMAP plots were generated using scanpy.tl.umap( ) and scanpy.pl.umap( ) with default parameters. Heatmap plots were generated using scanpy.tl.heatmap( ) with raw value or z score scaled gene expression.

Initial clusters were annotated using expression of known markers including CD3E, CD4, CD8A, CD45RA, CD45RO, CCR7, GZMB, and KLRB1. All CD8+ T cells were CD3E+CD8A+CD4−. Naïve T cells were CD45RA+CCR7+. Central memory T cells (TCM) were CD45RA-CCR7+. Effector memory T (TEM) cells were CD45RO+CCR7−. Terminal effector cells re-expressing CD45RA (TEMRA) were CD45RA+CCR7−CD45RO−, MAIT cells were KLRB1+CXCR6+ TRAV1-2+. The gene score was calculated using scanpy.tl.score_genes( ) with ctrl_size=500 and use_raw=True. The gene set of cytotoxic genes were curated from well-established cytotoxic molecules. The T cell exhaustion genes, T cell activation genes, and type-I IFN response genes were selected from previous literature (Yost et al., 2019).

TCR relevant analyses were performed using Scirpy (Sturm et al., 2020). The contig annotation files generated by CellRanger vdj were used as input for TCR analysis. TCR qualities were analyzed using scirpy.tl.chain_qc( ). The TCR clonotypes were defined using scirpy.pp.ir_dist( ) and scirpy.tl.define_clonotypes( ) with default parameters based on CDR3 nucleotide sequence similarity. The TCR clonotypes were visualized on a network using scirpy. tl.clonotype_network( ) with min_cells=3. The CDR3 amino acid compositions were generated using weblogo (Crooks et al., 2004). Cells with more than 5 raw count of HLA peptides for any individual antigens were labeled as antigen-specific T cells. The antigen peptide count per cell was quantified using log(count+1) transformation. The pp65-specific T cells were divided into diverse clonally expanded cells with clone size >50, or >10, or ≥1. The distribution of antigen peptide count bound per cell in different clonally expanded T cells were showed in a violin plot. Using dominant TCR clonotypes as a barcode, 2D density plots were generated using kdeplot( ) function to show cytotoxic gene score and type-I IFN gene score of T cells with same TCR sequence before and after antigen-induced expansion. All plots (e.g. violin plots, scatter plots, density plots and Bar plots) were generated by Python matplotlib and seaborn.

Using TCR as a barcode, false negative T cells were identified in ENTER negative population who share the same TCR sequences with the dominant antigen-specific T cells. Similarly, false positive antigen-specific T cells such as pp65-specific T cells could be generated by tracking TCR sequences between pp65-specific T cells with dominant clones and other CMV-antigen specific T cells or ENTER-negative T cells. The false negative rate (FNR) and false positive rate (FPR) were further calculated for top 3 antigen epitopes. For pp65₄₉₅₋₅₀₃-specific T cells, FNR=0.19%; FDR=0.36%. For US8₇₄₋₈₂-specific T cells, FNR=2.73%; FDR=0.18%. For UL100₂₀₀₋₂₀₈-specific T cells, FNR=0%; FDR=0.07%.

While the disclosure has been particularly shown and described with reference to specific embodiments (some of which are preferred embodiments), it should be understood by those having skill in the art that various changes in form and detail can be made therein without departing from the spirit and scope of the present disclosure as disclosed herein. There is no intention, therefore, of limitations to the exact abstract and disclosure herein presented.

TABLE 3
List of vectors constructed in these studies

| Vector | Notes |
|---|---|
| PMD2.G-VSV-G-mut | Double mutation for VSV-G |
| psPax2-MA-eGFP | EGFP fused with MA in gag |
| psPax2-NC-eGFP | EGFP fused with NC in gag |
| psPax2-NC-mNeon | mNeon fused with NC in gag |
| plenti-sffv-tcr5-2a-hygro | Expressing TCR specific to CMV_p5 A0101 |
| plenti-EF1-NYESO_TCR-2a-hygro | Expressing TCR specific for NYESO A0201 |
| PBCMV-HPV16_L2-BCR -2a- | Expressing HPV16L2 antibody on cell surface. |
| mCherry | Puro resistant |
| plentiCMV-aCD19-Puro-2A-eGFP | Display CD19 CAR domain on virus surface |
| PlentiCMV-CD40L--Puro-2A- | Display CD40L wild-type and mutant on virus |
| eGFP | |
| pLentiCMV-antigen-TM | For displaying antigen domain on virus surface |
| plentiCMV-sp-HPV-TM | For swapping transmembrane domains. |
| plentiCMV-sct-b2m-HLA-A0201 | Display HLA-peptide complex on virus |
| plentiCMV10x-sct-b2m-HLA- | Display HLA-peptide complex on virus, used with |
| A0201 | 10xGenomics library preparation |
| plentiCMV-sct-esp3i-b2m-HLA- | Cloning vector for inserting HLA_peptide |
| A0201 | |
| plentiCMV10x-sct-esp3i-b2m- | Cloning vector for inserting HLA_peptide, 10x |
| HLA-A0201 | Genomics version |
| plentiCMV-sct-p5-b2m-HLA- | Display HLA-peptide cmv_p5 complex on virus |
| A0101 | |
| plentiCMV10x-sct-p5-b2m-HLA- | 10x Genomic version of above |
| A0101 | |

TABLE 4
List of TM domain sequences

| Gene | Transmembrane domain | Cytoplasm domain |
|---|---|---|
| CD162 | LLAILILALVATIFFVCTVVL | AVRLSRKGHMYPVRN |
| (PSGL1) | (SEQ ID NO: 34) | (SEQ ID NO: 44) |
| LFA-1 | YLYVLSGIGGLLLLLLIFIVL | YKVGFFKRNLK |
| | (SEQ ID NO: 35) | (SEQ ID NO: 45) |
| CD62L | PLFIPVAVMVTAFSGLAFIIW | RRLKKGKKSKR |
| | LA (SEQ ID NO: 36) | (SEQ ID NO: 46) |
| CD49d | IVIISSSLLLGLIVLLLISYV | AGFFKRQYKS |
| | MWK (SEQ ID NO: 37) | (SEQ ID NO: 47) |
| CD43 | GMLPVAVLVALLAVIVLVALL | WRRRQKRRT |
| | LL (SEQ ID NO: 38) | (SEQ ID NO: 48) |
| HLA-A2 | IVGIIAGLVLFGAVITGAVVA | RRKSSDRKG |
| | AVMW (SEQ ID NO: 39) | (SEQ ID NO: 49) |
| VSV-G | FFFIIGLIIGLFLVLRVGIHL | KLKHTKKRQI |
| | CI (SEQ ID NO: 40) | (SEQ ID NO: 50) |
| ICAM1 | IVIITVVAAAVIMGTAGLSTY | NRQRKIKKYRL |
| | LY (SEQ ID NO: 41) | (SEQ ID NO: 51) |
| HLA- | NVVCALGLTVGLVGIIIGTIF | KGLRKSNAAERRGPL |
| Dra1 | II (SEQ ID NO: 42) | (SEQ ID NO: 52) |
| PDGFR | GTVVVISAILALVVLTIISLI | WQKKPR |
| | ILIML (SEQ ID NO: 43) | (SEQ ID NO: 53) |

TABLE 5
List of HLA peptide sequence:

| | | HLA | |
|---|---|---|---|
| Name | Peptide | allele | Antigen |
| NY-ESO-1₁₅₇₋₁₆₅ | SLLMWITQC (SEQ ID NO: 1) | A0201 | NY-ESO-1 (157-165) |
| NY-ESO-1₁₅₇₋₁₆₅ | SLAMWITQC (SEQ ID NO: 2) | A0201 | NY-ESO-1 mutant |
| L3A | | | peptide |
| NY-ESO-1₁₅₇₋₁₆₅ | SLLMWIAQC (SEQ ID NO: 3) | A0201 | NY-ESO-1 mutant |
| T7A | | | peptide |
| pp65₄₉₅₋₅₀₃ | NLVPMVATV (SEQ ID NO: 4) | A0201 | CMV pp65 (495-503) |
| p65₃₆₃₋₃₇₃ | YSEHPTFTSQY (SEQ ID NO: 5) | A0101 | CMV pp65 (363-373) |
| M1₅₈₋₆₆ | GILGFVFTL (SEQ ID NO: 6) | A0201 | Influenza M1 (58-66) |
| IE1₃₁₆₋₃₂₄ | VLEETSVML (SEQ ID NO: 7) | A0201 | CMV IE1 (316-324) |
| IE1₈₁₋₈₉ | VLAELVKQI (SEQ ID NO: 8) | A0201 | CMV IE1 (81-89) |
| pp65₁₄₋₂₂ | VLGPISGHV (SEQ ID NO: 9) | A0201 | CMV pp65 (14-22) |
| pp65₁₂₀₋₁₂₈ | MLNIPSINV (SEQ ID NO: 10) | A0201 | CMV pp65 (120-128) |
| pp65₁₈₈₋₁₉₆ | FPTKDVALR (SEQ ID NO: 11) | A0201 | CMV pp65 (188-196) |
| pp65₃₂₅₋₃₃₃ | QIFLEVQAI (SEQ ID NO: 12) | A0201 | CMV pp65 (325-333) |
| pp65₄₁₇₋₄₂₅ | TPRVTGGGA (SEQ ID NO: 13) | A0201 | CMV pp65 (417-425) |
| UL46₁₀₀₋₁₀₈ | CLLESVYTA (SEQ ID NO: 14) | A0201 | CMV UL46 (100-108) |
| UL100₂₀₀₋₂₀₈ | TLIVNLVEV (SEQ ID NO: 15) | A0201 | CMV UL100 (200-208) |
| US8₇₄₋₈₂ | GVLDAVWRV (SEQ ID NO: 16) | A0201 | CMV US8 (74-82) |
| US150A₁₅₂₋₁₆₁ | ALWDVALLEV (SEQ ID NO: 17) | A0201 | CMV US150A (152-161) |
| pp65₁₂₀₋₁₂₈ | MLNIPSINV (SEQ ID NO: 18) | A0201 | CMV pp65 (120-128) |

TABLE 6
DNA Oligo sequences

| | |
|---|---|
| hs_TRAc_RT | gttgaaggcgtttgca (SEQ ID NO: 19) |
| nyeso_TRAc_RT | TCTTCTCAACGAGTTTAACGT (SEQ ID NO: 20) |
| HLA_nested_fw | ccaccatggcgacgggttca (SEQ ID NO: 21) |
| 10x_5pRNA_Fw | ACACTCTTTCCctacacgacgctcttccgatct (SEQ ID NO: |
| | 22) |
| P7_Tru_HLA_fw | GTGACTGGAGTTCAGACGTGTGCTCTTCCGATCTggttacaggagggc |
| | tcggca (SEQ ID NO: 23) |
| P5_adapter | ACACTCTTTCCCTACACGACGCTCT (SEQ ID NO: 24) |
| nyeso_TRAc_rev | AACGTCACAGGAGCTTTCGGGA (SEQ ID NO: 25) |
| hs_TRAc_rev | TGAAGGCGTTTGCACATGCA (SEQ ID NO: 26) |
| P7_TRAc_nyeso_Rev | GTGACTGGAGTTCAGACGTGTGCTCTTCCGATCTgggttttggatgta |
| | cggatgaac (SEQ ID NO: 27) |
| P7_TRAc_hs_Rev | GTGACTGGAGTTCAGACGTGTGCTCTTCCGATCTcagctggtacacgg |
| | cagg (SEQ ID NO: 28) |
| 10xTSO_oligo | tCCCATATAAGAAAc (SEQ ID NO: 29) |
| 10xPCR_handle | AAGCAGTGGTATCAACGCAGAGTAC (SEQ ID NO: 30) |
| Fas shRNA-1 | CCCTTGTGTTTGGAATTATAACTCGAGTTATAATTCCAAACACAAGGG |
| sequence under U6 | (SEQ ID NO: 31) |
| Fas shRNA-2 | GCGTATGACACATTGATTAAACTCGAGTTTAATCAATGTGTCATACGC |
| sequence under U6 | (SEQ ID NO: 32) |
| Fas shRNA-3 | CCTGAAACAGTGGCAATAAATCTCGAGATTTATTGCCACTGTTTCAGG |
| sequence under U6 | (SEQ ID NO: 33) |

### REFERENCES

1. Appay, V., Dunbar, P. R., Callan, M., Klenerman, P., Gillespie, G. M. A., Papagno, L., Ogg, G. S., King, A., Lechner, F., Spina, C. A., et al. (2002). Memory CD8+ T cells vary in differentiation phenotype in different persistent virus infections. Nat Med 8, 379-385. doi.org/10.1038/nm0402-379.
2. Armingol, E., Officer, A., Harismendy, O., and Lewis, N. E. (2021). Deciphering cell-cell interactions and communication from gene expression. Nat Rev Genet 22, 71-88. doi.org/10.1038/s41576-020-00292-x.
3. Bai, Z., Woodhouse, S., Zhao, Z., Arya, R., Govek, K., Kim, D., Lundh, S., Baysoy, A., Sun, H., Deng, Y., et al. (2022). Single-cell antigen-specific landscape of CAR T infusion product identifies determinants of CD19-positive relapse in patients with ALL. Sci Adv 8, eabj2820. doi.org/10.1126/sciadv.abj2820.
4. Beltinger, C., Fulda, S., Kammertoens, T., Meyer, E., Uckert, W., and Debatin, K. M. (1999). Herpes simplex virus thymidine kinase/ganciclovir-induced apoptosis involves ligand-independent death receptor aggregation and activation of caspases. Proceedings of the National Academy of Sciences 96, 8699-8704. doi.org/10.1073/pnas.96.15.8699.
5. Bentzen, A. K., and Hadrup, S. R. (2017). Evolution of MHC-based technologies used for detection of antigen-responsive T cells. Cancer Immunol Immunother 66, 657-666. doi.org/10.1007/s00262-017-1971-5.
6. Bentzen, A. K., Marquard, A. M., Lyngaa, R., Saini, S. K., Ramskov, S., Donia, M., Such, L., Furness, A. J. S., McGranahan, N., Rosenthal, R., et al. (2016). Large-scale detection of antigen-specific T cells using peptide-MHC-I multimers labeled with DNA barcodes. Nat Biotechnol 34, 1037-1045. doi.org/10.1038/nbt.3662.
7. Billerbeck, E., Blum, H. E., and Thimme, R. (2007). Parallel expansion of human virus-specific FoxP3− effector memory and de novo-generated FoxP3+ regulatory CD8+ T cells upon antigen recognition in vitro. J Immunol 179, 1039-1048. doi.org/10.4049/jimmunol.179.2.1039.
8. Birnbaum, M. E., Dong, S., and Garcia, K. C. (2012). Diversity-oriented approaches for interrogating T-cell receptor repertoire, ligand recognition, and function. Immunol Rev 250, 82-101. doi.org/10.1111/imr.12006.
9. Bollard, C. M., Kuehnle, I., Leen, A., Rooney, C. M., and Heslop, H. E. (2004). Adoptive immunotherapy for posttransplantation viral infections. Biol Blood Marrow Transplant 10, 143-155. doi.org/10.1016/j.bbmt.2003.09.017.
10. Briggs, J. A. G., Simon, M. N., Gross, I., Krausslich, H. G., Fuller, S. D., Vogt, V. M., and Johnson, M. C. (2004). The stoichiometry of Gag protein in HIV-1. Nat Struct Mol Biol 11, 672-675. doi.org/10.1038/nsmb785.
11. Briney, B., Inderbitzin, A., Joyce, C., and Burton, D. R. (2019). Commonality despite exceptional diversity in the baseline human antibody repertoire. Nature 566, 393-397. doi.org/10.1038/s41586-019-0879-y.
12. Buchholz, C. J., Mühlebach, M. D., and Cichutek, K. (2009). Lentiviral vectors with measles virus glycoproteins—dream team for gene transfer? Trends Biotechnol 27, 259-265. doi.org/10.1016/j.tibtech.2009.02.002.
13. Buchholz, C. J., Friedel, T., and Büning, H. (2015). Surface-Engineered Viral Vectors for Selective and Cell Type-Specific Gene Delivery. Trends Biotechnol 33, 777-790. doi.org/10.1016/j.tibtech.2015.09.008.
14. Buenrostro, J. D., Wu, B., Litzenburger, U. M., Ruff, D., Gonzales, M. L., Snyder, M. P., Chang, H. Y., and Greenleaf, W. J. (2015). Single-cell chromatin accessibility reveals principles of regulatory variation. Nature 523, 486-490. doi.org/10.1038/nature14590.
15. Burbelo, P. D., Iadarola, M. J., Keller, J. M., and Warner, B. M. (2021). Autoantibodies Targeting Intracellular and Extracellular Proteins in Autoimmunity. Front Immunol 12, 548469. doi.org/10.3389/fimmu.2021.548469.
16. Burnie, J., and Guzzo, C. (2019). The Incorporation of Host Proteins into the External HIV-1 Envelope. Viruses 11, E85. doi.org/10.3390/v11010085.
17. Burnie, J., Tang, V. A., Welsh, J. A., Persaud, A. T., Thaya, L., Jones, J. C., and Guzzo, C. (2020). Flow Virometry Quantification of Host Proteins on the Surface of HIV-1 Pseudovirus Particles. Viruses 12, E1296. doi.org/10.3390/v12111296.
18. Burns, J. C., Friedmann, T., Driever, W., Burrascano, M., and Yee, J. K. (1993). Vesicular stomatitis virus G glycoprotein pseudotyped retroviral vectors: concentration to very high titer and efficient gene transfer into mammalian and nonmammalian cells. Proc Natl Acad Sci USA 90, 8033-8037. doi.org/10.1073/pnas.90.17.8033.
19. Cantin, R., Fortin, J. F., and Tremblay, M. (1996). The amount of host HLA-DR proteins acquired by HIV-1 is virus strain- and cell type-specific. Virology 218, 372-381. doi.org/10.1006/viro.1996.0206.
20. Certo, M. T., Ryu, B. Y., Annis, J. E., Garibov, M., Jarjour, J., Rawlings, D. J., and Scharenberg, A. M. (2011). Tracking genome engineering outcome at individual DNA breakpoints. Nat Methods 8, 671-676. doi.org/10.1038/nmeth.1648.
21. Chandran, S. S., and Klebanoff, C. A. (2019). T cell receptor-based cancer immunotherapy: Emerging efficacy and pathways of resistance. Immunol Rev 290, 127-147. doi.org/10.1111/imr.12772.
22. Chertova, E., Chertov, O., Coren, L. V., Roser, J. D., Trubey, C. M., Bess, J. W., Sowder, R. C., Barsov, E., Hood, B. L., Fisher, R. J., et al. (2006). Proteomic and biochemical analysis of purified human immunodeficiency virus type 1 produced from infected monocyte-derived macrophages. J Virol 80, 9039-9052. doi.org/10.1128/JVI.01013-06.
23. Churlaud, G., Pitoiset, F., Jebbawi, F., Lorenzon, R., Bellier, B., Rosenzwajg, M., and Klatzmann, D. (2015). Human and Mouse CD8(+)CD25(+)FOXP3(+) Regulatory T Cells at Steady State and during Interleukin-2 Therapy. Front Immunol 6, 171. doi.org/10.3389/fimmu.2015.00171.
24. Crooks, G. E., Hon, G., Chandonia, J. M., and Brenner, S. E. (2004). WebLogo: a sequence logo generator. Genome Res 14, 1188-1190. doi.org/10.1101/gr.849004.
25. Davis, M. M., and Bjorkman, P. J. (1988). T-cell antigen receptor genes and T-cell recognition. Nature 334, 395-402. doi.org/10.1038/334395a0.
26. Davis, M. M., and Boyd, S. D. (2019). Recent progress in the analysis of αβT cell and B cell receptor repertoires. Curr Opin Immunol 59, 109-114. doi.org/10.1016/j.coi.2019.05.012.
27. De Guzman, R. N., Wu, Z. R., Stalling, C. C., Pappalardo, L., Borer, P. N., and Summers, M. F. (1998). Structure of the HIV-1 nucleocapsid protein bound to the SL3 psi-RNA recognition element. Science 279, 384-388. doi.org/10.1126/science.279.5349.384.
28. Derhovanessian, E., Maier, A. B., Hahnel, K., Beck, R., de Craen, A. J. M., Slagboom, E. P., Westendorp, R. G. J., and Pawelec, G. (2011). Infection with cytomegalovirus but not herpes simplex virus induces the accumulation of late-differentiated CD4+ and CD8+ T-cells in humans. J Gen Virol 92, 2746-2756. doi.org/10.1099/vir.0.036004-0.
29. Dobson, C. S., Reich, A. N., Gaglione, S., Smith, B. E., Kim, E. J., Dong, J., Ronsard, L., Okonkwo, V., Lingwood, D., Dougan, M., et al. (2022). Antigen identification and high-throughput interaction mapping by reprogramming viral entry. Nat Methods 19, 449-460. doi.org/10.1038/s41592-022-01436-z.
30. Dupic, T., Bensouda Koraichi, M., Minervina, A. A., Pogorelyy, M. V., Mora, T., and Walczak, A. M. (2021). Immune fingerprinting through repertoire similarity. PLoS Genet 17, e1009301. doi.org/10.1371/joumal.pgen.1009301.
31. Einsele, H., Kapp, M., and Grigoleit, G. U. (2008). CMV-specific T cell therapy. Blood Cells Mol Dis 40, 71-75. doi.org/10.1016/j.bcmd.2007.07.002.
32. Elkington, R., Walker, S., Crough, T., Menzies, M., Tellam, J., Bharadwaj, M., and Khanna, R. (2003). Ex vivo profiling of CD8+-T-cell responses to human cytomegalovirus reveals broad and multispecific reactivities in healthy virus carriers. J Virol 77, 5226-5240. doi.org/10.1128/jvi.77.9.5226-5240.2003.
33. Finkelshtein, D., Werman, A., Novick, D., Barak, S., and Rubinstein, M. (2013). LDL receptor and its family members serve as the cellular receptors for vesicular stomatitis virus. Proc Natl Acad Sci USA 110, 7306-7311. doi.org/10.1073/pnas.1214441110.
34. Fontenot, J. D., Gavin, M. A., and Rudensky, A. Y. (2003). Foxp3 programs the development and function of CD4+CD25+ regulatory T cells. Nat Immunol 4, 330-336. doi.org/10.1038/ni904.
35. Garboczi, D. N., Hung, D. T., and Wiley, D. C. (1992). HLA-A2-peptide complexes: refolding and crystallization of molecules expressed in Escherichia coli and complexed with single antigenic peptides. Proc Natl Acad Sci USA 89, 3429-3433. doi.org/10.1073/pnas.89.8.3429.
36. Gerdemann, U., Katari, U. L., Papadopoulou, A., Keirnan, J. M., Craddock, J. A., Liu, H., Martinez, C. A., Kennedy-Nasser, A., Leung, K. S., Gottschalk, S. M., et al. (2013). Safety and clinical efficacy of rapidly-generated trivirus-directed T cells as treatment for adenovirus, EBV, and CMV infections after allogeneic hematopoietic stem cell transplant.

Mol Ther 21, 2113-2121. doi.org/10.1038/mt.2013.151.
37. Gillespie, G. M., Wills, M. R., Appay, V., O'Callaghan, C., Murphy, M., Smith, N., Sissons, P., Rowland-Jones, S., Bell, J. I., and Moss, P. A. (2000). Functional heterogeneity and high frequencies of cytomegalovirus-specific CD8(+) T lymphocytes in healthy seropositive donors. J Virol 74, 8140-8150. doi.org/10.1128/jvi.74.17.8140-8150.2000.
38. Gotch, F., Rothbard, J., Howland, K., Townsend, A., and McMichael, A. (1987). Cytotoxic T lymphocytes recognize a fragment of influenza virus matrix protein in association with HLA-A2. Nature 326, 881-882. doi.org/10.1038/326881a0.
39. Grover, J. R., Veatch, S. L., and Ono, A. (2015). Basic motifs target PSGL-1, CD43, and CD44 to plasma membrane sites where HIV-1 assembles. J Virol 89, 454-467. doi.org/10.1128/JVI.02178-14.
40. Guo, X. Z. J., and Elledge, S. J. (2022). V-CARMA: A tool for the detection and modification of antigen-specific T cells. Proc Natl Acad Sci USA 119, e2116277119. doi.org/10.1073/pnas.2116277119.
41. Gutierrez, C., and Schiff, R. (2011). HER2: biology, detection, and clinical implications. Arch Pathol Lab Med 135, 55-62. doi.org/10.5858/2010-0454-RAR.1.
42. Hinrichs, C. S., and Rosenberg, S. A. (2014). Exploiting the curative potential of adoptive T-cell therapy for cancer. Immunol Rev 257, 56-71. doi.org/10.1111/imr.12132.
43. Hu, Z., Ott, P. A., and Wu, C. J. (2018). Towards personalized, tumour-specific, therapeutic vaccines for cancer. Nat Rev Immunol 18, 168-182. doi.org/10.1038/nri.2017.131.
44. Jager, E., Chen, Y. T., Drijfhout, J. W., Karbach, J., Ringhoffer, M., Jager, D., Arand, M., Wada, H., Noguchi, Y., Stockert, E., et al. (1998). Simultaneous humoral and cellular immune response against cancer-testis antigen NY-ESO-1: definition of human histocompatibility leukocyte antigen (HLA)-A2-binding peptide epitopes. J Exp Med 187, 265-270. doi.org/10.1084/jem.187.2.265.
45. Jalaguier, P., Cantin, R., Maaroufi, H., and Tremblay, M. J. (2015). Selective acquisition of host-derived ICAM-1 by HIV-1 is a matrix-dependent process. J Virol 89, 323-336. doi.org/10.1128/JVI.02701-14.
46. Joglekar, A. V., Leonard, M. T., Jeppson, J. D., Swift, M., Li, G., Wong, S., Peng, S., Zaretsky, J. M., Heath, J. R., Ribas, A., et al. (2019). T cell antigen discovery via signaling and antigen-presenting bifunctional receptors. Nat Methods 16, 191-198. doi.org/10.1038/s41592-018-0304-8.
47. Ju, B., Zhang, Q., Ge, J., Wang, R., Sun, J., Ge, X., Yu, J., Shan, S., Zhou, B., Song, S., et al. (2020). Human neutralizing antibodies elicited by SARS-CoV-2 infection. Nature 584, 115-119. doi.org/10.1038/s41586-020-2380-z.
48. Kaczmarczyk, S. J., Sitaraman, K., Young, H. A., Hughes, S. H., and Chatterjee, D. K. (2011). Protein delivery using engineered virus-like particles. Proc Natl Acad Sci USA 108, 16998-17003. doi.org/10.1073/pnas.1101874108.
49. Kaya-Okur, H. S., Wu, S. J., Codomo, C. A., Pledger, E. S., Bryson, T. D., Henikoff, J. G., Ahmad, K., and Henikoff, S. (2019). CUT & Tag for efficient epigenomic profiling of small samples and single cells. Nat Commun 10, 1930. doi.org/10.1038/s41467-019-09982-5.
50. Kneissl, S., Zhou, Q., Schwenkert, M., Cosset, F. L., Verhoeyen, E., and Buchholz, C. J. (2013). CD19 and CD20 targeted vectors induce minimal activation of resting B lymphocytes. PLoS One 8, e79047. doi.org/10.1371/journal.pone.0079047.
51. Kula, T., Dezfulian, M. H., Wang, C. I., Abdelfattah, N. S., Hartman, Z. C., Wucherpfennig, K. W., Lyerly, H. K., and Elledge, S. J. (2019). T-Scan: A Genome-wide Method for the Systematic Discovery of T Cell Epitopes. Cell 178, 1016-1028.e13. doi.org/10.1016/j.cell.2019.07.009.
52. Kutluay, S. B., Zang, T., Blanco-Melo, D., Powell, C., Jannain, D., Errando, M., and Bieniasz, P. D. (2014). Global changes in the RNA binding specificity of HIV-1 gag regulate virion genesis. Cell 159, 1096-1109. doi.org/10.1016/j.cell.2014.09.057.
53. Lee, M. N., and Meyerson, M. (2021). Antigen identification for HLA class I- and HLA class II-restricted T cell receptors using cytokine-capturing antigen-presenting cells. Sci Immunol 6, eabf4001. doi.org/10.1126/sciimmunol.abf4001.
54. Lee, D. S. W., Rojas, O. L., and Gommerman, J. L. (2021). B cell depletion therapies in autoimmune disease: advances and mechanistic insights. Nat Rev Drug Discov 20, 179-199. doi.org/10.1038/s41573-020-00092-2.
55. Lehmann, A. A., Zhang, T., Reche, P. A., and Lehmann, P. V. (2020). Discordance Between the Predicted Versus the Actually Recognized CD8+ T Cell Epitopes of HCMV pp65 Antigen and Aleatory Epitope Dominance. Front Immunol 11, 618428. doi.org/10.3389/fimmu.2020.618428.
56. Li, G., Bethune, M. T., Wong, S., Joglekar, A. V., Leonard, M. T., Wang, J. K., Kim, J. T., Cheng, D., Peng, S., Zaretsky, J. M., et al. (2019). T cell antigen discovery via trogocytosis. Nat Methods 16, 183-190. doi.org/10.1038/s41592-018-0305-7.
57. Lübke, M., Spalt, S., Kowalewski, D. J., Zimmermann, C., Bauersfeld, L., Nelde, A., Bichmann, L., Marcu, A., Peper, J. K., Kohlbacher, O., et al. (2020). Identification of HCMV-derived T cell epitopes in seropositive individuals through viral deletion models. J Exp Med 217, e20191164. doi.org/10.1084/jem.20191164.
58. McCutcheon, M., Wehner, N., Wensky, A., Kushner, M., Doan, S., Hsiao, L., Calabresi, P., Ha, T., Tran, T. V., Tate, K. M., et al. (1997). A sensitive ELISPOT assay to detect low-frequency human T lymphocytes. J Immunol Methods 210, 149-166. doi.org/10.1016/s0022-1759(97)00182-8.
59. Mimitou, E. P., Cheng, A., Montalbano, A., Hao, S., Stoeckius, M., Legut, M., Roush, T., Herrera, A., Papalexi, E., Ouyang, Z., et al. (2019). Multiplexed detection of proteins, transcriptomes, clonotypes and CRISPR perturbations in single cells. Nat Methods 16, 409-412. doi.org/10.1038/s41592-019-0392-0.
60. Moore, M. D., and Hu, W. S. (2009). HIV-1 RNA dimerization: It takes two to tango. AIDS Rev 11, 91-102.
61. Nikolic, J., Belot, L., Raux, H., Legrand, P., Gaudin, Y., and A Albertini, A. (2018). Structural basis for the recognition of LDL-receptor family members by VSV glycoprotein. Nat Commun 9, 1029. doi.org/10.1038/s41467-018-03432-4.
62. Pai, J. A., and Satpathy, A. T. (2021). High-throughput and single-cell T cell receptor sequencing technologies. Nat Methods 18, 881-892. doi.org/10.1038/s41592-021-01201-8.
63. Pasqual, G., Chudnovskiy, A., Tas, J. M. J., Agudelo, M., Schweitzer, L. D., Cui, A., Hacohen, N., and Victora, G. D. (2018). Monitoring T cell-dendritic cell interactions in vivo by intercellular enzymatic labelling. Nature 553, 496-500. doi.org/10.1038/nature25442.
64. Purcell, A. W., Ramarathinam, S. H., and Ternette, N. (2019). Mass spectrometry-based identification of MHC-bound peptides for immunopeptidomics. Nat Protoc 14, 1687-1707. doi.org/10.1038/s41596-019-0133-y.
65. Ranzani, M., Cesana, D., Bartholomae, C. C., Sanvito, F., Pala, M., Benedicenti, F., Gallina, P., Sergi, L. S., Merella, S., Bulfone, A., et al. (2013). Lentiviral vector-based insertional mutagenesis identifies genes associated with liver cancer. Nat Methods 10, 155-161. doi.org/10.1038/nmeth.2331.
66. Rasmussen, A. M., Borelli, G., Hoel, H. J., Lislerud, K., Gaudernack, G., Kvalheim, G., and Aarvak, T. (2010). Ex vivo expansion protocol for human tumor specific T cells for adoptive T cell therapy. J Immunol Methods 355, 52-60. doi.org/10.1016/j.jim.2010.02.004.
67. Reynisson, B., Alvarez, B., Paul, S., Peters, B., and Nielsen, M. (2020). NetMHCpan-4.1 and NetMHCIIpan-4.0: improved predictions of MHC antigen presentation by concurrent motif deconvolution and integration of MS MHC eluted ligand data. Nucleic Acids Res 48, W449-W454. doi.org/10.1093/nar/gkaa379.
68. Robbins, P. F., Li, Y. F., El-Gamil, M., Zhao, Y., Wargo, J. A., Zheng, Z., Xu, H., Morgan, R. A., Feldman, S. A., Johnson, L. A., et al. (2008). Single and dual amino acid substitutions in TCR CDRs can enhance antigen-specific T cell functions. J Immunol 180, 6116-6131. doi.org/10.4049/jimmunol.180.9.6116.
69. Robins, H. S., Campregher, P. V., Srivastava, S. K., Wacher, A., Turtle, C. J., Kahsai, O., Riddell, S. R., Warren, E. H., and Carlson, C. S. (2009). Comprehensive assessment of T-cell receptor beta-chain diversity in alphabeta T cells. Blood 114, 4099-4107. doi.org/10.1182/blood-2009-04-217604.
70. Roth et al., Reprogramming human T cell function and specificity with non-viral genome targeting. Nature. 2018 July; 559(7714): 405-409.
71. Rubin, A. J., Parker, K. R., Satpathy, A. T., Qi, Y., Wu, B., Ong, A. J., Mumbach, M. R., Ji, A. L., Kim, D. S., Cho, S. W., et al. (2019). Coupled Single-Cell CRISPR Screening and Epigenomic Profiling Reveals Causal Gene Regulatory Networks. Cell 176, 361-376.e17. doi.org/10.1016/j.cell.2018.11.022.
72. Satpathy, A. T., Saligrama, N., Buenrostro, J. D., Wei, Y., Wu, B., Rubin, A. J., Granja, J. M., Lareau, C. A., Li, R., Qi, Y., et al. (2018). Transcript-indexed ATAC-seq for precision immune profiling. Nat Med 24, 580-590. doi.org/10.1038/s41591-018-0008-8.
73. Solache, A., Morgan, C. L., Dodi, A. I., Morte, C., Scott, I., Baboonian, C., Zal, B., Goldman, J., Grundy, J. E., and Madrigal, J. A. (1999). Identification of three HLA-A*0201-restricted cytotoxic T cell epitopes in the cytomegalovirus protein pp65 that are conserved between eight strains of the virus. J Immunol 163, 5512-5518.
74. Srivastava, A. (2016). In vivo tissue-tropism of adeno-associated viral vectors. Curr Opin Virol 21, 75-80. doi.org/10.1016/j.coviro.2016.08.003.
75. Stano, A., Leaman, D. P., Kim, A. S., Zhang, L., Autin, L., Ingale, J., Gift, S. K., Truong, J., Wyatt, R. T., Olson, A. J., et al. (2017). Dense Array of Spikes on HIV-1 Virion Particles. J Virol 91, e00415-17. doi.org/10.1128/JVI.00415-17.
76. Stoeckius, M., Hafemeister, C., Stephenson, W., Houck-Loomis, B., Chattopadhyay, P. K., Swerdlow, H., Satija, R., and Smibert, P. (2017). Simultaneous epitope and transcriptome measurement in single cells. Nat Methods 14, 865-868. doi.org/10.1038/nmeth.4380.
77. Sturm, G., Szabo, T., Fotakis, G., Haider, M., Rieder, D., Trajanoski, Z., and Finotello, F. (2020). Scirpy: a Scanpy extension for analyzing single-cell T-cell receptor-sequencing data. Bioinformatics 36, 4817-4818. doi.org/10.1093/bioinformatics/btaa611.
78. Su, F. Y., Zhao, Q. H., Dahotre, S. N., Gamboa, L., Bawage, S. S., Silva Trenkle, A. D., Zamat, A., Phuengkham, H., Ahmed, R., Santangelo, P. J., et al. (2022). In vivo mRNA delivery to virus-specific T cells by light-induced ligand exchange of MHC class I antigen-presenting nanoparticles. Sci Adv 8, eabm7950. doi.org/10.1126/sciadv.abm7950.
79. Tan, E. M. (1989). Antinuclear antibodies: diagnostic markers for autoimmune diseases and probes for cell biology. Adv Immunol 44, 93-151. doi.org/10.1016/s0065-2776(08)60641-0.
80. Vance, T. D. R and Lee, J. E. (2020) Virus and eukaryote fusogen superfamilies. Curr. Biol. July 6; 30(13): R750-R754.
81. Verma, I. M., and Somia, N. (1997). Gene therapy—promises, problems and prospects.

Nature 389, 239-242. doi.org/10.1038/38410.
82. Vieyra-Lobato, M. R., Vela-Ojeda, J., Montiel-Cervantes, L., López-Santiago, R., and Moreno-Lafont, M. C. (2018). Description of CD8+ Regulatory T Lymphocytes and Their Specific Intervention in Graft-versus-Host and Infectious Diseases, Autoimmunity, and Cancer. J Immunol Res 2018, 3758713. doi.org/10.1155/2018/3758713.
83. Vita, R., Mahajan, S., Overton, J. A., Dhanda, S. K., Martini, S., Cantrell, J. R., Wheeler, D. K., Sette, A., and Peters, B. (2019). The Immune Epitope Database (IEDB): 2018 update. Nucleic Acids Res 47, D339-D343. doi.org/10.1093/nar/gky1006.
84. Waldman, A. D., Fritz, J. M., and Lenardo, M. J. (2020). A guide to cancer immunotherapy: from T cell basic science to clinical practice. Nat Rev Immunol 20, 651-668. doi.org/10.1038/s41577-020-0306-5.
85. Wang, J. W., Jagu, S., Wu, W. H., Viscidi, R. P., Macgregor-Das, A., Fogel, J. M., Kwak, K., Daayana, S., Kitchener, H., Stern, P. L., et al. (2015). Seroepidemiology of Human Papillomavirus 16 (HPV16) L2 and Generation of L2-Specific Human Chimeric Monoclonal Antibodies. Clin Vaccine Immunol 22, 806-816. doi.org/10.1128/CVI.00799-14.
86. Wildt, S., and Gerngross, T. U. (2005). The humanization of N-glycosylation pathways in yeast. Nat Rev Microbiol 3, 119-128. doi.org/10.1038/nrmicrol087.
87. Wills, M. R., Carmichael, A. J., Mynard, K., Jin, X., Weekes, M. P., Plachter, B., and Sissons, J. G. (1996). The human cytotoxic T-lymphocyte (CTL) response to cytomegalovirus is dominated by structural protein pp65: frequency, specificity, and T-cell receptor usage of pp65-specific CTL. J Virol 70, 7569-7579. doi.org/10.1128/JVI.70.11.7569-7579.1996.
88. Wing, K., Onishi, Y., Prieto-Martin, P., Yamaguchi, T., Miyara, M., Fehervari, Z., Nomura, T., and Sakaguchi, S. (2008). CTLA-4 control over Foxp3+ regulatory T cell function. Science 322, 271-275. doi.org/10.1126/science.1160062.
89. Wolf, F. A., Angerer, P., and Theis, F. J. (2018). SCANPY: large-scale single-cell gene expression data analysis. Genome Biol 19, 15. doi.org/10.1186/s13059-017-1382-0.
90. Wolfert, M. A., and Boons, G. J. (2013). Adaptive immune activation: glycosylation does matter. Nat Chem Biol 9, 776-784. doi.org/10.1038/nchembio.1403.
91. Wu, X., Liu, H., Xiao, H., Kim, J., Seshaiah, P., Natsoulis, G., Boeke, J. D., Hahn, B. H., and Kappes, J. C. (1995). Targeting foreign proteins to human immunodeficiency virus particles via fusion with Vpr and Vpx. J Virol 69, 3389-3398. doi.org/10.1128/JVI.69.6.3389-3398.1995.
92. Yang, L., Bailey, L., Baltimore, D., and Wang, P. (2006). Targeting lentiviral vectors to specific cell types in vivo. Proc Natl Acad Sci USA 103, 11479-11484. doi.org/10.1073/pnas.0604993103.
93. Yassai, M. B., Naumov, Y. N., Naumova, E. N., and Gorski, J. (2009). A clonotype nomenclature for T cell receptors. Immunogenetics 61, 493-502. doi.org/10.1007/s00251-009-0383-x.
94. Yonehara, S., Ishii, A., and Yonehara, M. (1989). A cell-killing monoclonal antibody (anti-Fas) to a cell surface antigen co-downregulated with the receptor of tumor necrosis factor. J Exp Med 169, 1747-1756. doi.org/10.1084/jem.169.5.1747.
95. Yost, K. E., Satpathy, A. T., Wells, D. K., Qi, Y., Wang, C., Kageyama, R., McNamara, K. L., Granja, J. M., Sarin, K. Y., Brown, R. A., et al. (2019). Clonal replacement of tumor-specific T cells following PD-1 blockade. Nat Med 25, 1251-1259. doi.org/10.1038/s41591-019-0522-3.
96. Zhang, H., Sun, M., Wang, J., Zeng, B., Cao, X., Han, Y., Tan, S., and Gao, G. F. (2021). Identification of NY-ESO-1₁₅₇₋₁₆₅ Specific Murine T Cell Receptors With Distinct Recognition Pattern for Tumor Immunotherapy. Front Immunol 12, 644520. doi.org/10.3389/fimmu.2021.644520.
97. Zhang, J., Medaer, R., Stinissen, P., Hafler, D., and Raus, J. (1993). MHC-restricted depletion of human myelin basic protein-reactive T cells by T cell vaccination. Science 261, 1451-1454. doi.org/10.1126/science.7690157.
98. Zhang, S. Q., Ma, K. Y., Schonnesen, A. A., Zhang, M., He, C., Sun, E., Williams, C. M., Jia, W., and Jiang, N. (2018). High-throughput determination of the antigen specificities of T cell receptors in single cells. Nat Biotechnol doi.org/10.1038/nbt.4282.

What is claimed is:

1. An engineered lentivirus comprising:
a heterologous ligand displayed on a surface of the lentivirus;
a fusogen comprising a modified viral envelope protein, wherein the fusogen is capable of fusing the lentivirus to a host cell, wherein the host cell comprises an endogenous receptor for the ligand;
a reporter protein operably linked to a lentiviral structural protein; and
a barcoded RNA.

2. The engineered lentivirus of claim 1, wherein the fusogen is or comprises a modified VSV-G viral envelope protein.

3. The engineered lentivirus of any one of claims 1 or 2, wherein the modified VSV-G viral envelope protein comprises one or more amino acid substitutions at any one of the positions H8, K47, Y209, and R354 of the VSV-G polypeptide.

4. The engineered lentivirus of claim 3, wherein the modified VSV-G viral envelope protein comprises a K47Q substitution and a R354A substitution.

5. The engineered lentivirus of any one of claims 1-4, wherein the ligand is or comprises a protein or an epitope.

6. The engineered lentivirus of any one of claims 1-5, wherein the ligand is or comprises a MHC peptide, an antibody, an antigen, a secreted protein, a cell-surface protein, or other form of antigen that is expressed by a cell.

7. The engineered lentivirus of any one of claims 1-6, wherein the antigen is an intracellular antigen.

8. The engineered lentivirus of any one of claims 1-7, wherein the ligand is operably linked to an optimized transmembrane domain.

9. The engineered lentivirus of claim 8, wherein the optimized transmembrane domain is transmembrane domain derived from HLA-DRA, HLA-DRB, HLA-A2, ICAM1, CD43, CD162, CD62L, CD49d, or LFA-1.

10. The engineered lentivirus of any one of claims 1-9, wherein the ligand is operably linked to an optimized transmembrane domain and a signal peptide.

11. The engineered lentivirus of any one of claims 1-10, comprising a defective integrase protein.

12. The engineered lentivirus of any one of claims 1-11, wherein the reporter protein is GFP or mNeon.

13. The engineered lentivirus of any one of claims 1-12, wherein the structural protein is a nucleocapsid protein.

14. The engineered lentivirus of any one of claims 1-13, wherein the structural protein is a Gag protein.

15. The engineered lentivirus of any one of claims 1-14, wherein the barcoded RNA is encapsulated in viral particles.

16. The engineered lentivirus of any one of claims 1-14, wherein the RNA encodes the ligand protein.

17. The engineered lentivirus of any one of claims 1-14, wherein the RNA encodes a gene of interest to be delivered into host cells.

18. The engineered lentivirus of any one of claims 1-14, wherein the RNA is read out by Next-generation sequencing technology.

19. The engineered lentivirus of any one of claims 1-14, wherein the RNA comprises a capture sequence.

20. A method for identifying a ligand-receptor pair, the method comprising:
providing at least one engineered lentivirus of any one of claims 1-19;
combining the lentivirus with a population of cells; and
sorting the population of cells based on the presence of the reporter gene, thereby identifying a ligand-receptor pair.

21. The method of claim 20, wherein the method comprises providing a pool of engineered lentiviruses, the pool displaying different ligands.

22. The method of claim 20, wherein the method comprises combining the lentivirus with cells and incubating the virus/cell mixture at about 4° C.

23. The method of claim 20, wherein the method comprises combining the lentivirus with cells, and incubating the virus/cell mixture at room temperature.

24. The method of claim 20, wherein the method comprises combining the lentivirus with cells and incubating the virus/cell mixture at about 37° C.

25. The method of claim 20-24, wherein the method comprises incubating the virus/cell mixture for about 30 minutes, or about 1 hour or about 2 hours or any period from about 0.5 hours to about 2.5 hours.

26. The method of any one of claims 20-25, further comprising a step of single-cell sequencing of the viral RNA to identify the ligand sequence.

27. The method of any one of claims 20-26, further comprising a step of single-cell sequencing of the cell's transcriptome to identify the receptor sequence.

28. A method of delivering a nucleic acid of interest or a protein of interest to a user-defined target cell, comprising:
providing the engineered lentivirus of any one of claims 1-19;
contacting the lentivirus with a cell mixture comprising the target cell, and
delivering the nucleic acid or protein only to the target cell, wherein the target cell expresses a receptor specific to the ligand on the lentivirus surface.

29. The method of claim 28, wherein the ligand is modified in order to deliver cargo to the user-defined target cell.

30. The method of claim 29, wherein the nucleic acid of interest is packaged inside the engineered lentiviral particle.

31. The method of claim 29, wherein the protein of interest is operably linked to a lentiviral structural protein of the lentivirus.

32. The method of claim 31, wherein the protein of interest operably linked to a gag protein.

33. The method of any one of claims 28 to 32, wherein the target cell is in vivo, ex vivo, or in vitro.

34. The method of claim any one of claims 28 to 33, wherein the target cell is a mammalian cell.

35. The method of claim 34, wherein the mammalian cell is a human cell.

36. The method of any one of claims 34 to 35, wherein the target cell is an immune cell.

37. The method of claim 36, wherein the immune cell is a T cell or a B cell.

38. The method of claim 35, wherein the human cell is a primary human blood cell (PBMC).

39. A method of identifying an immunogenic antigen, the method comprising:
providing an engineered lentivirus comprising a heterologous receptor protein displayed on the surface of the lentivirus, a fusogen comprising a modified VSV-G viral envelope protein wherein the fusogen is capable of fusing the lentivirus to a host cell, wherein the host cell comprises a native antigen for the receptor, a reporter transgene operably linked to a lentivirus structural protein, and a barcoded RNA, wherein the RNA encodes antigen information;
combining the lentivirus with the population of cells; and
sorting the population of cells based on the presence of the reporter.

40. The method of claim 39, wherein the method further comprises a step for sequencing of the viral RNA to identify the antigen sequence.

41. The method of claim 39, wherein the method further comprises a step for sequencing of the cell's receptor RNA.

42. A method of identifying a T-cell receptor and paired pMHC, the method comprising:
providing an engineered lentivirus comprising a pMHC displayed on virus surface, a fusogen comprising a modified VSV-G viral envelope protein wherein the fusogen is capable of fusing the lentivirus to a host cell, wherein the host cell comprises a T-cell receptor for the PMHC, a reporter transgene operably linked to a lentivirus structural protein, and a barcoded RNA;
combining the lentivirus with the population of cells; and
sorting the population of cells based on the presence of the reporter thereby identifying the T-cell receptor.

43. The method of claim 42, wherein the population of cells comprises human primary T-cells.

44. The method of claim 42, wherein the pMHC is encoded by a RNA comprising a signal peptide, the PMHC, a G4S linker, b2m gene, a G4S linker and a MH allele in tandem.

45. The method of claim 42, wherein the method further comprises a step for single cell sequencing of the viral RNA to identify the MHC peptide sequence.

46. The method of any one of claims 42-45, wherein the method further comprises a step for sequencing of the cell's receptor sequence to identify the MHC peptide and T-cell receptor sequence.

47. A method of identifying a B-cell receptor or antibody, the method comprising:
providing an engineered lentivirus comprising an epitope displayed on lentivirus surface wherein the epitope is operably linked with an ICAM1 transmembrane domain, a fusogen comprising a modified VSV-G viral envelope protein wherein the fusogen is capable of fusing the lentivirus to a host cell, wherein the host cell comprises a B-cell receptor for the intracellular epitope, a reporter transgene operably linked to a lentivirus structural protein, and a barcoded RNA;
combining the lentivirus with the population of B cells; and
sorting the population of cells based on the presence of the reporter thereby identifying the B-cell receptor or antibody.

48. The method of claim 47, wherein the antigen is a cell surface membrane protein, an intracellular protein, a secreted protein, or glycosylated protein.

49. The method of claims 47-48, wherein the method further comprises the step of single cell sequencing of the viral RNA to identify the antigen and matching B-cell receptor sequences.

50. A method of identifying an antigen for a B-cell receptor, the method comprising:
providing the engineered lentivirus of any one of claims 1-19;
combining the lentivirus with the population of B cells; and
sorting the population of cells based on the presence of the reporter thereby identifying the B-cell antigen.

51. A method of single-cell multiomics, comprising:
providing an engineered lentivirus comprising a heterologous ligand displayed on the surface of the lentivirus, a fusogen comprising a modified VSV-G viral envelope protein wherein the fusogen is capable of fusing the lentivirus to a host cell, wherein the host cell comprises an endogenous receptor for the ligand, a reporter transgene operably linked to a lentivirus structural protein, and a RNA, wherein the RNA comprises an antigen sequence and a capture tag for single cell sequencing; and
retrieving transcriptome and phenotype information simultaneously at the single cell level.

52. The method of claim 51, wherein the single cells sequencing is a droplet based platform.

53. The method of claim 51, wherein the cell's phenotype comprise surface markers by CITE-seq.

54. The method of claim 51, wherein the information comprises ligand and receptor sequences.

55. The method of claim 51, wherein the single cells multiomics use whole cell as input.

56. The method of claim 51, wherein the single cells multiomics use whole cell as input and comprises a step of reverse transcription.

57. A method for selectively depleting or enriching a target cell population in a cell mixture, the method comprising:
providing (a) an engineered lentivirus according to any one of claims 1-19, and (b) a cell mixture comprising
(i) a target cell population expressing a receptor specific for the ligand displayed on the surface of the engineered lentivirus, and
(ii) a non-target cell population that does not express the receptor;
contacting the engineered lentivirus with the cell population, and delivering the nucleic acid or protein only to the target cell;
adding a reagent that specifically inhibits growth of the target cell population or inhibits growth of the non-target cell population, thereby selectively depleting or enriching the target cell population.

58. The method of claim 57, the target cell expresses a herpes simplex virus thymidine kinase (HSV-TK) transgene, and the added reagent comprises or is ganciclovir (GCV).

59. The method of any one of claims 57 to 58, wherein the target cell expresses shRNA to decrease expression of cell death receptor FAS to prevent cell death of the target population.

60. The method of any one of claims 57 to 59, wherein the target cell population comprises immune cells.

61. The method of claim 60, wherein the immune cells comprise a T cell.

62. The method of claim 60, wherein the immune cells comprise a B cell.

63. The method of any one of claims 60 to 62, wherein the immune cells are autoreactive immune cells.

64. The method of any one of claims 60 to 63, wherein the immune cells are specific for an antigen associated with a health condition.

65. The method of claim 64, wherein the health condition is a proliferative disorder, inflammatory disorder, autoimmune disorder, or a microbial infection.

66. The method of claim 65, wherein the proliferative disorder is a cancer.

67. The method of claim 65, wherein the microbial infection is a bacterial infection, viral infection, or microfungal infection.


SEQUENCE LISTING (56 sequences)

SEQ ID NO: 1 - protein, 9 aa, synthetic construct
SLLMWITQC

SEQ ID NO: 2 - protein, 9 aa, synthetic construct
SLAMWITQC

SEQ ID NO: 3 - protein, 9 aa, synthetic construct
SLLMWIAQC

SEQ ID NO: 4 - protein, 9 aa, synthetic construct
NLVPMVATV

SEQ ID NO: 5 - protein, 11 aa, synthetic construct
YSEHPTFTSQY

SEQ ID NO: 6 - protein, 9 aa, synthetic construct
GILGFVFTL

SEQ ID NO: 7 - protein, 9 aa, synthetic construct
VLEETSVML

SEQ ID NO: 8 - protein, 9 aa, synthetic construct
VLAELVKQI

SEQ ID NO: 9 - protein, 9 aa, synthetic construct
VLGPISGHV

SEQ ID NO: 10 - protein, 9 aa, synthetic construct
MLNIPSINV

SEQ ID NO: 11 - protein, 9 aa, synthetic construct
FPTKDVALR

SEQ ID NO: 12 - protein, 9 aa, synthetic construct
QIFLEVQAI

SEQ ID NO: 13 - protein, 9 aa, synthetic construct
TPRVTGGGA

SEQ ID NO: 14 - protein, 9 aa, synthetic construct
CLLESVYTA

SEQ ID NO: 15 - protein, 9 aa, synthetic construct
TLIVNLVEV

SEQ ID NO: 16 - protein, 9 aa, synthetic construct
GVLDAVWRV

SEQ ID NO: 17 - protein, 10 aa, synthetic construct
ALWDVALLEV

SEQ ID NO: 18 - protein, 9 aa, synthetic construct
MLNIPSINV

SEQ ID NO: 19 - DNA, 16 bp, synthetic construct
  source 1..16: mol_type = other DNA
gttgaaggcgtttgca

SEQ ID NO: 20 - DNA, 21 bp, synthetic construct
  source 1..21: mol_type = other DNA
tcttctcaacgagtttaacgt

SEQ ID NO: 21 - DNA, 20 bp, synthetic construct
  source 1..20: mol_type = other DNA
ccaccatggcgacgggttca

SEQ ID NO: 22 - DNA, 33 bp, synthetic construct
  source 1..33: mol_type = other DNA
acactctttccctacacgacgctcttccgatct

SEQ ID NO: 23 - DNA, 54 bp, synthetic construct
  source 1..54: mol_type = other DNA
gtgactggagttcagacgtgtgctcttccgatctggttacaggagggctcggca

SEQ ID NO: 24 - DNA, 25 bp, synthetic construct
  source 1..25: mol_type = other DNA
acactctttccctacacgacgctct

SEQ ID NO: 25 - DNA, 22 bp, synthetic construct
  source 1..22: mol_type = other DNA
aacgtcacaggagctttcggga

SEQ ID NO: 26 - DNA, 20 bp, synthetic construct
  source 1..20: mol_type = other DNA
tgaaggcgtttgcacatgca

SEQ ID NO: 27 - DNA, 57 bp, synthetic construct
  source 1..57: mol_type = other DNA
gtgactggagttcagacgtgtgctcttccgatctgggttttggatgtacggatgaac

SEQ ID NO: 28 - DNA, 52 bp, synthetic construct
  source 1..52: mol_type = other DNA
gtgactggagttcagacgtgtgctcttccgatctcagctggtacacggcagg

SEQ ID NO: 29 - DNA, 15 bp, synthetic construct
  source 1..15: mol_type = other DNA
tcccatataagaaac

SEQ ID NO: 30 - DNA, 25 bp, synthetic construct
  source 1..25: mol_type = other DNA
aagcagtggtatcaacgcagagtac

SEQ ID NO: 31 - DNA, 48 bp, synthetic construct
  source 1..48: mol_type = other DNA
cccttgtgtttggaattataactcgagttataattccaaacacaaggg

SEQ ID NO: 32 - DNA, 48 bp, synthetic construct
  source 1..48: mol_type = other DNA
gcgtatgacacattgattaaactcgagtttaatcaatgtgtcatacgc

SEQ ID NO: 33 - DNA, 48 bp, synthetic construct
  source 1..48: mol_type = other DNA
cctgaaacagtggcaataaatctcgagatttattgccactgtttcagg

SEQ ID NO: 34 - protein, 21 aa, synthetic construct
LLAILILALVATIFFVCTVVL

SEQ ID NO: 35 - protein, 21 aa, synthetic construct
YLYVLSGIGGLLLLLLIFIVL

SEQ ID NO: 36 - protein, 23 aa, synthetic construct
PLFIPVAVMVTAFSGLAFIIWLA

SEQ ID NO: 37 - protein, 24 aa, synthetic construct
IVIISSSLLLGLIVLLLISYVMWK

SEQ ID NO: 38 - protein, 23 aa, synthetic construct
GMLPVAVLVALLAVIVLVALLLL

SEQ ID NO: 39 - protein, 25 aa, synthetic construct
IVGIIAGLVLFGAVITGAVVAAVMW

SEQ ID NO: 40 - protein, 23 aa, synthetic construct
FFFIIGLIIGLFLVLRVGIHLCI

SEQ ID NO: 41 - protein, 23 aa, synthetic construct
IVIITVVAAAVIMGTAGLSTYLY

SEQ ID NO: 42 - protein, 23 aa, synthetic construct
NVVCALGLTVGLVGIIIGTIFII

SEQ ID NO: 43 - protein, 26 aa, synthetic construct
GTVVVISAILALVVLTIISLIILIML

SEQ ID NO: 44 - protein, 15 aa, synthetic construct
AVRLSRKGHMYPVRN

SEQ ID NO: 45 - protein, 11 aa, synthetic construct
YKVGFFKRNLK

SEQ ID NO: 46 - protein, 11 aa, synthetic construct
RRLKKGKKSKR

SEQ ID NO: 47 - protein, 10 aa, synthetic construct
AGFFKRQYKS

SEQ ID NO: 48 - protein, 9 aa, synthetic construct
WRRRQKRRT

SEQ ID NO: 49 - protein, 9 aa, synthetic construct
RRKSSDRKG

SEQ ID NO: 50 - protein, 10 aa, synthetic construct
KLKHTKKRQI

SEQ ID NO: 51 - protein, 11 aa, synthetic construct
NRQRKIKKYRL

SEQ ID NO: 52 - protein, 15 aa, synthetic construct
KGLRKSNAAERRGPL

SEQ ID NO: 53 - protein, 6 aa, synthetic construct
WQKKPR

SEQ ID NO: 54 - protein, 13 aa, synthetic construct
CASSFQGYTEAFF

SEQ ID NO: 55 - protein, 15 aa, synthetic construct
CASSYQTGASYGYTF

SEQ ID NO: 56 - protein, 511 aa, Homo sapiens
  REGION 1..511: note = MISC_FEATURE - vesicular stomatitis virus G protein (VSV-G)
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK